(19) United States
(12) Patent Application Publication
(10) Pub. No.: US 2015/0297741 A1
(43) Pub. Date: Oct. 22, 2015

(54) BIO-ORTHOGONAL DRUG ACTIVATION

(71) Applicant: TAGWORKS PHARMACEUTICALS B.V., Eindhoven (NL)

(72) Inventor: Marc Stefan Robillard, Eindhoven (NL)

(73) Assignee: Tagworks Pharmaceuticals B.V., Eindhoven (NL)

(21) Appl. No.: 14/646,966

(22) PCT Filed: Nov. 22, 2013
(86) PCT No.: PCT/NL2013/050848

§ 371 Date: May 22, 2015

(30) Foreign Application Priority Data
Nov. 22, 2012 (EP) 12193918.5

(51) Int. Cl. A61K 47/48; C07K 16/30; A61K 31/704

(52) U.S. Cl. CPC A61K 47/48246; A61K 31/704; A61K 47/48061; C07K 16/30; C07K 2317/626; C07K 2317/76

(57) ABSTRACT

Disclosed is a kit for the administration and activation of a Prodrug. The kit comprises a Masking Moiety linked, directly or indirectly, to a Trigger moiety, which in turn is linked to a Drug, and an Activator for the Trigger moiety. The Trigger moiety comprises a dienophile and the Activator comprises a diene, whereby the dienophile is an eight-membered non-aromatic cyclic alkenylene group, preferably a cyclooctene group, and more preferably a trans-cyclooctene group. The Trigger and the Activator undergo a fast, bio-orthogonal reaction resulting in the release of the Masking Moiety, and activation of the drug.

### CROSS-REFERENCES TO RELATED APPLICATIONS

This application claims priority to and hereby incorporates by reference in their entirety PCT application number PCT/NL2013/050848 entitled “BIO-ORTHOGONAL DRUG ACTIVATION” filed on Nov. 22, 2013; and European Patent Application EP 12193918.5 entitled “BIO-ORTHOGONAL DRUG ACTIVATION” filed on Nov. 22, 2012.

### TECHNICAL FIELD

The invention relates to therapeutical methods on the basis of inactivated drugs, such as prodrugs, that are activated by means of an abiotic, bio-orthogonal chemical reaction.

### BACKGROUND OF THE INVENTION

In the medical arena the use of inactive compounds such as prodrugs which are activated in a specific site in the human or animal body is well known. Also targeted delivery of inactives such as prodrugs has been studied extensively. Much effort has been devoted to drug delivery systems that effect drug release selectivity at a target site and/or at a desired moment in time. One way is to selectively activate a (systemic) prodrug specifically by local and specific enzymatic activity. However, in many cases a target site of interest lacks a suitable overexpressed enzyme. An alternative is to transport an enzyme to target tissue via a technique called antibody-directed enzyme prodrug therapy (ADEPT). In this approach an enzyme is targeted to a tumor site by conjugation to an antibody that binds a tumor-associated antigen. After systemic administration of the conjugate, its localization at the target and clearance of unbound conjugate, a designed prodrug is administered systemically and locally activated. This method requires the catalysis of a reaction that must not be accomplished by an endogenous enzyme. Enzymes of non-mammalian origin that meet these needs are likely to be highly immunogenic, a fact that makes repeated administration impossible. Alternatively, prodrugs can be targeted to a disease site followed by disease-specific or -nonspecific endogenous activation processes (eg pH, enzymes, thiol-containing compounds).

Targeted anticancer therapeutics are designed to reduce nonspecific toxicities and increase efficacy relative to conventional cancer chemotherapy. This approach is embodied by the powerful targeting ability of monoclonal antibodies (mAbs) to specifically deliver highly potent, conjugated small molecule therapeutics to a cancer cell. In an attempt to address the issue of toxicity, chemotherapeutic agents (drugs) have been coupled to targeting molecules such as antibodies or protein receptor ligands that bind with a high degree of specificity to tumor cell to form compounds referred to as antibody-drug conjugates (ADC) or immunoconjugates. Immunoconjugates in theory should be less toxic because they direct the cytotoxic drug to tumors that express the particular cell surface antigen or receptor. This strategy has met limited success in part because cytotoxic drugs tend to be inactive or less active when conjugated to large antibodies, or protein receptor ligands. Promising advancements with immunoconjugates has seen cytotoxic drugs linked to antibodies through a linker that is cleaved at the tumor site or inside tumor cells (Senter et al, Current Opinion in Chemical Biology 2010, 14:529-537). Ideally, the mAb will specifically bind to an antigen with substantial expression on tumor cells but limited expression on normal tissues. Specificity allows the utilization of drugs that otherwise would be too toxic for clinical application. Most of the recent work in this field has centered on the use of highly potent cytotoxic agents. This requires the development of linker technologies that provide conditional stability, so that drug release occurs after tumor binding, rather than in circulation.

As a conjugate the drug is inactive but upon target localization the drug is released by eg pH or an enzyme, which could be target specific but may also be more generic. The drug release may be achieved by an extracellular mechanism such as low pH in tumor tissue, hypoxia, certain enzymes, but in general more selective drug release can be achieved through intracellular, mostly lysosomal, release mechanisms (e.g. glutathione, proteases, catabolism) requiring the antibody conjugate to be first internalized. Specific intracellular release mechanisms (eg glutathione, cathepsin) usually result in the parent drug, which depending on its properties, can escape the cell and attack neighboring cells. This is viewed as an important mechanism of action for a range of antibody-drug conjugates, especially in tumors with heterogeneous receptor expression, or with poor mAb penetration. Examples of cleavable linkers are: hydrazones (acid labile), peptide linkers (cathepsin B cleavable), hindered disulfide moieties (thiol cleavable). Also non-cleavable linkers can be used in mAb-drug conjugates. These constructs release their drug upon catabolism, presumably resulting in a drug molecule still attached to one amino acid. Only a subset of drugs will regain their activity as such a conjugate. Also, these aminoacid-linked drugs cannot escape the cells. Nevertheless, as the linker is stable, these constructs are generally regarded as the safest and depending on the drug and target, can be very effective.

The current antibody-drug conjugate release strategies have their limitations. The extracellular drug release mechanisms are usually too unspecific (as with pH sensitive linkers) resulting in toxicity. Intracellular release depends on efficient (e.g receptor-mediated internalization) of the mAb-drug, while several cancers lack cancer-specific and efficiently internalizing targets that are present in sufficiently high copy numbers. Intracellular release may further depend on the presence of an activating enzyme (proteases) or molecules (thiols such as glutathione) in sufficiently high amount. Following intracellular release, the drug may, in certain cases, escape from the cell to target neighbouring cells. This effect is deemed advantageous in heterogeneous tumors where not every cell expresses sufficiently high amounts of target receptor. It is of further importance in tumors that are difficult to penetrate due e.g. to elevated interstitial pressure, which impedes convectional flow. This is especially a problem for large constructs like mAb (conjugates). This mechanism is also essential in cases where a binding site barrier occurs. Once a targeted agent leaves the vasculature and binds to a receptor, its movement within the tumor will be restricted. The likelihood of a mAb conjugate being restricted in the perivascular space scales with its affinity for its target. The penetration can be improved by increasing the mAb dose, however, this approach is limited by dose limiting toxicity in e.g. the liver. Further, antigens that are shed from dying cells can be present in the tumor interstitial space where they can prevent mAb-conjugates of binding their target cell. Also, many targets are hampered by ineffective internalization, and different drugs cannot be linked to a mAb in the same way. Further, it has been proven cumbersome to design linkers to be selectively cleavable by endogenous elements in the target while stable to endogenous elements en route to the target (especially the case for slow clearing full mAbs). As a result, the optimal drug, linker, mAb, and target combination needs to be selected and optimized on a case by case basis.

Another application area that could benefit from an effective prodrug approach is the field of T-cell engaging antibody constructs (e.g., bi- or trispecific antibody fragments), which act on cancer by engaging the immunesystem. It has long been considered that bringing activated T-cells into direct contact with cancer cells offers a potent way of killing them (Thompson et al., Biochemical and Biophysical Research Communications 366 (2008) 526-531). Of the many bispecific antibodies that have been created to do this, the majority are composed of two antibody binding sites, one site targets the tumor and the other targets a T-cell (Thakur et al. Current Opinion in Molecular Therapeutics 2010, 12(3), 340-349). However, with bispecific antibodies containing an active T-cell binding site, peripheral T-cell binding will occur. This not only prevents the conjugate from getting to the tumor but can also lead to cytokine storms and T-cell depletion. Photo-activatable anti-T-cell antibodies, in which the anti-T-cell activity is only restored when and where it is required (i.e. after tumor localization via the tumor binding arm), following irradiation with UV light, has been used to overcome these problems. Anti-human CD3 (T-cell targeting) antibodies could be reversibly inhibited with a photocleavable 1-(2-nitrophenyl)ethanol (NPE) coating (Thompson et al., Biochemical and Biophysical Research Communications 366 (2008) 526-531). However, light based activation is limited to regions in the body where light can penetrate, and is not easily amendable to treating systemic disease such as metastatic cancer. Strongly related constructs that could benefit from a prodrug approach are trispecific T-cell engaging antibody constructs with for example a CD3- and a CD28 T-cell engaging moiety in addition to a cancer targeting agent. Such constructs are too toxic to use as such and either the CD3 or the CD28 or both binding domains need to be masked.

Hydrophilic polymers, such as polyethylene glycol (PEG), have been used as a masking moiety of various substrates, such as polypeptides, drugs and liposomes, in order to reduce immunogenicity of the substrate and/or to improve its blood circulation lifetime. For example, parenterally administered proteins can be immunogenic and may have a short pharmacological half-life. Proteins can also be relatively water insoluble. Consequently, it can be difficult to achieve therapeutically useful blood levels of the proteins in patients. Conjugation of PEG to proteins has been described as an approach to overcoming these difficulties. Davis et al. in U.S. Pat. No. 4,179,337 disclose conjugating PEG to proteins such as enzymes and insulin to form PEG-protein conjugates having less immunogenicity yet which retain a substantial proportion of physiological activity. Veronese et al. (Applied Biochem. and Biotech, 11:141-152 (1985)) disclose activating polyethylene glycols with phenyl chloroformates to modify a ribonuclease and a superoxide dimutase. Katre et al. in U.S. Pat. Nos. 4,766,106 and 4,917,888 disclose solubilizing proteins by polymer conjugation. PEG and other polymers are conjugated to recombinant proteins to reduce immunogenicity and increase half-life. (Nitecki et al., U.S. Pat. No. 4,902,502; Enzon, Inc., PCT/US90/02133). Garman (U.S. Pat. No. 4,935,465) describes proteins modified with a water soluble polymer joined to the protein through a reversible linking group. Cleavable PEG masking moieties have been applied, using the same (pH, thiol, enzyme) strategies as described for th ADC field, with the same drawbacks. Effective in vivo PEG cleavage strategies for protein constructs would allow spatial and temporal control over protein activity, toxicity, immunogenicity and pharmacokinetics.

It is desirable to be able to activate targeted drugs selectively and predictably at the target site without being dependent on homogenous penetration and targeting, and on endogenous parameters which may vary en route to and within the target, and from indication to indication and from patient to patient.

In order to avoid the drawbacks of current prodrug activation, it has been proposed in Bioconjugate Chem 2008, 19, 714-718, to make use of an abiotic, bio-orthogonal chemical reaction, viz. the Staudinger reaction, to provoke activation of the prodrug. Briefly, in the introduced concept, the Prodrug is a conjugate of a Drug and a Trigger, and this Drug-Trigger conjugate is not activated endogeneously by e.g. an enzyme or a specific pH, but by a controlled administration of the Activator, i.e. a species that reacts with the Trigger moiety in the Prodrug, to induce release of the Drug from the Trigger (or vice versa, release of the Trigger from the Drug, however one may view this release process). The presented Staudinger approach for this concept, however, has turned out not to work well, and its area of applicability is limited in view of the specific nature of the release mechanism imposed by the Staudinger reaction. Other drawbacks for use of Staudinger reactions are their limited reaction rates, and the oxidative instability of the phosphine components of these reactions. Therefore, it is desired to provide reactants for an abiotic, bio-orthogonal reaction that are stable in physiological conditions, that are more reactive towards each other, and that are capable of inducing release of a bound drug by means of a variety of mechanisms, thus offering a greatly versatile activated drug release method.

The use of a biocompatible chemical reaction that does not rely on endogenous activation mechanisms (eg pH, enzymes) for selective Prodrug activation would represent a powerful new tool in cancer therapy. Selective activation of Prodrugs when and where required allows control over many processes within the body, including cancer. Therapies, such as anti-tumor antibody therapy, may thus be made more specific, providing an increased therapeutic contrast between normal cells and tumour to reduce unwanted side effects. In the context of T-cell engaging anticancer antibodies, the present invention allows the systemic administration and tumor targeting of an masked inactive antibody construct (i.e. this is then the Prodrug), diminishing off-target toxicity. Upon sufficient tumor uptake and clearance from non target areas, the tumor-bound antibody is activated by administration of the Activator, which reacts with the Trigger or Triggers on the antibody or particular antibody domain, resulting in removal of the Masking Moiety and restoration of the T-cell binding function. This results in T-cell activation and anticancer action.

### BRIEF SUMMARY OF THE INVENTION

In order to better address one or more of the foregoing desires, the present invention provides a kit for the administration and activation of a Prodrug, the kit comprising a Masking Moiety linked, directly or indirectly, to a Trigger moiety, which in turn is linked to a Drug, and an Activator for the Trigger moiety, wherein the Trigger moiety comprises a dienophile and the Activator comprises a diene, the dienophile satisfying the following formula (1a):

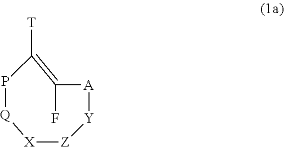

In another aspect, the invention presents a Prodrug comprising a Masking Moiety linked, directly or indirectly, to a trans-cyclooctene moiety satisfying the above formula (1a).

In yet another aspect, the invention provides a method of modifying a Masking Moiety into a Prodrug that can be triggered by an abiotic, bio-orthogonal reaction, the method comprising the steps of providing a Masking Moiety and chemically linking the Masking Moiety to a cyclic moiety satisfying the above formula (1a).

In a still further aspect, the invention provides a method of treatment wherein a patient suffering from a disease that can be modulated by a drug, is treated by administering, to said patient, a Prodrug comprising a Trigger moiety after activation of which by administration of an Activator the Masking Moiety will be released, wherein the Trigger moiety comprises a ring structure satisfying the above formula (1a).

In a still further aspect, the invention is a compound comprising an eight-membered non-aromatic cyclic mono-alkenylene moiety (preferably a cyclooctene moiety, and more preferably a trans-cyclooctene moiety), said moiety comprising a linkage to a Masking Moiety, for use in prodrug therapy in an animal or a human being.

In another aspect, the invention is the use of a diene, preferably a tetrazine as an activator for the release, in a physiological environment, of a substance linked to a compound satisfying formula (1a). In connection herewith, the invention also pertains to a tetrazine for use as an activator for the release, in a physiological environment, of a substance linked to a compound satisfying formula (1a), and to a method for activating, in a physiological environment, the release of a substance linked to a compound satisfying formula (1a), wherein a tetrazine is used as an activator.

In another aspect, the invention presents the use of the inverse electron-demand Diels-Alder reaction between a compound satisfying formula (1a) and a diene, preferably a tetrazine, as a chemical tool for the release, in a physiological environment, of a substance administered in a covalently bound form, wherein the substance is bound to a compound satisfying formula (1a).

### The Retro Diels-Alder Reaction

The dienophile of formula (1a) and the diene are capable of reacting in an inverse electron-demand Diels-Alder reaction. Activation of the Prodrug by the retro Diels-Alder reaction of the Trigger with the Activator leads to release of the Masking Moiety.

Below a reaction scheme is given for a [4+2] Diels-Alder reaction between the (3,6)-di-(2-pyridyl)-s-tetrazine diene and a trans-cyclooctene dienophile, followed by a retro Diels Alder reaction in which the product and dinitrogen is formed. The reaction product may tautomerize, and this is also shown in the scheme. Because the trans-cyclooctene derivative does not contain electron withdrawing groups as in the classical Diels Alder reaction, this type of Diels Alder reaction is distinguished from the classical one, and frequently referred to as an “inverse electron demand Diels Alder reaction”. In the following text the sequence of both reaction steps, i.e. the initial Diels-Alder cyclo-addition (typically an inverse electron demand Diels Alder cyclo-addition) and the subsequent retro Diels Alder reaction will be referred to in shorthand as “retro Diels Alder reaction” or “retro-DA”. It will sometimes be abbreviated as “rDA” reaction. The product of the reaction is then the retro Diels-Alder adduct, or the rDA adduct.

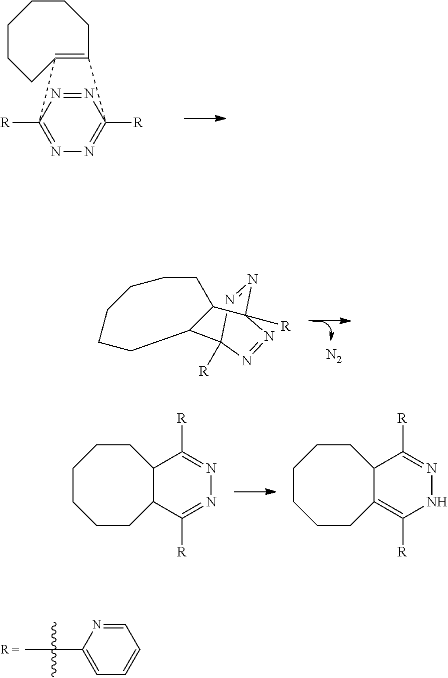

### DETAILED DESCRIPTION OF THE INVENTION

In a general sense, the invention is based on the recognition that a Masking Moiety can be released from trans-cyclooctene derivatives satisfying formula (1a) upon cyclooaddition with compatible dienes, such as tetrazine derivatives. The dienophiles of formula (1a) have the advantage that they react (and effectuate Masking Moiety release) with substantially any diene.

Without wishing to be bound by theory, the inventors believe that the molecular structure of the retro Diels-Alder adduct is such that a spontaneous elimination reaction within this rDA adduct releases the Masking Moiety. Particularly, the inventors believe that appropriately modified rDA components lead to rDA adducts wherein the bond to the Masking Moiety on the dienophile is destabilized by the presence of a lone electron pair on the diene. Alternatively, the inventors believe that the molecular structure of the retro Diels-Alder adduct is such that a spontaneous elimination or cyclization reaction within this rDA adduct releases the Masking Moiety. Particularly, the inventors believe that appropriately modified rDA components, i.e. according to the present invention, lead to rDA adducts wherein the bond to the Masking Moiety on the part originating from the dienophile is broken by the reaction with a nucleophile on the part originating from the dienophile, while such an intramolecular reaction within the part originating from the dienophile is precluded prior to rDA reaction with the diene.

The general concept of using the retro-Diels Alder reaction in Prodrug activation is illustrated in Scheme 1.

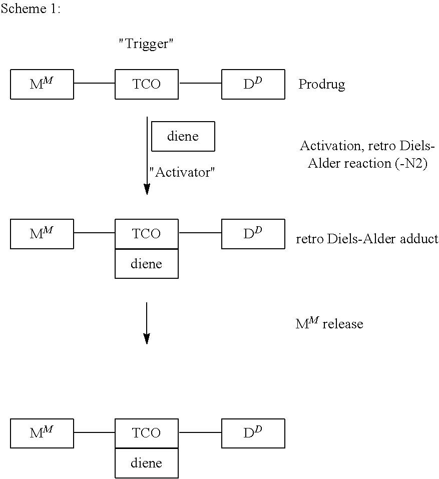

In this scheme “TCO” stands for trans-cyclooctene. The term trans-cyclooctene is used here as possibly including one or more hetero-atoms, and particularly refers to a structure satisfying formula (1a). In a broad sense, the inventors have found that—other than the attempts made on the basis of the Staudinger reaction—the selection of a TCO as the trigger moiety for a prodrug, provides a versatile tool to render drug (active) moieties into prodrug (activatable) moieties, wherein the activation occurs through a powerful, abiotic, bio-orthogonal reaction of the dienophile (Trigger) with the diene (Activator), viz the aforementioned retro Diels-Alder reaction, and wherein the Prodrug is a Drug-dienophile conjugate.

It will be understood that in Scheme 1 in the retro Diels-Alder adduct as well as in the end product, the indicated TCO group and the indicated diene group are the residues of, respectively, the TCO and diene groups after these groups have been converted in the retro Diels-Alder reaction.

A requirement for the successful application of an abiotic bio-orthogonal chemical reaction is that the two participating functional groups have finely tuned reactivity so that interference with coexisting functionality is avoided. Ideally, the reactive partners would be abiotic, reactive under physiological conditions, and reactive only with each other while ignoring their cellular/physiological surroundings (bio-orthogonal). The demands on selectivity imposed by a biological environment preclude the use of most conventional reactions.

The inverse electron demand Diels Alder reaction, however, has proven utility in animals at low concentrations and semi-equimolar conditions (R. Rossin et al, Angewandte Chemie Int Ed 2010, 49, 3375-3378). The reaction partners subject to this invention are strained trans-cyclooctene (TCO) derivatives and suitable dienes, such as tetrazine derivatives. The cycloaddition reaction between a TCO and a tetrazine affords an intermediate, which then rearranges by expulsion of dinitrogen in a retro-Diels-Alder cycloaddition to form a dihydropyridazine conjugate. This and its tautomers is the retro Diels-Alder adduct.

The present inventors have come to the non-obvious insight, that the structure of the TCO of formula (1a), par excellence, is suitable to provoke the release of a Masking Moiety linked to it, as a result of the reaction involving the double bond available in the TCO dienophile, and a diene. The features believed to enable this are (a) the nature of the rDA reaction, which involves a re-arrangement of double bonds, which can be put to use in provoking an elimination cascade; (b) the nature of the rDA adduct that bears a dihydro pyridazine group that is non-aromatic (or another non-aromatic group) and that can rearrange by an elimination reaction to form conjugated double bonds or to form an (e.g. pyridazine) aromatic group, (c) the nature of the rDA adduct that may bear a dihydro pyridazine group that is weakly basic and that may therefore catalyze elimination reactions.

Alternatively, the feature believed to enable this is the change in nature of the eight membered ring of the TCO in the dienophile reactant as compared to that of the eight membered ring in the rDA adduct. The eight membered ring in the rDA adduct has significantly more conformational freedom and has a significantly different conformation as compared to the eight membered ring in the highly strained TCO prior to rDA reaction. A nucleophilic site in the dienophile prior to rDA reaction is locked within the specific conformation of the dienophile and is therefore not properly positioned to react intramolecularly and to thereby release the Masking Moiety. In contrast, and due to the changed nature of the eight membered ring, this nucleophilic site is properly positioned within the rDA adduct and will react intramolecularly, thereby releasing the Masking Moiety. According to the above, but without being limited by theory, we believe that Masking Moiety release is mediated by strain-release of the TCO-dienophile after and due to the rDA reaction with the diene Activator.

In a broad sense, the invention puts to use the recognition that the rDA reaction, using a dienophile of formula (1a), as well as the rDA adduct embody a versatile platform for enabling provoked drug release in a bioorthogonal reaction.

The fact that the reaction is bio-orthogonal, and that many structural options exist for the reaction pairs, will be clear to the skilled person. E.g., the rDA reaction is known in the art of pre-targeted medicine. Reference is made to, e.g., WO 2010/119382, WO 2010/119389, and WO 2010/051530. Whilst the invention presents an entirely different use of the reaction, it will be understood that the various structural possibilities available for the rDA reaction pairs as used in pre-targeting, are also available in the field of the present invention.

The dienophile trigger moiety used in the present invention comprises a trans-cyclooctene ring, the ring optionally including one or more hetero-atoms. Hereinafter this eight-membered ring moiety will be defined as a trans-cyclooctene moiety, for the sake of legibility, or abbreviated as “TCO” moiety. It will be understood that the essence resides in the possibility of the eight-membered ring to act as a dienophile and to be released from its conjugated Masking Moiety upon reaction. The skilled person is familiar with the fact that the dienophile activity is not necessarily dependent on the presence of all carbon atoms in the ring, since also heterocyclic monoalkenylene eight-membered rings are known to possess dienophile activity.

Thus, in general, the invention is not limited to strictly drug-substituted trans-cyclooctene. The person skilled in organic chemistry will be aware that other eight-membered ring-based dienophiles exist, which comprise the same endocyclic double bond as the trans-cyclooctene, but which may have one or more heteroatoms elsewhere in the ring. I.e., the invention generally pertains to eight-membered non-aromatic cyclic alkenylene moieties, preferably a cyclooctene moiety, and more preferably a trans-cyclooctene moiety, comprising a conjugated Masking Moiety.

Other than is the case with e.g. medicinally active substances, where the in vivo action is often changed with minor structural changes, the present invention first and foremost requires the right chemical reactivity combined with an appropriate design of the Masking Moiety—drug-conjugate. Thus, the possible structures extend to those of which the skilled person is familiar with that these are reactive as dienophiles.

It should be noted that, depending on the choice of nomenclature, the TCO dienophile may also be denoted E-cyclooctene. With reference to the conventional nomenclature, it will be understood that, as a result of substitution on the cyclooctene ring, depending on the location and molecular weight of the substituent, the same cyclooctene isomer may formally become denoted as a Z-isomer. In the present invention, any substituted variants of the invention, whether or not formally “E” or “Z,” or “cis” or “trans” isomers, will be considered derivatives of unsubstituted trans-cyclooctene, or unsubstituted E-cyclooctene. The terms “trans-cyclooctene” (TCO) as well as E-cyclooctene are used interchangeably and are maintained for all dienophiles according to the present invention, also in the event that substituents would formally require the opposite nomenclature. I.e., the invention relates to cyclooctene in which carbon atoms 1 and 6 as numbered below are in the E (entgegen) or trans position.

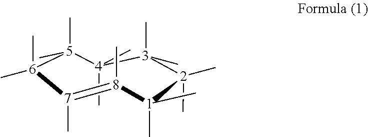

The present invention will further be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where an indefinite or definite article is used when referring to a singular noun e.g. “a” or “an”, “the”, this includes a plural of that noun unless something else is specifically stated.

It is furthermore to be noticed that the term “comprising”, used in the description and in the claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In several chemical formulae below reference is made to “alkyl” and “aryl.” In this respect “alkyl”, each independently, indicates an aliphatic, straight, branched, saturated, unsaturated and/or or cyclic hydrocarbyl group of up to ten carbon atoms, possibly including 1-10 heteroatoms such as O, N, or S, and “aryl”, each independently, indicates an aromatic or heteroaromatic group of up to twenty carbon atoms, that possibly is substituted, and that possibly includes 1-10 heteroatoms such as O, N, P or S. “Aryl” groups also include “alkylaryl” or “arylalkyl” groups (simple example: benzyl groups). The number of carbon atoms that an “alkyl”, “aryl”, “alkylaryl” and “arylalkyl” contains can be indicated by a designation preceding such terms (i.e. C₁₋₁₀ alkyl means that said alkyl may contain from 1 to 10 carbon atoms). Certain compounds of the invention possess chiral centers and/or tautomers, and all enantiomers, diasteriomers and tautomers, as well as mixtures thereof are within the scope of the invention. In several formulae, groups or substituents are indicated with reference to letters such as “A”, “B”, “X”, “Y”, and various (numbered) “R” groups. The definitions of these letters are to be read with reference to each formula, i.e. in different formulae these letters, each independently, can have different meanings unless indicated otherwise.

In all embodiments of the invention as described herein, alkyl is preferably lower alkyl (C₁₋₄ alkyl), and each aryl preferably is phenyl.

Earlier work (R. Rossin et al, Angewandte Chemie Int Ed 2010, 49, 3375-3378) demonstrated the utility of the inverse-electron-demand Diels Alder reaction for pretargeted radioimmunoimaging. This particular cycloaddition example occurred between a (3,6)-di-(2-pyridyl)-s-tetrazine derivative and a E-cyclooctene, followed by a retro Diels Alder reaction in which the product and nitrogen is formed. Because the trans cyclooctene derivative does not contain electron withdrawing groups as in the classical Diels Alder reaction, this type of Diels Alder reaction is distinguished from the classical one, and frequently referred to as an “inverse electron demand Diels Alder reaction”. In the following text the sequence of both reaction steps, i.e. the initial Diels-Alder cyclo-addition (typically an inverse electron demand Diels Alder cyclo-addition) and the subsequent retro Diels Alder reaction will be referred to in shorthand as “retro Diels Alder reaction.”

### Retro Diels-Alder Reaction

The Retro Diels-Alder coupling chemistry generally involves a pair of reactants that couple to form an unstable intermediate, which intermediate eliminates a small molecule (depending on the starting compounds this may be e.g. N₂, CO₂, RCN), as the sole by-product through a retro Diels-Alder reaction to form the retro Diels-Alder adduct. The paired reactants comprise, as one reactant (i.e. one Bio-orthogonal Reactive Group), a suitable diene, such as a derivative of tetrazine, e.g. an electron-deficient tetrazine and, as the other reactant (i.e. the other Bio-orthogonal Reactive Group), a suitable dienophile, such as a strained cyclooctene (TCO).

The exceptionally fast reaction of e.g. electron-deficient (substituted) tetrazines with a TCO moiety results in a ligation intermediate that rearranges to a dihydropyridazine retro Diels-Alder adduct by eliminating N₂ as the sole by-product in a [4+2] Retro Diels-Alder cycloaddition. In aqueous environment, the initially formed 4,5-dihydropyridazine product may tautomerize to a 1,4-dihydropyridazine product.

The two reactive species are abiotic and do not undergo fast metabolism or side reactions in vivo. They are bio-orthogonal, e.g. they selectively react with each other in physiologic media. Thus, the compounds and the method of the invention can be used in a living organism. Moreover, the reactive groups are relatively small and can be introduced in biological samples or living organisms without significantly altering the size of biomolecules therein. References on the Inverse electron demand Diels Alder reaction, and the behavior of the pair of reactive species include: Thalhammer, F; Wallfahrer, U; Sauer, J, Tetrahedron Letters, 1990, 31 (47), 6851-6854; Wijnen, J W; Zavarise, S; Engberts, JBFN, Journal Of Organic Chemistry, 1996, 61, 2001-2005; Blackman, M L; Royzen, M; Fox, J M, Journal Of The American Chemical Society, 2008, 130 (41), 13518-19), R. Rossin, P. Renart Verkerk, Sandra M. van den Bosch, R. C. M. Vulders, I. Verel, J. Lub, M. S. Robillard, Angew Chem Int Ed 2010, 49, 3375, N. K. Devaraj, R. Upadhyay, J. B. Haun, S. A. Hilderbrand, R. Weissleder, Angew Chem Int Ed 2009, 48, 7013, and Devaraj et al., Angew. Chem. Int. Ed., 2009, 48, 1-5.

It will be understood that, in a broad sense, according to the invention the aforementioned retro Diels-Alder coupling and subsequent drug activation chemistry can be applied to basically any pair of molecules, groups, or moieties that are capable of being used in Prodrug therapy. I.e. one of such a pair will comprise a Masking Moiety linked to a dienophile (the Trigger). The other one will be a complementary diene for use in reaction with said dienophile.

### Trigger

The Prodrug comprises a Masking Moiety denoted as M^{M} linked, directly or indirectly, to a Trigger moiety denoted as T^{R}, wherein the Trigger moiety is a dienophile, which is further linked to a Drug D^{D}. The dienophile, in a broad sense, is an eight-membered non-aromatic cyclic alkenylene moiety (preferably a cyclooctene moiety, and more preferably a trans-cyclooctene moiety). Optionally, the trans-cyclooctene (TCO) moiety comprises at least two exocyclic bonds fixed in substantially the same plane, and/or it optionally comprises at least one substituent in the axial position, and not the equatorial position. The person skilled in organic chemistry will understand that the term “fixed in substantially the same plane” refers to bonding theory according to which bonds are normally considered to be fixed in the same plane. Typical examples of such fixations in the same plane include double bonds and strained fused rings. E.g., the at least two exocyclic bonds can be the two bonds of a double bond to an oxygen (i.e. C═O). The at least two exocyclic bonds can also be single bonds on two adjacent carbon atoms, provided that these bonds together are part of a fused ring (i.e. fused to the TCO ring) that assumes a substantially flat structure, therewith fixing said two single bonds in substantially one and the same plane. Examples of the latter include strained rings such as cyclopropyl and cyclobutyl. Without wishing to be bound by theory, the inventors believe that the presence of at least two exocyclic bonds in the same plane will result in an at least partial flattening of the TCO ring, which can lead to higher reactivity in the retro-Diels-Alder reaction.

The Trigger T^{R} dienophile is an eight-membered non-aromatic cyclic alkenylene group, preferably a cyclooctene group, and more preferably a trans-cyclooctene group. These eight-membered groups are herein collectively abbreviated as TCO.

In this invention, the TCO satisfies the following formula (1a):

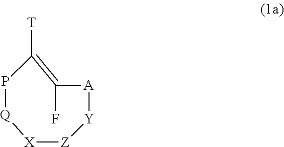

wherein A and P each independently are CR^{a}₂ or CR^{a}X^{D}, provided that at least one, and preferably not more than one, is CR^{a}X^{D}. X^{D} is ((O))ₚ-(L^{D})ₙ-(M^{M}), S—C(O)—) (L^{D})ₙ-(M^{M}), O—C(S)-(L^{D})ₙ-(M^{M}), S—S—C(S)-(L^{D})ₙ-(M^{M}), O—S(O)-(L^{D})ₙ-(M^{M}), wherein p=0 or 1. Preferably, X^{D} is (O—C(O))ₚ-(L^{D})ₙ-(M^{M}), where p=0 or 1, preferably 1, and n=0 or 1.

In an interesting embodiment, Y, Z, X, Q each independently are selected from the group consisting of CR^{a}₂, C═CR^{a}₂, C═O, C═S, C═NR^{b}, S, SO, SO₂, O, NR^{b}, and SiR^{c}₂, with at most three of Y, Z, X, and Q being selected from the group consisting of C═CR^{a}₂, C═O, C═S, and C═NR^{b}, wherein two R moieties together may form a ring, and with the proviso that no adjacent pairs of atoms are present selected from the group consisting of O—O, O—NR^{b}, S—NR^{b}, O—S, O—S(O), O—S(O)₂, and S—S, and such that Si is only adjacent to CR^{a}₂ or O.

In another interesting embodiment, one of the bonds PQ, QX, XZ, ZY, YA is part of a fused ring or consists of CR^{a}═CR^{a}, such that two exocyclic bonds are fixed in the same plane, and provided that PQ and YA are not part of an aromatic 5- or 6-membered ring, of a conjugated 7-membered ring, or of CR^{a}═CR^{a}; when not part of a fused ring P and A are independently CR^{a}₂ or CR^{a}X^{D}, provided that at least one, and preferably not more than one, is CR^{a}X^{D}; when part of a fused ring P and A are independently CR^{a} or CX^{D}, provided that at least one, and preferably not more than one, is CX^{D}; the remaining groups (Y,Z,X,Q) being independently from each other CR^{a}₂, C═CR^{a}₂, C═O, C═S, C═NR^{b}, S, SO, SO₂, O, NR^{b}, SiR^{c}₂, such that at most 1 group is C═CR^{a}₂, C═O, C═S, C═NR^{b}, and no adjacent pairs of atoms are present selected from the group consisting of O—O, O—NR^{b}, S—NR^{b}, O—S, O—S(O), O—S(O)₂, and S—S, and such that Si, if present, is adjacent to CR^{a}₂ or O, and the CR^{a}₂═CR^{a}₂ bond, if present, is adjacent to CR^{a}₂ or C═CR^{a}₂ groups;

In some embodiments fused rings are present that result in two exocyclic bonds being fixed in substantially the same plane. These are selected from fused 3-membered rings, fused 4-membered rings, fused bicyclic 7-membered rings, fused aromatic 5-membered rings, fused aromatic 6-membered rings, and fused planar conjugated 7-membered rings as defined below:

Fused 3-membered rings are:

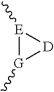

Therein E, G are part of the above mentioned 8-membered ring and can be fused to PQ, QP, QX, XQ, XZ, ZX, ZY, YZ, YA, AY, such that P, A are CR^{a} or CX^{D}, and such that CX^{D} can only be present in A and P.

E-G is CR^{a}—CR^{a} or CR^{a}—CX^{D}, and D is CR^{a}₂, C═O, C═S, C═NR^{b}, NR^{b}, O, S; or E-G is CR^{a}—N or CX^{D}—N, and D is CR^{a}₂, C═O, C═S, C═NR^{b}, NR^{b}O, or S.

Fused 4-membered rings are:

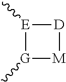

E-G is part of the above mentioned 8-membered ring and can be fused to PQ, QP, QX, XQ, XZ, ZX, ZY, YZ, YA, AY, such that P, A are C, CR^{a} or CX^{D}, and such that CX^{D} can only be present in A and P.

E, G are CR^{a}, CX^{D} or N, and D, M independently from each other are CR^{a}₂, C═O, C═S, C═NR^{b}, C═CR^{a}₂, S, SO, SO₂, O, NR^{b} but no adjacent O—O or S—S groups; or -D is C═CR^{a} and G is N, CR^{a}, CX^{D} and M is CR^{a}₂, S, SO, SO₂, O, NR^{b}; or E-D is C═N and G is N, CR^{a}, CX^{D} and M is CR^{a}₂, S, SO, SO₂, O; or D-M is CR^{a}═CR^{a} and E, G each independently are CR^{a}, CX^{D} or N; or D-M is CR^{a}═N and E is CR^{a}, CX^{D}, N, and G is CR^{a} or CX^{D}; or E is C, G is CR^{a}, CX^{D} or N, and D, M are CR^{a}₂, S, SO, SO₂, O, NR^{b}, or at most one of C═O, C═S, C═NR^{b}, C═CR^{a}₂, but no adjacent O—O or S—S groups; or E and G are C, and D and M independently from each other are CR^{a}₂, S, SO, SO₂, O, NR^{b} but no adjacent O—O, or S—S groups.

Fused bicyclic 7-membered rings are:

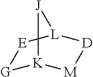

E-G is part of the above mentioned 8-membered ring and can be fused to PQ, QP, QX, XQ, XZ, ZX, ZY, YZ, YA, AY, such that P, A are C, CR^{a} or CX^{D}, and such that CX^{D} can only be present in A and P;

E,G are C, CR^{a}, CX^{D} or N; K, L are CR^{a}; D,M form a CR^{a}═CR^{a} or CR^{a}═N, or D,M independently from each other are CR^{a}₂, C═O, C═S, C═NR^{b}, C═CR^{a}₂, S, SO, SO₂, O, NR^{b} but no adjacent O—O, S—S, N—S groups; J is CR^{a}₂, C═O, C═S, C═NR^{b}, C═CR^{a}₂, S, SO, SO₂, O, NR^{b}; at most 2 N groups; or E,G are C, CR^{a}, CX^{D}; K is N and L is CR^{a}; D,M form a CR^{a}═CR^{a} bond or D,M independently from each other are CR^{a}₂, C═O, C═S, C═NR^{b}, C═CR^{a}₂, NR^{b} but no adjacent O—O, S—S, N—S groups; J is CR^{a}₂, C═O, C═S, C═NR^{b}, C═CR^{a}₂, S, SO, SO₂, O, NR^{b}; at most 2 N groups; or E,G are C, CR^{a}, CX^{D}; K and L are N; D,M, J independently from each other are CR^{a}₂, C═O, C═S, C═NR^{b}, C═CR^{a}₂ groups;

Fused aromatic 5-membered rings are

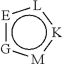

E, G are part of the above mentioned 8-membered ring and can be fused to QX, XQ, XZ, ZX, ZY, YZ.

E and G are C; one of the groups L, K, or M are O, NR^{b}, S and the remaining two groups are independently from each other CR^{a} or N; or E is C and G is N; L, K, M are independently from each other CR^{a} or N.

Fused aromatic 6-membered rings are:

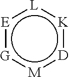

E, G are part of the above mentioned 8-membered ring and can be fused to QX, XQ, XZ, ZX, ZY, YZ.

E,G is C; L, K, D, M are independently from each other CR^{a} or N.

Fused planar conjugated 7-membered rings are

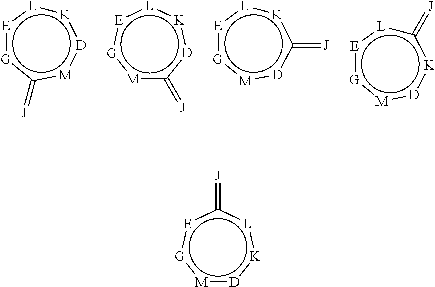

E, G are part of the above mentioned 8-membered ring and can be fused to QX, XQ, XZ, ZX, ZY, YZ; E,G is C; L, K, D, M are CR^{a}; J is S, O, CR^{a}₂, NR^{b}. (L^{D})ₙ is an optional linker, with n=0 or 1, preferably linked to T^{R} via S, N, NH, or O, wherein these atoms are part of the linker, which may consist of multiple units arranged linearly and/or branched. M^{M} is a masking moiety M^{M}, preferably linked via S, N, NH, or O, wherein these atoms are part of M^{M}. T, F each independently denotes H, or a substituent selected from the group consisting of alkyl, F, Cl, Br, or I.

Without wishing to be bound by theory, the inventors believe that in the foregoing embodiments, the rDA reaction results in a cascade-mediated release or elimination (i.e. cascade mechanism) of the Masking Moiety.

In several alternative embodiments, with reference to formula (1a), said release or elimination is believed to be mediated by a strain release mechanism.

Therein, in Embodiment 1, one of the bonds PQ, QP, QX, XQ, XZ, ZX, ZY, YZ, YA, AY consists of —CR^{a}X^{D}—CR^{a}Y_{D}—, the remaining groups (from A,Y,Z,X,Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O.

X^{D} is O—C(O)-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), NR^{d}—C(O)-(L^{D})ₙ-(M^{M}), NR^{d}—C(S)-(L^{D})ₙ-(M^{M}), and then Y^{D} is NHR^{d}, OH, SH; or X^{D} is C(O)-(L^{D})ₙ-(M^{M}), C(S)-(L^{D})ₙ-(M^{M}); and then Y^{D} is CR^{d}₂NHR^{d}, CR^{d}₂OH, CR^{d}₂SH, NH—NH₂, O—NH₂, NH—OH.

Preferably X^{D} is NR^{d}—C(O)-(L^{D})ₙ-(M^{M}), and Y^{D} is NHR^{d}.

In this Embodiment 1, the X^{D} and Y^{D} groups may be positioned cis or trans relative to each other, where depending on the positions on the TCO, cis or trans are preferred: if PQ, QP, AY or YA is —CR^{a}X^{D}—CR^{a}Y^{D}—, then X^{D} and Y^{D} are preferably positioned trans relative to each other; if ZX or XZ is —CR^{a}X^{D}—CR^{a}Y^{D}—, then X^{D} and Y^{D} are preferably positioned cis relative to each other.

In Embodiment 2, A is CR^{a}X^{D} and Z is CR^{a}Y^{D}, or Z is CR^{a}X^{D} and A is CR^{a}Y^{D}, or P is CR^{a}X^{D} and X is CR^{a}Y^{D}, or X is CR^{a}X^{D} and P is CR^{a}Y^{D}, such that X^{D} and Y^{D} are positioned in a trans conformation with respect to one another; the remaining groups (from A,Y, Z, X, Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O; X^{D} is O—C(O)-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), NR^{d}—C(O)-(L^{D})ₙ-(M^{M}), NR^{d}—C(S)-(L^{D})ₙ-(M^{M}), and then Y^{D} is NHR^{d}, OH, SH, CR^{d}₂NHR^{d}, CR^{d}₂OH, CR^{d}₂SH, N H—NH₂, O—NH₂, NH—OH; or X^{D} is CR^{d}₂—O—C(O)-(L^{D})ₙ-(M^{M}), CR^{d}₂-S—C(O)-(L^{D})ₙ-(M^{M}), CR^{d}₂—O—C(S)-(L^{D})ₙ-(M^{M}), CR^{d}₂—S—C(S)-(L^{D})ₙ-(M^{M}), CR^{d}₂-NR^{d}—C(O)-(L^{D})ₙ-(M^{M}), CR^{d}₂—NR^{d}—C(S)-(L^{D})ₙ-(M^{M}); and then Y^{D} is NHR^{d}, OH, SH; or X^{D} is C(O)-(L^{D})ₙ-(M^{M}), C(S)-(L^{D})ₙ-(M^{M}); and then Y^{D} is CR^{d}₂NHR^{d}, CR^{d}₂OH, CR^{d}2 SH, NH—NH₂, O—NH₂, NH—OH.

Preferably X^{D} is NR^{d}—C(O)-(L^{D})ₙ-(M^{M}), and Y^{D} is NHR^{d}.

In Embodiment 3, A is CR^{a}Y^{D} and one of P, Q, X, Z is CR^{a}X^{D}, or P is CR^{a}Y^{D} and one of A, Y, Z, X is CR^{a}X^{D}, or Y is CR^{a}Y^{D} and X or P is CR^{a}X^{D}, or Q is CR^{a}Y^{D} and Z or A is CR^{a}X^{D}, or either Z or X is CR^{a}Y^{D} and A or P is CR^{a}X^{D}, such that X^{D} and Y^{D} are positioned in a trans conformation with respect to one another; the remaining groups (from A,Y, Z, X, Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O.

X^{D} is (O—C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}); Y^{D} is CR^{d}₂NHR^{d}, CR^{d}₂OH, CR^{d}₂SH, NH—NH₂, O—NH₂, NH—OH; p=0 or 1.

Preferably X^{D} is (O—C(O))ₚ-(L^{D})ₙ-(M^{M}), with p=1, and Y^{D} is CR^{d}₂NHR^{d}.

In Embodiment 4, P is CR^{a}Y^{D} and Y is CR^{a}X^{D}, or A is CR^{a}Y^{D} and Q is CR^{a}X^{D}, or Q is CR^{a}Y^{D} and A is CR^{a}X^{D}, or Y is CR^{a}Y^{D} and P is CR^{a}X^{D}, such that X^{D} and Y^{D} are positioned in a trans conformation with respect to one another; the remaining groups (from A,Y, Z, X, Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O.

X^{D} is (O—C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}); Y^{D} is NHR^{d}, OH, SH; p=0 or 1.

Preferably X^{D} is (O—C(O))ₚ-(L^{D})ₙ-(M^{M}), with p=1, and Y^{D} is NHR^{d}.

In Embodiment 5, Y is Y^{D} and P is CR^{a}X^{D}, or Q is Y^{D} and A is CR^{a}X^{D}; the remaining groups (from A,Y, Z, X, Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O.

X^{D} is (O—C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), NR^{d}—C(O)-(L^{D})ₙ-(M^{M}), NR^{d}—C(S)-(L^{D})ₙ-(M^{M}), C(O)-(L^{D})ₙ-(M^{M}), C(S)-(L^{D})ₙ-(M^{M}); Y^{D} is NH; p=0 or 1.

Preferably X^{D} is NR^{d}—C(O)-(L^{D})ₙ-(M^{M}) or (O—C(O))ₚ-(L^{D})ₙ-(M^{M}), with p=0 or 1.

In Embodiment 6, Y is Y^{D} and P or Q is X^{D}, or Q is Y^{D} and A or Y is V; the remaining groups (from A,Y, Z, X, Q,P) being independently from each other CR^{a}₂, S, O, SiR^{c}₂, such that P and A are CR^{a}₂, and no adjacent pairs of atoms are present selected from the group consisting of O—O, O—S, and S—S, and such that Si, if present, is adjacent to CR^{a}₂ or O.

X^{D} is N—C(O)-(L^{D})ₙ-(M^{M}), N—C(S)-(L^{D})ₙ-(M^{M}); Y^{D} is NH; Preferably X^{D} is N—C(O)-(L^{D})ₙ-(M^{M}).

Also herein, (L^{D})ₙ is an optional linker, with n=0 or 1, preferably linked to T^{R} via S, N, NH, or O, wherein these atoms are part of the linker, which may consist of multiple units arranged linearly and/or branched. M^{M} is masking moiety, preferably linked via S, N, NH, or O, wherein these atoms are part of M^{M}.

T, F each independently denotes H, or a substituent selected from the group consisting of alkyl, F, Cl, Br, or I.

It is preferred that when M^{M} is bound to T^{R} or L^{D} via NH, this NH is a primary amine (—NH₂) residue from M^{M}, and when M^{M} is bound via N, this N is a secondary amine (—NH—) residue from M^{M}. Similarly, it is preferred that when M^{M} is bound via O or S, said O or S are, respectively, a hydroxyl (—OH) residue or a sulfhydryl (—SH) residue from M^{M}.

It is further preferred that said S, N, NH, or O moieties comprised in M^{M} are bound to an aliphatic or aromatic carbon of M^{M}.

It is preferred that when L^{D} is bound to T^{R} via NH, this NH is a primary amine (—NH₂) residue from L^{D}, and when L^{D} is bound via N, this N is a secondary amine (—NH—) residue from L^{D}. Similarly, it is preferred that when L^{D} is bound via O or S, said O or S are, respectively, a hydroxyl (—OH) residue or a sulfhydryl (—SH) residue from L^{D}.

It is further preferred that said S, N, NH, or O moieties comprised in L^{D} are bound to an aliphatic or aromatic carbon of L^{D}.

Where reference is made in the invention to a linker L^{D} this can be self-immolative or not, or a combination thereof, and which may consist of multiple self-immolative units. It will be understood that if L^{D} is not self-immolative, the linker equals a spacer S^{P}.

By way of further clarification, if p=0 and n═O, the species M^{M} directly constitutes the leaving group of the elimination reaction, and if p=0 and n=1, the self-immolative linker constitutes the leaving group of the elimination. The position and ways of attachment of linkers L^{D} and moieties M^{M} are known to the skilled person (see for example Papot et al, Anti-Cancer Agents in Medicinal Chemistry, 2008, 8, 618-637). Nevertheless, typical but non-limiting examples of self-immolative linkers L^{D} are benzyl-derivatives, such as those drawn below. On the right, an example of a self-immolative linker with multiple units is shown; this linker will degrade not only into CO₂ and one unit of 4-aminobenzyl alcohol, but also into one 1,3-dimethylimidazolidin-2-one unit.

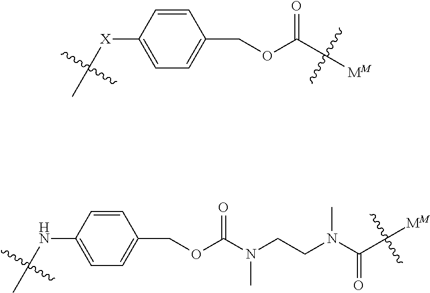

X═O or S or NH or NR with R=alkyl or aryl

By substituting the benzyl groups of aforementioned self-immolative linkers L^{D}, preferably on the 2- and/or 6-position, it may be possible to tune the rate of release of the species M^{M}, caused by either steric and/or electronic effects on the intramolecular elimination reaction. Synthetic procedures to prepare such substituted benzyl-derivatives are known to the skilled person (see for example Greenwald et al, J. Med. Chem., 1999, 42, 3657-3667 and Thornthwaite et al, Polym. Chem., 2011, 2, 773-790).

In a preferred embodiment, the TCO of formula (1a) is an all-carbon ring. In another preferred embodiment, the TCO of formula (1a) is a heterocyclic carbon ring, having of one to two oxygen atoms in the ring, and preferably a single oxygen atom.

Each R^{a} as above-indicated can independently be H, alkyl, aryl, OR′, SR′, S(═O)R′″, S(═O)₂R′″, S(═O)₂NR′R″, Si—R″, Si—O—R′″, OC(═O)R′″, SC(═O)R′″, OC(═S)R′″, SC(═S)R′″, F, Cl, Br, I, N₃, SO₂H, SO₃H, SO₄H, POSH, POOH, NO, NO₂, CN, OCN, SCN, NCO, NCS, CF₃, CF₂—R′, NR′R″, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R″, NR′C(═O)S—R″, NR′C(═S)S—R′″, OC(═O)NR′—R′″, SC(═O)NR′—R′″, OC(═S)NR′—R′″, SC(═S)NR′—R′″, NR′C(═O)NR″—R″, NR′C(═S)NR″—R″, CR′NR″, with each R′ and each R″ independently being H, aryl or alkyl and R′″ independently being aryl or alkyl; Each R^{d} as above indicated is independently selected from the group consisting of H, alkyl, aryl, O-aryl, O-alkyl, OH, C(═O)NR′R″ with R′ and R″ each independently being H, aryl or alkyl, R′CO-alkyl with R′ being H, alkyl, and aryl; Each R^{c} as above indicated is independently selected from the group consisting of H, alkyl, aryl, O-alkyl, O-aryl, OH; Each R^{d} as above indicated is independently selected from H, C₁₋₆ alkyl and C₁₋₆ aryl; wherein two or more R^{a,b,c,d} moieties together may form a ring.

Preferably, each R^{a} is selected independently from the group consisting of H, alkyl, O-alkyl, O-aryl, OH, C(═O)O—R′, C(═O)NR′R″, NR′C(═O)—R′″, with R′ and R″ each independently being H, aryl or alkyl, and with R′″ independently being alkyl or aryl.

In all of the above embodiments, one of A, P, Q, Y, X, and Z, or the substituents or fused rings of which they are part, or the self-immolative linker L^{D}, is bound, optionally via a spacer or spacers S^{P}, to the Drug D^{D}. Synthetic procedures to prepare D^{D} conjugates with T^{R} are known to the skilled person.

The synthesis of TCO's as described above is well available to the skilled person. This expressly also holds for TCO's having one or more heteroatoms in the strained cycloalkene rings. References in this regard include Cere et al. Journal of Organic Chemistry 1980, 45, 261 and Prevost et al. Journal of the American Chemical Society 2009, 131, 14182.

In a preferred embodiment, the trans-cyclooctene moiety satisfies formula (1 b):

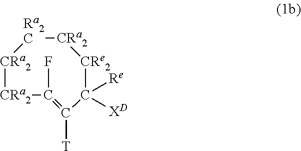

wherein, in addition to the optional presence of at most two exocyclic bonds fixed in the same plane, each R^{a} independently denotes H, or, in at most four instances, a substituent selected from the group consisting of alkyl, aryl, OR′, SR′, S(═O)R′″, S(═O)₂R′″, S(═O)₂NR′R″, Si—R″, Si—O—R′″, OC(═O)R′″, SC(═O)R′″, OC(═S)R′″, SC(═S)R′″, F, Cl, Br, I, N₃, SO₂H, SO₃H, SO₄H, POSH, POOH, NO, NO₂, CN, OCN, SCN, NCO, NCS, CF₃, CF₂—R′, NR′R″, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R′″, NR′C(═O)S—R′″, NR′C(═S)S—R′″, OC(═O)NR′—R′″, SC(═O)NR′—R′″, OC(═S)NR′—R′″, SC(═S)NR′—R′″, NR′C(═O)NR″—R″, NR′C(═S)NR″—R″, CR′NR″, with each R′ and each R″ independently being H, aryl or alkyl and R′″ independently being aryl or alkyl; Each R^{e} as above indicated is independently selected from the group consisting of H, alkyl, aryl, OR′, SR′, S(═O)R′″, S(═O)₂R′″, Si—R″, Si—O—R″, OC(═O)R′″, SC(═O)R′″, OC(═S)R′″, SC(═S)R′″, F, Cl, Br, I, N₃, SO₂H, SO₃H, POSH, NO, NO₂, CN, CF₃, CF₂—R′, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R′″, NR′C(═O)S—R′″, NR′C(═S)S—R′″, NR′C(═O)NR″—R″, NR′C(═S)NR″—R″, CR′NR″, with each R′ and each R″ independently being H, aryl or alkyl and R′″ independently being aryl or alkyl; wherein two R^{a,e} moieties together may form a ring; wherein one R^{a,e} or the self-immolative linker L^{D}, is bound, optionally via a spacer or spacers S^{P}, to the species D^{D}, and wherein T and F each independently denote H, or a substituent selected from the group consisting of alkyl, F, Cl, Br, and I, and X^{D} is (0-C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), O—S(O)-(L^{D})ₙ-(M^{M}), wherein p=0 or 1. Preferably, X^{D} is (O—C(O))ₚ-(L^{D})ₙ-(M^{M}), where p=0 or 1, preferably 1, and n=0 or 1.

Preferably, each R^{a} and each Re is selected independently from the group consisting of H, alkyl, O-alkyl, O-aryl, OH, C(═O)O—R′, C(═O)NR′R″, NR′C(═O)—R′″, with R′ and R″ each independently being H, aryl or alkyl, and with R′″ independently being alkyl or aryl.

In the foregoing dienophiles, it is preferred that the at least two exocyclic bonds fixed in the same plane are selected from the group consisting of (a) the single bonds of a fused cyclobutyl ring, (b) the hybridized bonds of a fused aromatic ring, (c) an exocyclic double bond to an oxygen, and (d) an exocyclic double bond to a carbon.

The TCO, containing one or two X^{D} moieties, may consist of multiple isomers, also comprising the equatorial vs. axial positioning of substituents, such as X^{D}, on the TCO. In this respect, reference is made to Whitham et al. J. Chem. Soc. (C), 1971, 883-896, describing the synthesis and characterization of the equatorial and axial isomers of trans-cyclo-oct-2-en-ol, identified as (1RS, 2RS) and (1SR, 2RS), respectively. In these isomers the OH substituent is either in the equatorial or axial position.

In a preferred embodiment, with reference to formula (1a), for structures where the substituents of A and/or P, such as X^{D} and Y^{D}, can be either in the axial or the equatorial position, the substituent is in the axial position.

Preferred dienophiles, which are optimally selected for M^{M} release believed to proceed via a cascade elimination mechanism, are selected from the following structures:

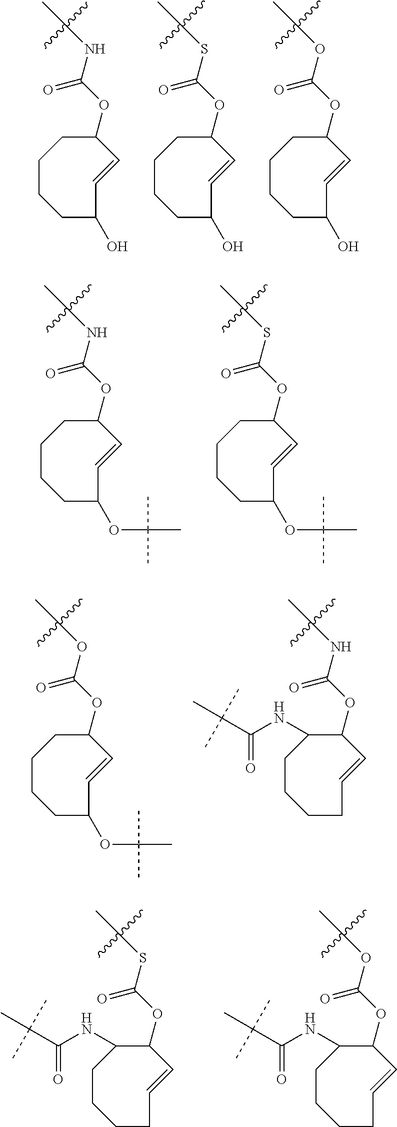

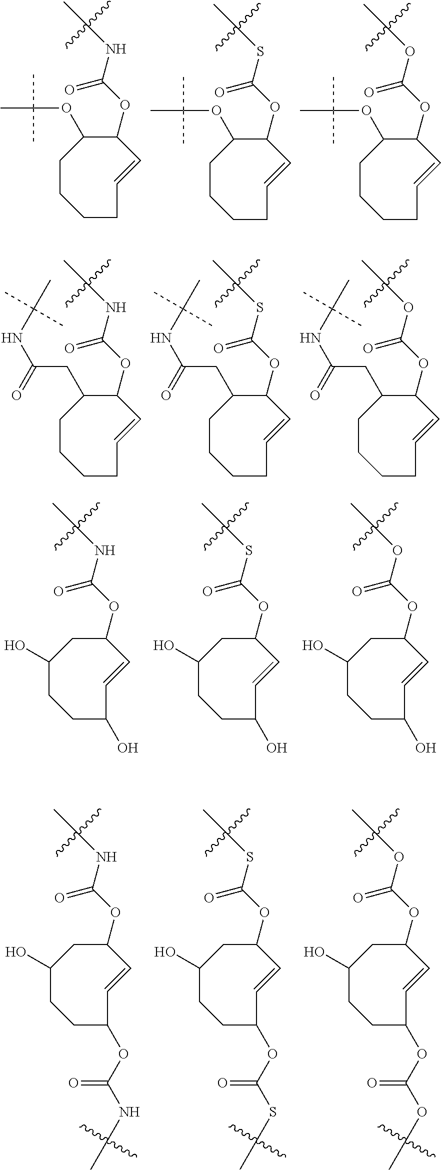

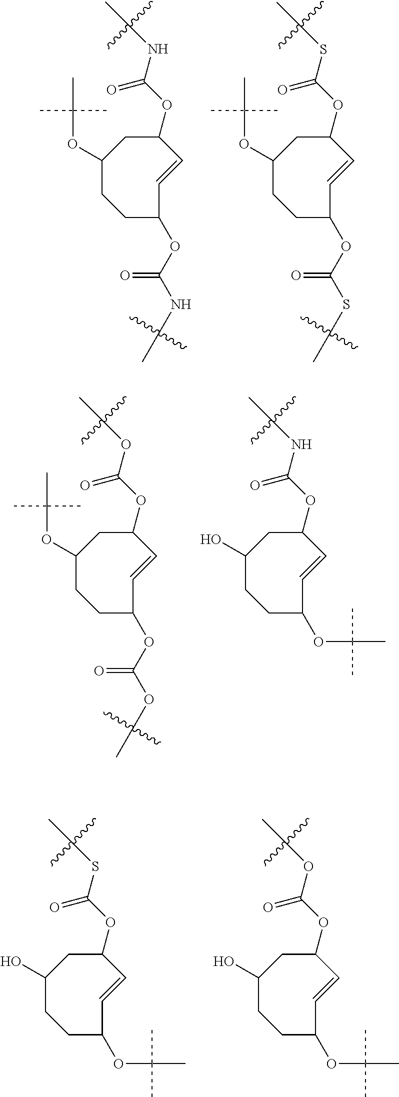

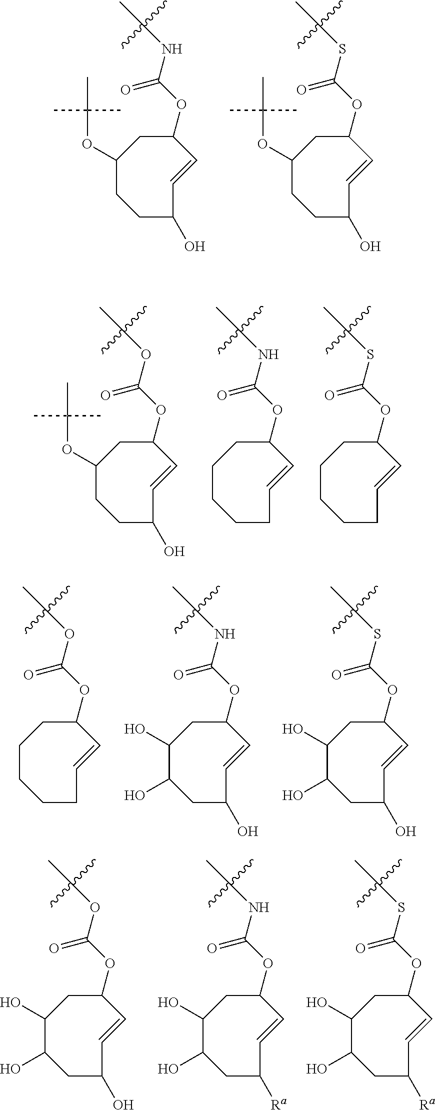

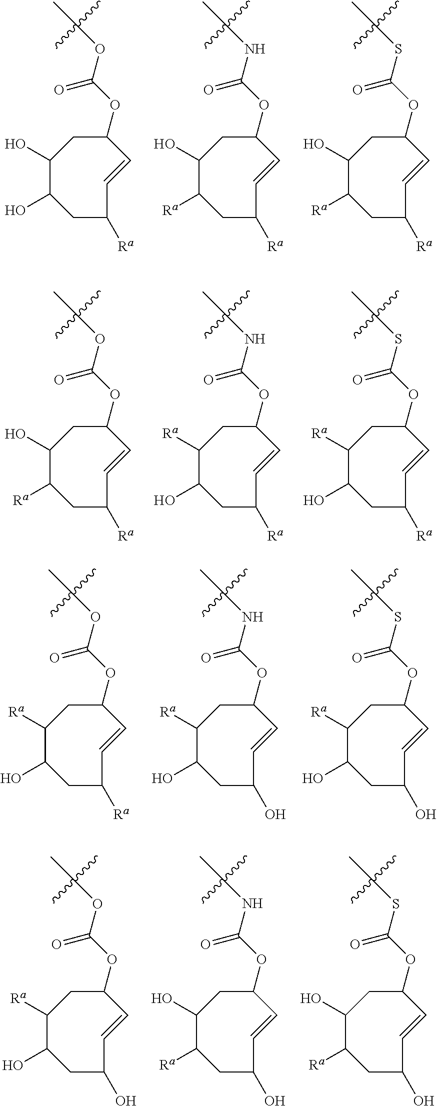

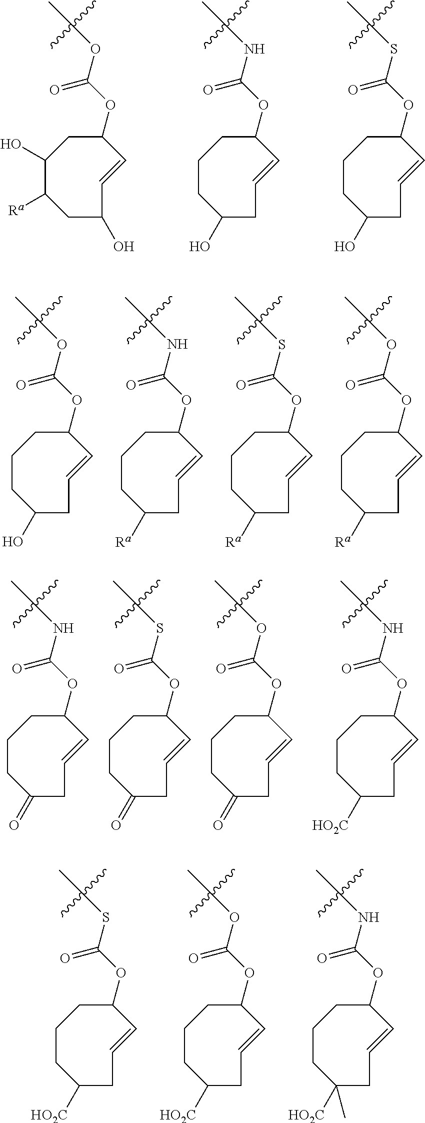

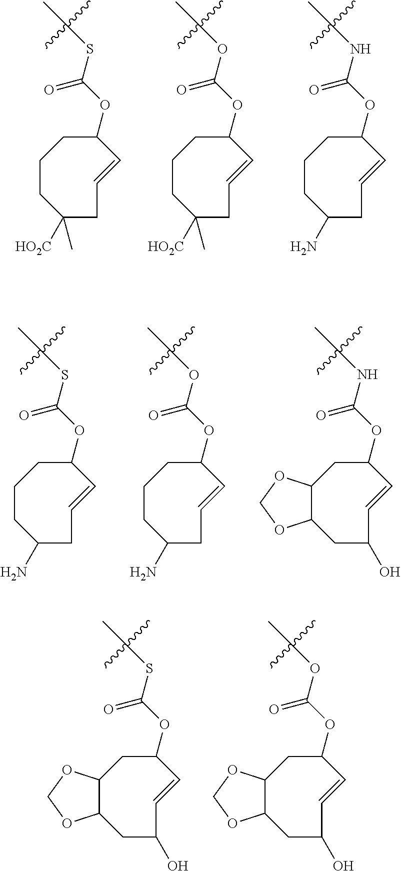

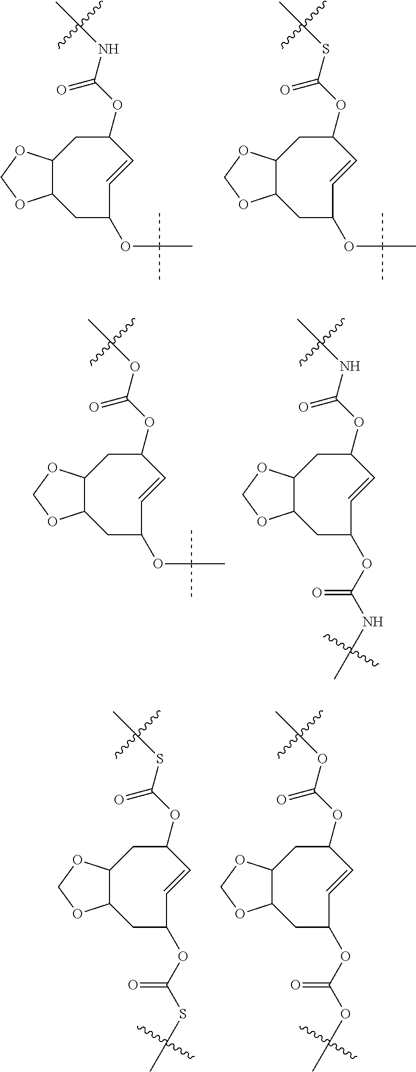

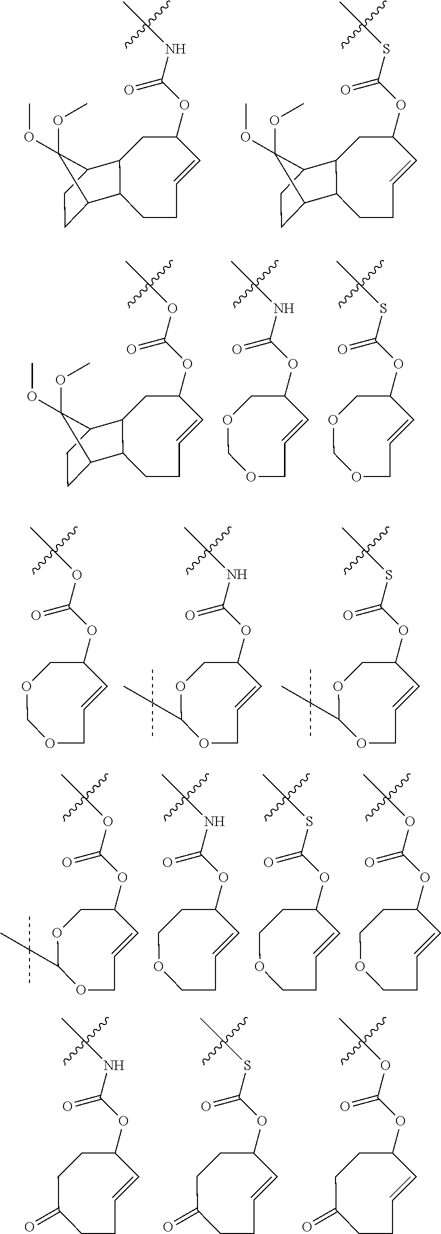

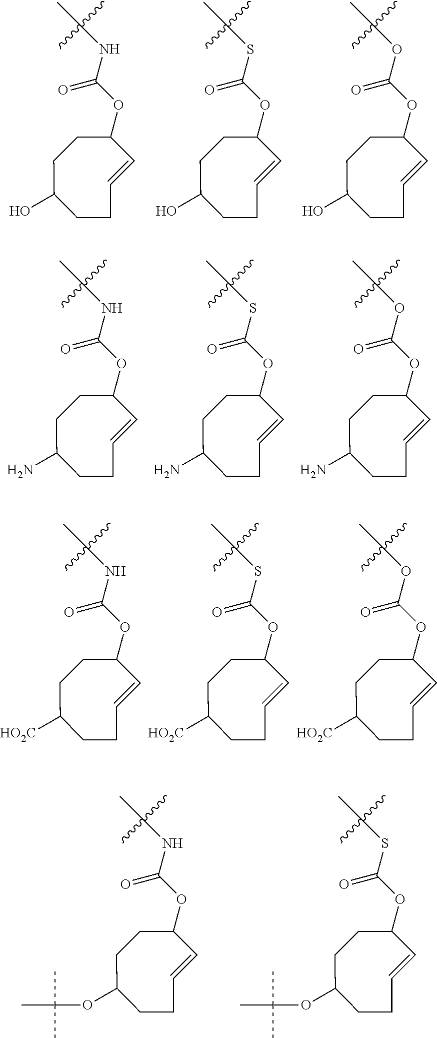

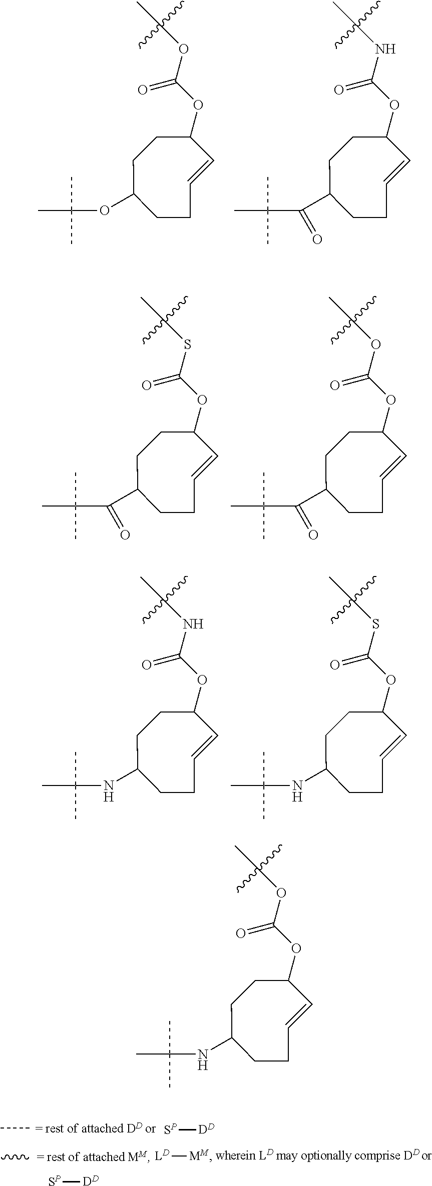

Preferred dienophiles, which are optimally selected for M^{M} release believed to proceed via a strain release mechanism, are selected from the following structures:

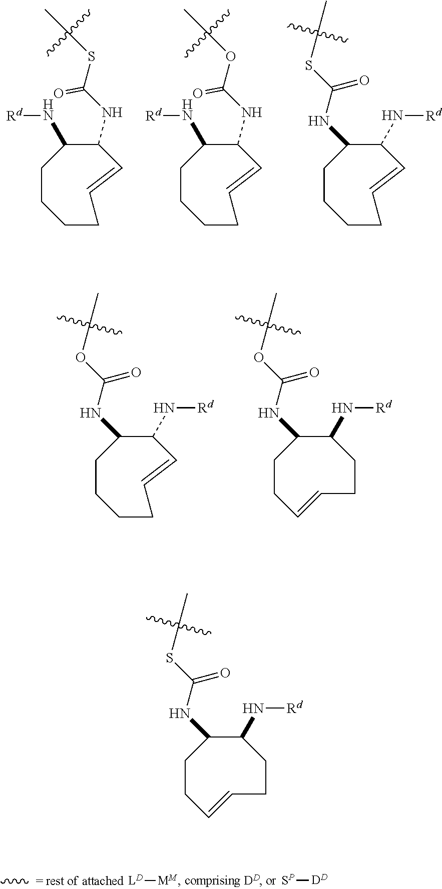

In a further preferred embodiment, the dienophile is a compound selected from the following structures:

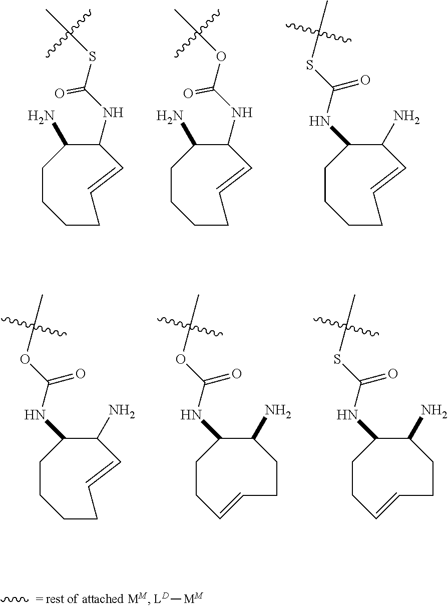

In alternative embodiments, the dienophile is a compound selected from the following structures:

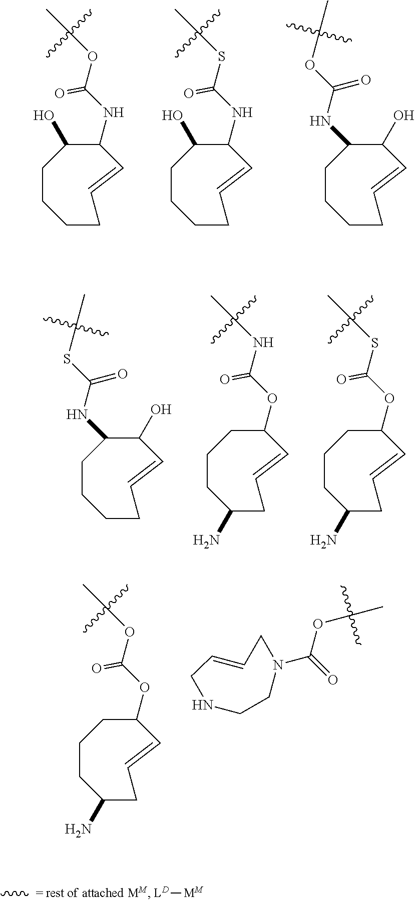

### Use of TCO as a Carrier

The invention also pertains to the use of a trans-cyclooctene satisfying formula (1a), in all its embodiments, as a carrier for a therapeutic compound. The trans-cyclooctene is to be read as a TCO in a broad sense, as discussed above, preferably an all-carbon ring or including one or two hetero-atoms. A therapeutic compound is a drug or other compound or moiety intended to have therapeutic application. The use of TCO as a carrier according to this aspect of the invention does not relate to the therapeutic activity of the therapeutic compound. In fact, also if the therapeutic compound is a drug substance intended to be developed as a drug, many of which will fail in practice, the application of TCO as a carrier still is useful in testing the drug. In this sense, the TCO in its capacity of a carrier is to be regarded in the same manner as a pharmaceutical excipient, serving as a carrier when introducing a drug into a subject.

The use of a TCO as a carrier has the benefit that it enables the administration, to a subject, of a drug carried by a moiety that is open to a bioorthogonal reaction, with a diene, particularly a tetrazine. This provides a powerful tool not only to affect the fate of the drug carried into the body, but also to follow its fate (e.g. by allowing a labeled diene to react with it), or to change its fate (e.g. by allowing pK modifying agents to bind with it). This is all based on the possibility to let a diene react with the TCO in the above-discussed rDA reaction. The carrier is preferably reacted with an Activator as discussed below, so as to provoke the release of the M^{M} from the TCO, as amply discussed herein.

### Activator Induced Release

The Activator comprises a Bio-orthogonal Reactive Group, wherein this Bio-orthogonal Reactive Group of the Activator is a diene. This diene reacts with the other Bio-orthogonal Reactive Group, the Trigger, and that is a dienophile (vide supra). The diene of the Activator is selected so as to be capable of reacting with the dienophile of the Trigger by undergoing a Diels-Alder cycloaddition followed by a retro Diels-Alder reaction, giving the Retro Diels-Alder adduct. This intermediate adduct then releases the Masking Moiety or Moieties, where this release can be caused by various circumstances or conditions that relate to the specific molecular structure of the retro Diels-Alder adduct. Without wishing to be bound by theory, the inventors believe that the Activator is selected such as to provoke Masking Moiety release via an elimination or cascade elimination (via an intramolecular elimination reaction within the Retro Diels-Alder adduct). This elimination reaction can be a simple one step reaction, or it can be a multiple step reaction that involves one or more intermediate structures. These intermediates may be stable for some time or may immediately degrade to the thermodynamic end product or to the next intermediate structure. When several steps are involved, one can speak of a cascade reaction. In any case, whether it be a simple or a cascade process, the result of the elimination reaction is that the Masking Moiety gets released from the retro Diels-Alder adduct. Without wishing to be bound by theory, the design of both components (i.e. the diene Activator and the dienophile Trigger) is such that the distribution of electrons within the retro Diels-Alder adduct is unfavorable, so that a rearrangement of these electrons must occur. This situation initiates the intramolecular (cascade) elimination reaction to take place, and it therefore induces the release of the Masking Moiety or Masking Moieties. Occurrence of the elimination reaction in and Masking Moiety release from the Prodrug is not efficient or cannot take place prior to the Retro Diels-Alder reaction, as the Prodrug itself is relatively stable as such. Elimination can only take place after the Activator and the Prodrug have reacted and have been assembled in the retro Diels-Alder adduct.

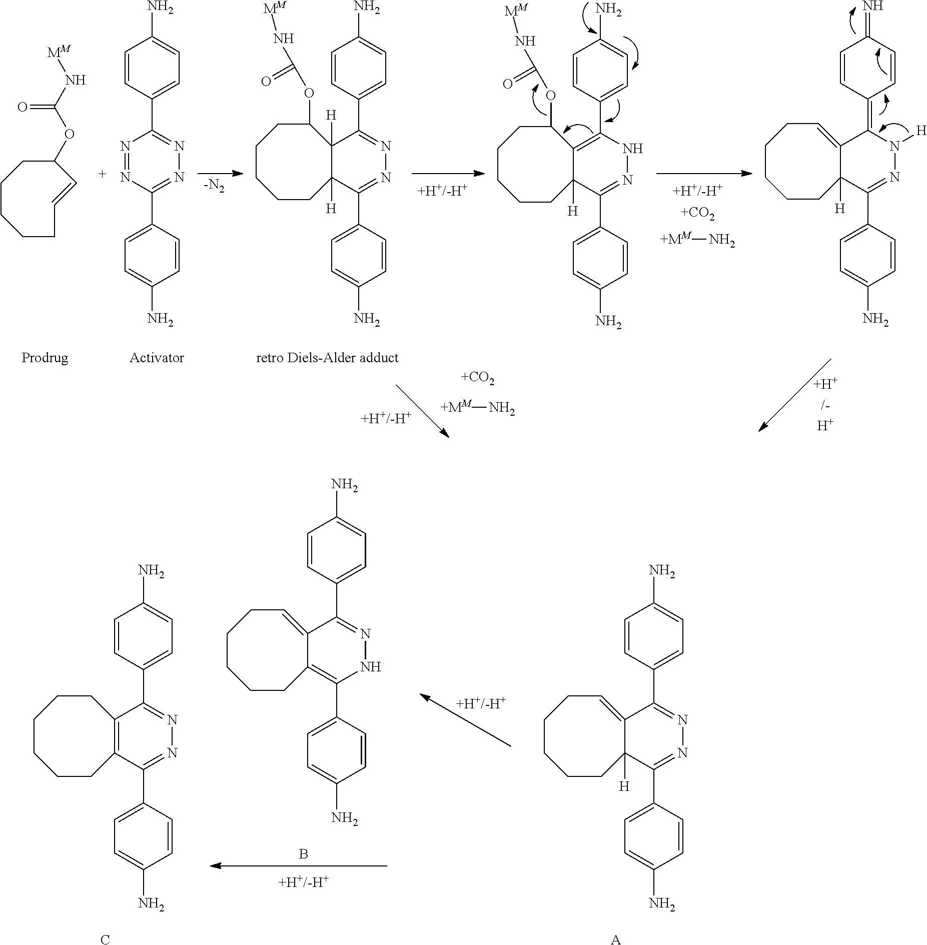

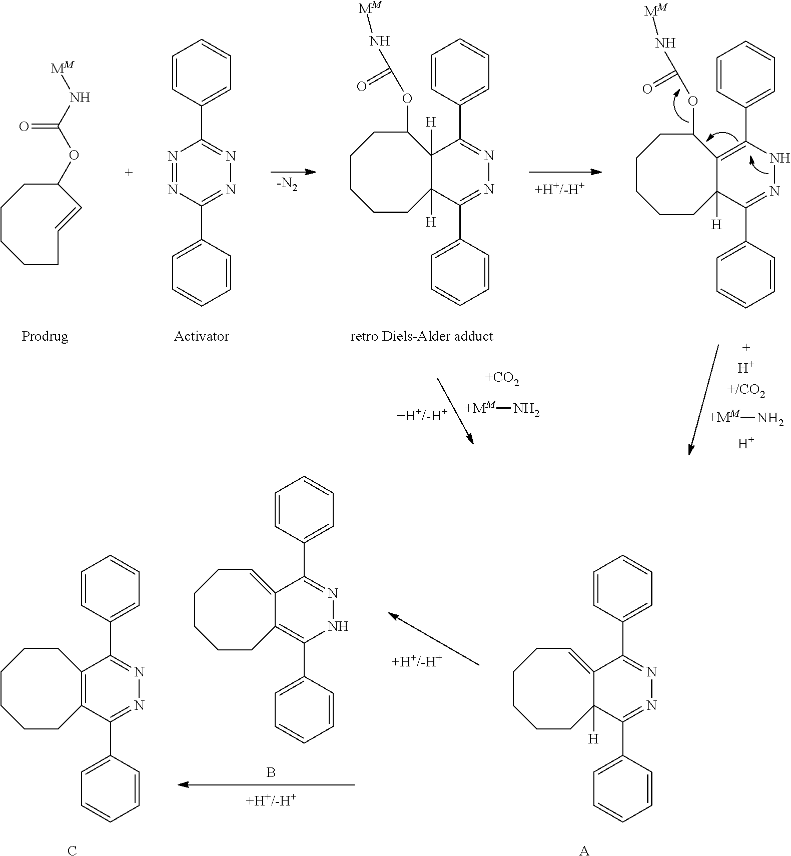

Without wishing to be bound by theory, the above two examples illustrate how the unfavorable distribution of electrons within the retro Diels-Alder adduct can be relieved by an elimination reaction, thereby releasing the Masking Moiety. In one scenario, the elimination process produces end product A, where this product has a conjugation of double bonds that was not present in the retro Diels-Alder adduct yet. Species A may tautomerize to end product B, or may rearrange to form end product C. Then, the non-aromatic dihydro pyridazine ring in the retro Diels-Alder adduct has been converted to the aromatic pyridazine ring in the end product C. The skilled person will understand that the distribution of electrons in the retro Diels-Alder adduct is generally unfavorable relative to the distribution of the electrons in the end products, either species A or B or C. Thus, the formation of a species stabler than the retro Diels-Alder adduct is the driving force for the (cascade) elimination reaction. In any case, and in whatever way the process is viewed, the Masking Moiety species (here the amine ‘M^{M}-NH₂’) is effectively expelled from the retro Diels-Alder adduct, while it does not get expelled from the Prodrug alone.

Below scheme depicts a possible alternative release mechanism for the cascade elimination, in addition to the two discussed above. Without wishing to be bound by theory, the below examples illustrates how the unfavorable distribution of electrons within the retro Diels-Alder adduct may be relieved by an elimination reaction, thereby releasing the Masking Moiety. This process may evolve via various tauromerisations that are all equilibria. Here, the rDA reaction produces tautomers A and B, which can interchange into one and other. Tautomer B can lead to the elimination into product C and thereafter into D.

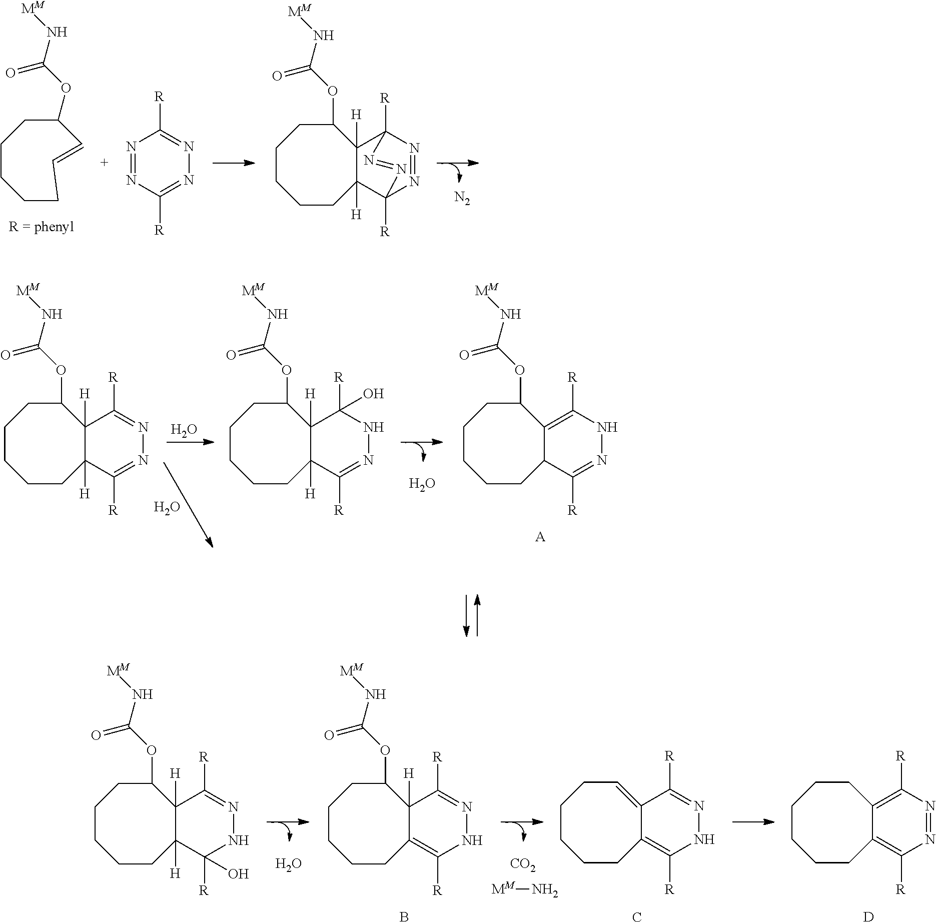

As discussed above, in this invention, the releasing effect of the rDA reaction is, in one embodiment, caused by an intramolecular cyclization/elimination reaction within the part of the Retro Diels-Alder adduct that originates from the TCO dienophile. A nucleophilic site present on the TCO (i.e. the dienophile, particularly from the Y^{D} group in this Trigger, vide supra) reacts with an electrophilic site on the same TCO (particularly from the X^{D} group in this Trigger, vide supra) after this TCO reacts with the Activator to form an rDA adduct. The part of the rDA adduct that originates from the TCO, i.e. the eight membered ring of the rDA adduct, has a different conformation and has an increased conformational freedom compared to the eight membered ring in the TCO prior to the rDA reaction, allowing the nucleophilic reaction to occur, thereby releasing the M^{M} as a leaving group. The intramolecular cyclization/elimination reaction takes place, as the nucleophilic site and the electrophilic site have been brought together in close proximity within the Retro Diels-Alder adduct, and produce a favorable structure with a low strain. Additionally, the formation of the cyclic structure may also be a driving force for the intramolecular reaction to take place, and thus may also contribute to an effective release of the leaving group, i.e. release of the Masking Moiety. Reaction between the nucleophilic site and the electrophilic site does not take place or is relatively inefficient prior to the Retro Diels-Alder reaction, as both sites are positioned unfavorably for such a reaction, due to the relatively rigid, conformationally restrained TCO ring. The Prodrug itself is relatively stable as such and elimination is favored only after the Activator and the Prodrug have reacted and have been assembled in a retro Diels-Alder adduct that is subject to intramolecular reaction. In a preferred embodiment the TCO ring is in the crown conformation. The example below illustrates the release mechanism pertaining to this invention.

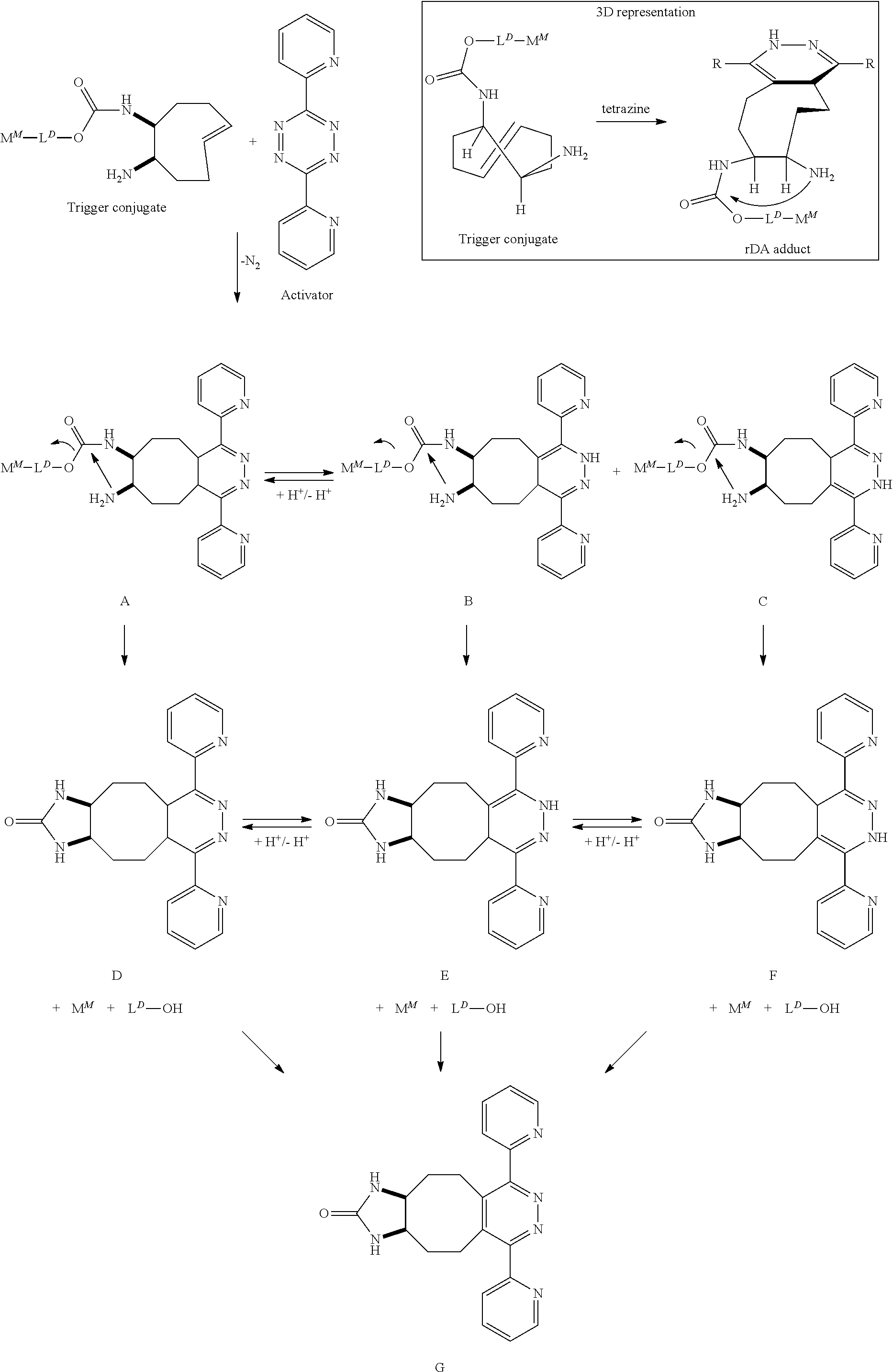

The above example illustrates how the intramolecular cyclization/elimination reaction within the retro Diels-Alder adduct can result in release of a Masking Moiety. The rDA reaction produces A, which may tautomerize to product B and C. Structures B and C may also tautomerize to one another (not shown). rDA products A, B, and C may intramolecularly cyclize, releasing the bound moiety, and affording structures D, E, and F, which optionally may oxidise to form product G. As the tautomerization of A into B and C in water is very fast (in the order of seconds) it is the inventors' belief, that release occurs predominantly from structures B and C. It may also be possible that the nucleophilic site assists in expelling the M^{M} species by a nucleophilic attack on the electrophilic site with subsequent release, but without actually forming a (stable) cyclic structure. In this case, no ring structure is formed and the nucleophilic site remains intact, for example because the ring structure is short lived and unstable and breaks down with reformation of the nucleophilic site.

Without wishing to be bound by theory, the above example illustrates how the conformational restriction and the resulting unfavorable positioning of the nucleophilic and electrophilic site in the TCO trigger is relieved following rDA adduct formation, leading to an elimination/cyclization reaction and release.

With respect to the nucleophilic site on the TCO, one has to consider that the site must be able to act as a nucleophile under conditions that may exist inside the (human) body, so for example at physiological conditions where the pH=ca. 7.4, or for example at conditions that prevail in malignant tissue where pH-values may be lower than 7.4. Preferred nucleophiles are amine, thiol or alcohol groups, as these are generally most nucleophilic in nature and therefore most effective.

### The Combination of and Reaction Between the TCO-Trigger and the Activator

It should be noted that in cases of release of amine functional M^{M} species these can be e.g. primary or secondary amine, aniline, imidazole or pyrrole type of moieties, so that the M^{M} is varying in leaving group character. Release of M^{M} with other functionalities may also be possible (e.g. thiol functinalized M^{M}), in case corresponding hydrolytically stable TCO— M^{M} conjugates are applied. The drawn fused ring products may or may not tautomerize to other more favorable tautomers.

Hereunder, some nonlimiting model combinations of TCO-M^{M} conjugates and tetrazine Activators illustrate the possibilities for cascade elimination induced model M^{M} release from the retro Diels-Alder adduct. The M^{M}, whether or not via a linker, is preferably attached to a carbon atom that is adjacent to the double bond in the TCO ring.

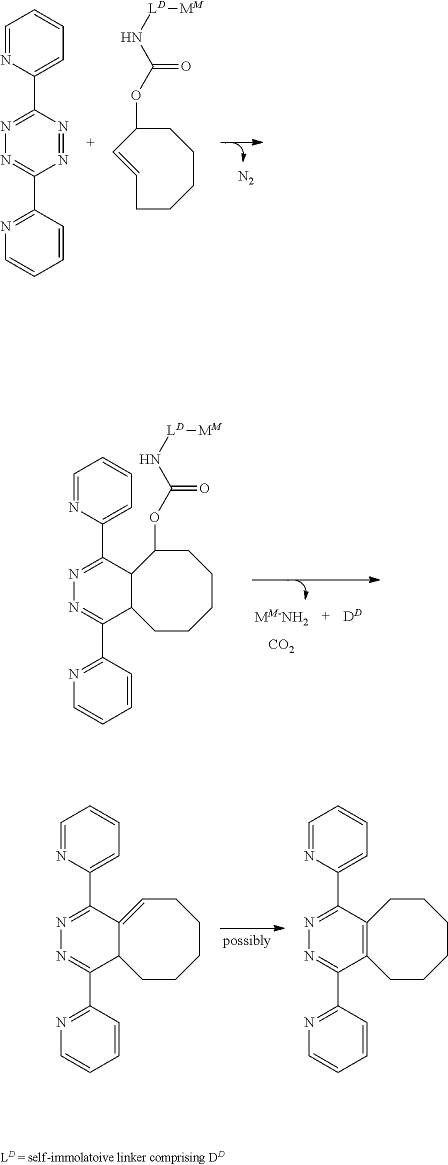

The above example of urethane (or carbamate) substituted TCOs gives release of an amine functional M^{M} from the adduct. The tetrazine Activator is symmetric and electron deficient.

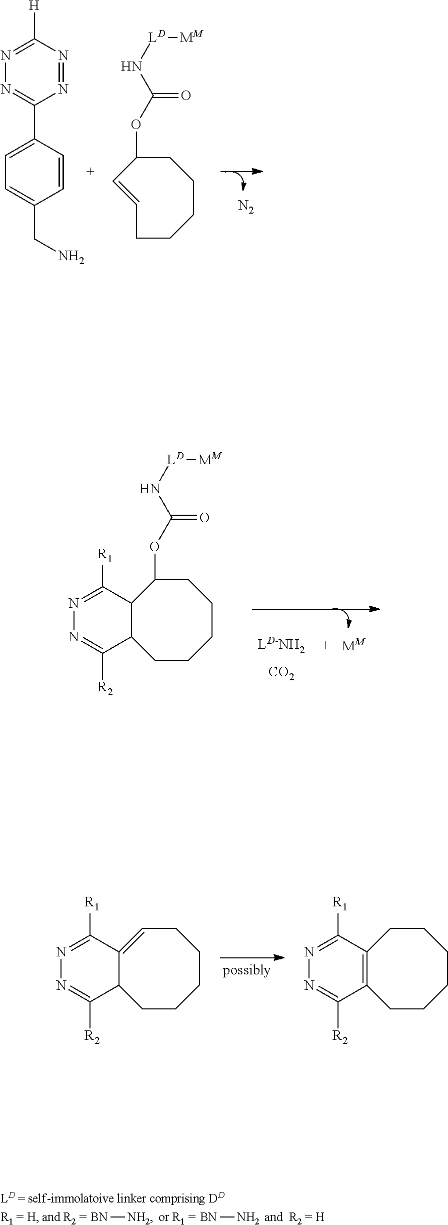

The above examples of urethane (or carbamate) substituted TCOs gives release of an amine functional M^{M} from the adduct. The tetrazine Activator is asymmetric and electron deficient. Note that use of an asymmetric tetrazine leads to formation of retro Diels-Alder adduct regiomers, apart from the stereo-isomers that are already formed when symmetric tetrazine are employed.

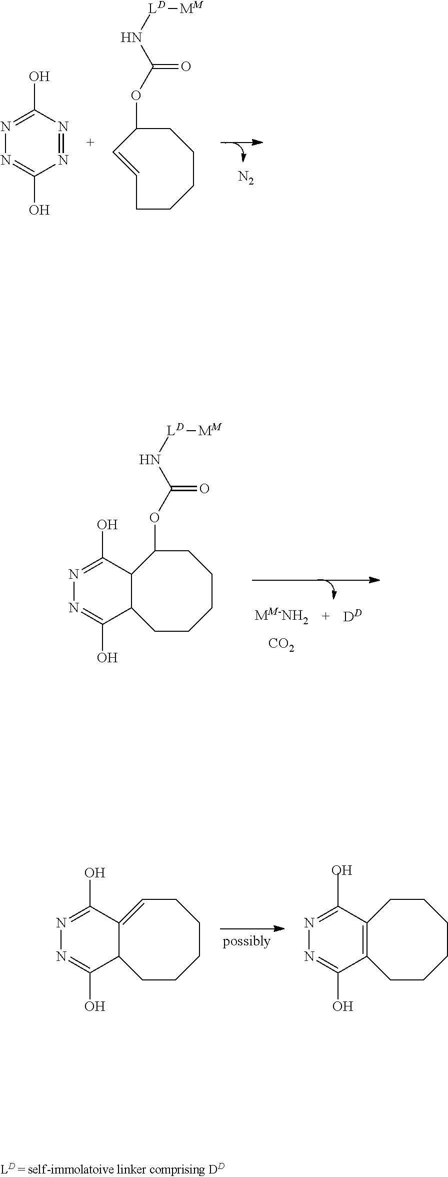

The above example of urethane (or carbamate) TCOs gives release of an amine functional M^{M} from the adduct. The tetrazine Activator is symmetric and electron sufficient.

The following schemes depict non-limiting examples illustrative for the various strain release mechanisms that can be made to apply on the basis of the choice for the rDA reaction for activating a Trigger-M^{M} conjugate.

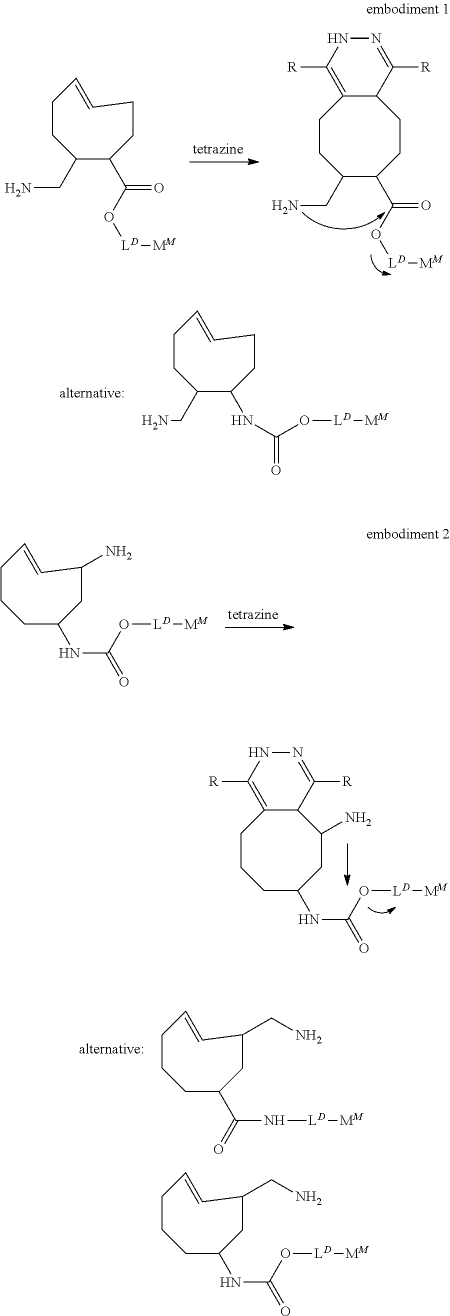

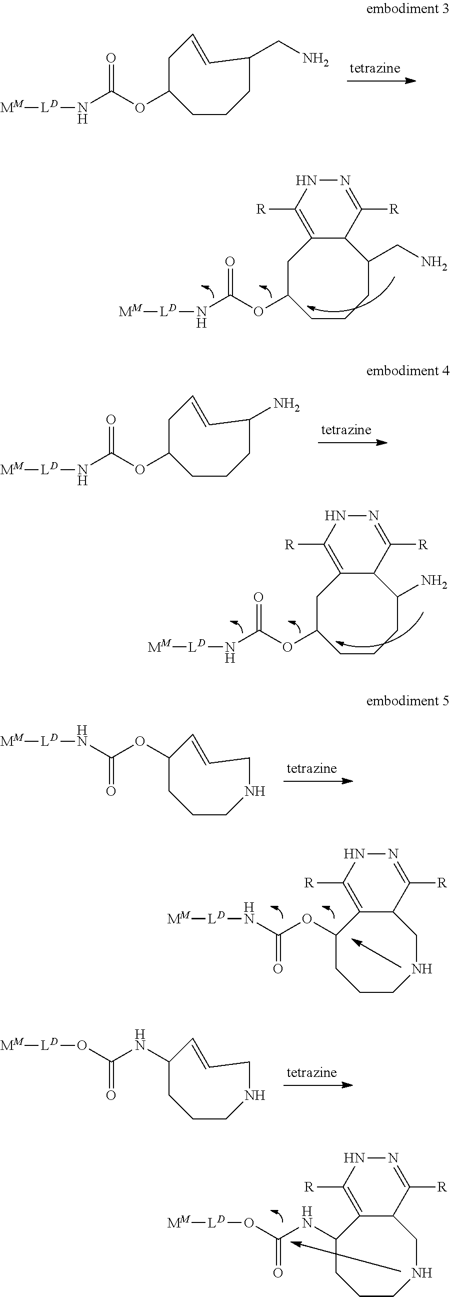

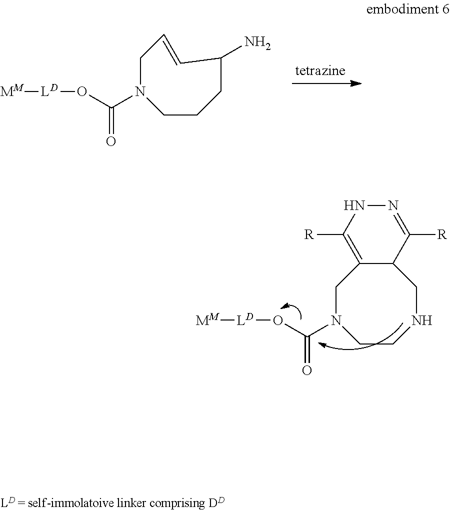

### The Activator

The Activator is a diene. The person skilled in the art is aware of the wealth of dienes that are reactive in the Retro Diels-Alder reaction. The diene comprised in the Activator can be part of a ring structure that comprises a third double bond, such as a tetrazine (which is a preferred Activator according to the invention).

Generally, the Activator is a molecule comprising a heterocyclic moiety comprising at least 2 conjugated double bonds.

Preferred dienes are given below, with reference to formulae (2)-(4).

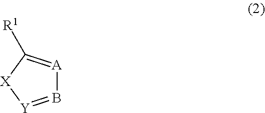

In formula (2) R¹ is selected from the group consisting of H, alkyl, aryl, CF₃, CF₂—R′, OR′, SR′, C(═O)R′, C(═S)R′, C(═O)O—R′, C(═O)S—R′, C(═S)O—R′, C(═S)S—R″, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, NR′C(═O)NR″R″, NR′C(═S)NR″R″ with each R′ and each R″ independently being H, aryl or alkyl; A and B each independently are selected from the group consisting of alkyl-substituted carbon, aryl substituted carbon, nitrogen, N⁺O⁻, N⁺R with R being alkyl, with the proviso that A and B are not both carbon; X is selected from the group consisting of O, N-alkyl, and C═O, and Y is CR with R being selected from the group consisting of H, alkyl, aryl, C(═O)OR′, C(═O)SR′, C(═S)OR′, C(═S)SR′, C(═O)NR′R″ with R′ and R″ each independently being H, aryl or alkyl.

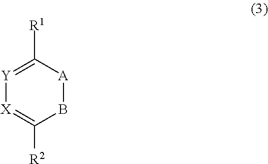

A diene particularly suitable as a reaction partner for cyclooctene is given in formula (3), wherein R¹ and R² each independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl; A is selected from the group consisting of N-alkyl, N-aryl, C═O, and CN-alkyl; B is O or S; X is selected from the group consisting of N, CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, CC(═S)NR′R″, R′ and R″ each independently being H, aryl or alkyl and R′″ independently being aryl or alkyl; Y is selected from the group consisting of CH, C-alkyl, C-aryl, N, and N+O—.

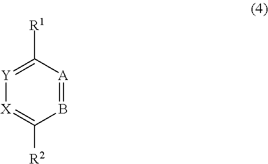

Another diene particularly suitable as a reaction partner for cyclooctene is diene (4), wherein Wand R² each independently are selected from the group consisting of H, alkyl, aryl, CF₃, CF₂—R′, NO, NO₂, OR′, SR′, CN, C(═O)R′, C(═S)R′, OC(═O)R′″, SC(═O)R′″, OC(═S)R′″, SC(═S)R′″, S(═O)R′, S(═O)₂R′″, S(═O)₂OR′, PO₃R′R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl; A is selected from the group consisting of N, C-alkyl, C-aryl, and N⁺O⁻; B is N; X is selected from the group consisting of N, CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, CC(═S)NR′R″, R′ and R″ each independently being H, aryl or alkyl and R′″ independently being aryl or alkyl; Y is selected from the group consisting of CH, C-alkyl, C-aryl, N, and N⁺O⁻.

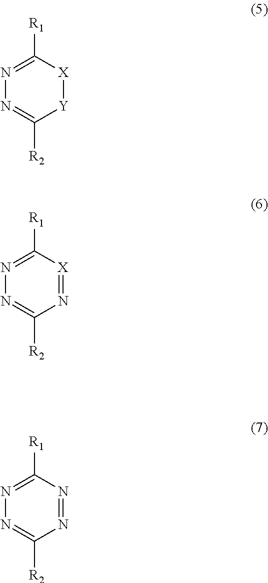

According to the invention, particularly useful dienes are 1,2-diazine, 1,2,4-triazine and 1,2,4,5-tetrazine derivatives, as given in formulas (5), (6) and (7), respectively.

The 1,2-diazine is given in (5), wherein R¹ and R² each independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl; X and Y each independently are selected from the group consisting of O, N-alkyl, N-aryl, C═O, CN-alkyl, CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, CC(═S)NR′R″, with R′ and R″ each independently being H, aryl or alkyl and R′″ independently being aryl or alkyl, where X—Y may be a single or a double bond, and where X and Y may be connected in a second ring structure apart from the 6-membered diazine. Preferably, X—Y represents an ester group (X=0 and Y═C═O; X—Y is a single bond) or X—Y represents a cycloalkane group (X═CR′ and Y═CR″; X—Y is a single bond; R′ and R″ are connected), preferably a cyclopropane ring, so that R′ and R″ are connected to each other at the first carbon atom outside the 1,2-diazine ring.

The 1,2,4-triazine is given in (6), wherein R¹ and R² each independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl; X is selected from the group consisting of CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, CC(═S)NR′R″, R′ and R″ each independently being H, aryl or alkyl and R′″ independently being aryl or alkyl.

The 1,2,4,5-tetrazine is given in (7), wherein R¹ and R² each independently are selected from the group consisting of H, alkyl, aryl, CF₃, CF₂—R′, NO, NO₂, OR′, SR′, CN, C(═O)R′, C(═S)R′, OC(═O)R′″, SC(═O)R′″, OC(═S)R′″, SC(═S)R′″, S(═O)R′, S(═O)₂R′″, S(═O)₂OR′, PO₃R′R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl.

Electron-deficient 1,2-diazines (5), 1,2,4-triazines (6) or 1,2,4,5-tetrazines (7) are especially interesting as such dienes are generally more reactive towards dienophiles. Di- tri- or tetra-azines are electron deficient when they are substituted with groups or moieties that do not generally hold as electron-donating, or with groups that are electron-withdrawing. For example, R¹ and/or R² may denote a substituent selected from the group consisting of H, alkyl, NO₂, F, CI, CF₃, CN, COOR, CONHR, CONR₂, COR, SO₂R, SO₂OR, SO₂NR₂, PO₃R₂, NO, 2-pyridyl, 3-pyridyl, 4-pyridyl, 2,6-pyrimidyl, 3,5-pyrimidyl, 2,4-pyrimidyl, 2,4 imidazyl, 2,5 imidazyl or phenyl, optionally substituted with one or more electron-withdrawing groups such as NO₂, F, CI, CF₃, CN, COOR, CONHR, CONR, COR, SO₂R, SO₂OR, SO₂NR₂, PO₃R₂, NO, Ar, wherein R is H or C₁-C₆ alkyl, and Ar stands for an aromatic group, particularly phenyl, pyridyl, or naphthyl.

The 1,2,4,5-tetrazines of formula (7) are most preferred as Activator dienes, as these molecules are most reactive in retro Diels-Alder reactions with dienophiles, such as the preferred TCO dienophiles, even when the R¹ and/or R² groups are not necessarily electron withdrawing, and even when R¹ and/or R² are in fact electron donating. Electron donating groups are for example OH, OR′, SH, SR′, NH₂, NHR′, NR′R″, NHC(═O)R″, NR′C(═O)R″, NHC(═S)R″, NR′C(═S)R″, NHSO₂R″, NR′SO₂R″ with R′ and R″ each independently being alkyl or aryl groups. Examples of other electron donating groups are phenyl groups with attached to them one or more of the electron donating groups as mentioned in the list above, especially when substituted in the 2-, 4- and/or 6-position(s) of the phenyl group.

According to the invention, 1,2,4,5-tetrazines with two electron withdrawing residues, or those with one electron withdrawing residue and one residue that is neither electron withdrawing nor donating, are called electron deficient. In a similar way, 1,2,4,5-tetrazines with two electron donating residues, or those with one electron donating residue and one residue that is neither electron withdrawing nor donating, are called electron sufficient. 1,2,4,5-Tetrazines with two residues that are both neither electron withdrawing nor donating, or those that have one electron withdrawing residue and one electron donating residue, are neither electron deficient nor electron sufficient.

The 1,2,4,5-tetrazines can be asymmetric or symmetric in nature, i.e. the R¹ and R² groups in formula (7) may be different groups or may be identical groups, respectively. Symmetric 1,2,4,5-tetrazines are more convenient as these Activators are more easily accessible via synthetic procedures.

We have tested several 1,2,4,5-tetrazines with respect to their ability as Activator to release a model M^{M} compound (e.g. benzyl amine) from a Prodrug, and we have found that tetrazines that are electron deficient, electron sufficient or neither electron deficient nor electron sufficient are capable to induce the M^{M} release. Furthermore, both symmetric as well as asymmetric tetrazines were effective.

Electron deficient 1,2,4,5 tetrazines and 1,2,4,5-tetrazines that are neither electron deficient nor electron sufficient are generally more reactive in retro Diels-Alder reactions with dienophiles (such as TCOs), so these two classes of 1,2,4,5-tetrazines are preferred over electron sufficient 1,2,4,5-tetrazines, even though the latter are also capable of inducing M^{M} release in Prodrugs.

In the following paragraphs specific examples of 1,2,4,5-tetrazine Activators according to the second embodiment of this invention will be highlighted by defining the R¹ and R² residues in formula (7).

Symmetric electron deficient 1,2,4,5-tetrazines with electron withdrawing residues are for example those with R¹═R²═H, 2-pyridyl, 3-pyridyl, 4-pyridyl, 2,4-pyrimidyl, 2,6-pyrimidyl, 3,5-pyrimidyl, 2,3,4-triazyl or 2,3,5-triazyl. Other examples are those with R¹═R²=phenyl with COOH or COOMe carboxylate, or with CN nitrile, or with CONH₂, CONHCH₃ or CON(CH₃)₂ amide, or with SO₃H or SO₃Na sulfonate, or with SO₂NH₂, SO₂NHCH₃ or SO₂N(CH₃)₂ sulfonamide, or with PO₃H₂ or PO₃Na₂ phosphonate substituents in the 2-, 3- or 4-position of the phenyl group, or in the 3- and 5-positions, or in the 2- and 4-positions, or in the 2,- and 6-positions of the phenyl group. Other substitution patterns are also possible, including the use of different substituents, as long as the tetrazine remains symmetric. See below for some examples of these structures.

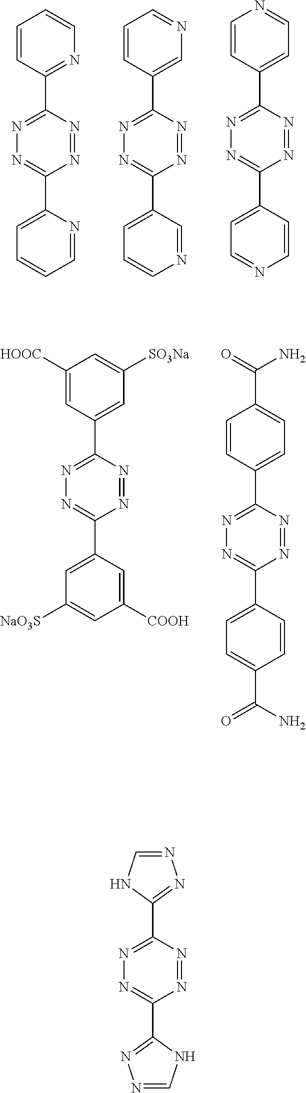

Symmetric electron sufficient 1,2,4,5-tetrazines with electron donating residues are for example those with R¹═R²═OH, OR′, SH, SR′, NH₂, NHR′, NR′₂, NH—CO—R′, NH—SO—R′, NH—SO₂—R′, 2-pyrryl, 3-pyrryl, 2-thiophene, 3-thiophene, where R′ represents a methyl, ethyl, phenyl or tolyl group. Other examples are those with R¹═R²=phenyl with OH, OR′, SH, SR′, NH₂, NHR′, NR′₂, NH—CO—R′, NR″—CO—R′, NH—SO—R′ or NH—SO₂—R′ substituent(s), where R′ represents a methyl, ethyl, phenyl or tolyl group, where R″ represents a methyl or ethyl group, and where the substitution is done on the 2- or 3- or 4- or 2- and 3- or 2- and 4- or 2- and 5- or 2- and 6- or 3- and 4- or 3- and 5- or 3-, 4- and 5-position(s). See below for some examples of these structures.

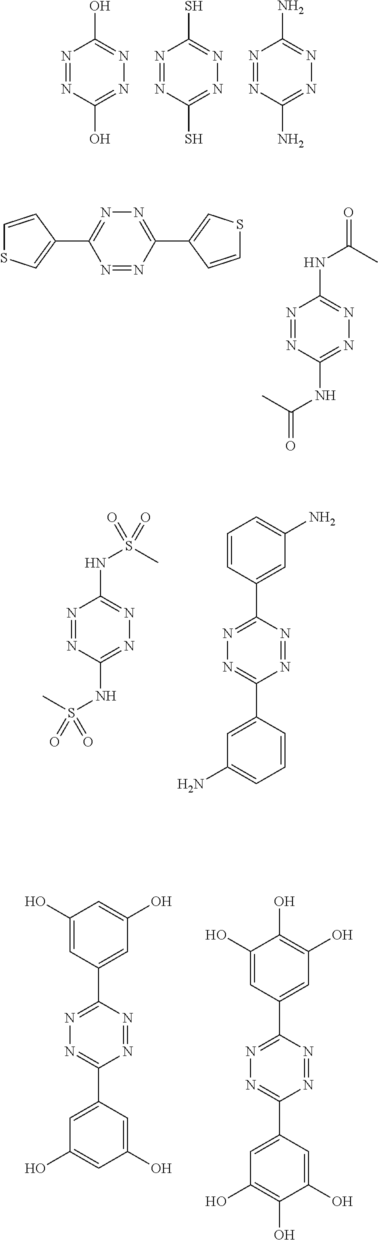

Symmetric 1,2,4,5-tetrazines with neither electron withdrawing nor electron donating residues are for example those with R¹═R²=phenyl, methyl, ethyl, (iso)propyl, 2,4-imidazyl, 2,5-imidazyl, 2,3-pyrazyl or 3,4-pyrazyl. Other examples are those where R¹═R²=a hetero(aromatic) cycle such as a oxazole, isoxazole, thiazole or oxazoline cycle. Other examples are those where R¹═R²=a phenyl with one electron withdrawing substituent selected from COOH, COOMe, CN, CONH₂, CONHCH₃, CON(CH₃)₂, SO₃H, SO₃Na, SO₂NH₂, SO₂NHCH₃, SO₂N(CH₃)₂, PO₃H₂ or PO₃Na₂ and one electron donating substituent selected from OH, OR′, SH, SR′, NH₂, NHR′, NR′₂, NH—CO—R′, NR″—CO—R′, NH—SO—R′ or NH—SO₂—R′ substituent(s), where R′ represents a methyl, ethyl, phenyl or tolyl group and where R″ represents a methyl or ethyl group. Substitutions can be done on the 2- and 3-, 2- and 4-, 2,- and 5-, 2- and 6, 3- and 4-, and the 3- and 5-positions. Yet other examples are those where R¹═R²=a pyridyl or pyrimidyl moiety with one electron donating substituent selected from OH, OR′, SH, SR′, NH₂, NHR′, NR′₂, NH—CO—R′, NR″—CO—R′, NH—SO—R′ or NH—SO₂—R′ substituents, where R′ represents a methyl, ethyl, phenyl or tolyl group and where R″ represents a methyl or ethyl group. See below for some examples.

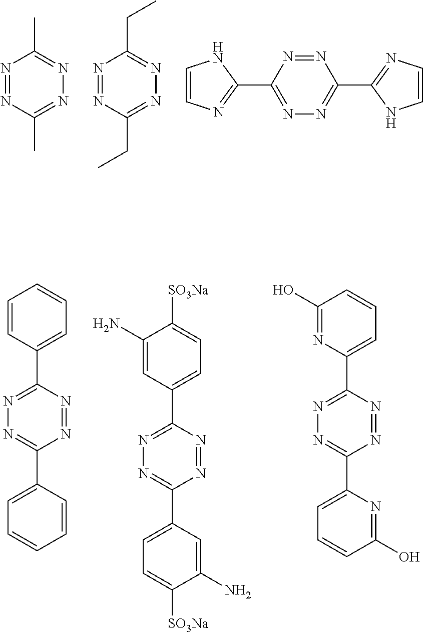

In case asymmetric 1,2,4,5-tetrazines are considered, one can choose any combination of given R¹ and R² residues that have been highlighted and listed above for the symmetric tetrazines according to formula (7), provided of course that R¹ and R² are different. Preferred asymmetric 1,2,4,5-tetrazines are those where at least one of the residues R¹ or R² is electron withdrawing in nature. Find below some example structures drawn.

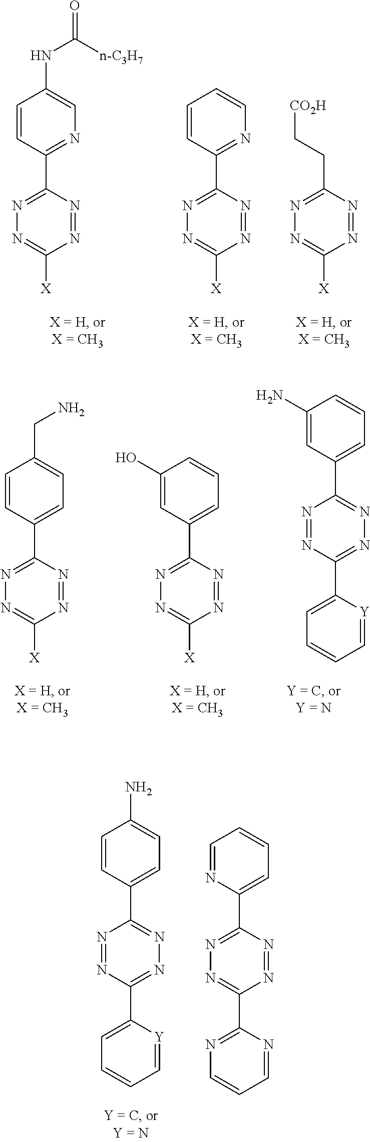

### Further Considerations Regarding the Activator

Preferred Activators are 1,2-diazines, 1,2,4-triazines and 1,2,4,5-tetrazines, particularly 1,2,4,5-tetrazines, are the preferred diene Activators. In the below, some relevant features of the Activator will be highlighted, where it will also become apparent that there are plentiful options for designing the right Activator formulation for every specific application.

According to the invention, the Activator, e.g. a 1,2,4,5-tetrazine, has useful and beneficial pharmacological and pharmaco-kinetic properties, implying that the Activator is non-toxic or at least sufficiently low in toxicity, produces metabolites that are also sufficiently low in toxicity, is sufficiently soluble in physiological solutions, can be applied in aqueous or other formulations that are routinely used in pharmaceutics, and has the right log D value where this value reflects the hydrophilic/hydrophobic balance of the Activator molecule at physiological pH. As is known in the art, log D values can be negative (hydrophilic molecules) or positive (hydrophobic molecules), where the lower or the higher the log D values become, the more hydrophilic or the more hydrophobic the molecules are, respectively. Log D values can be predicted fairly adequately for most molecules, and log D values of Activators can be tuned by adding or removing polar or apolar groups in their designs. Find below some Activator designs with their corresponding calculated log D values (at pH=7.4). Note that addition of methyl, cycloalkylene, pyridine, amine, alcohol or sulfonate groups or deletion of phenyl groups modifies the log D value, and that a very broad range of log D values is accessible.

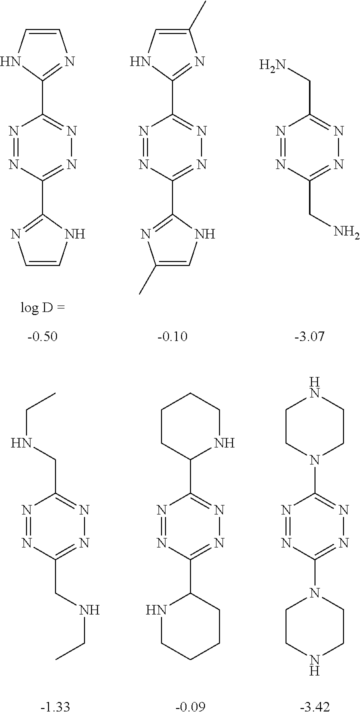

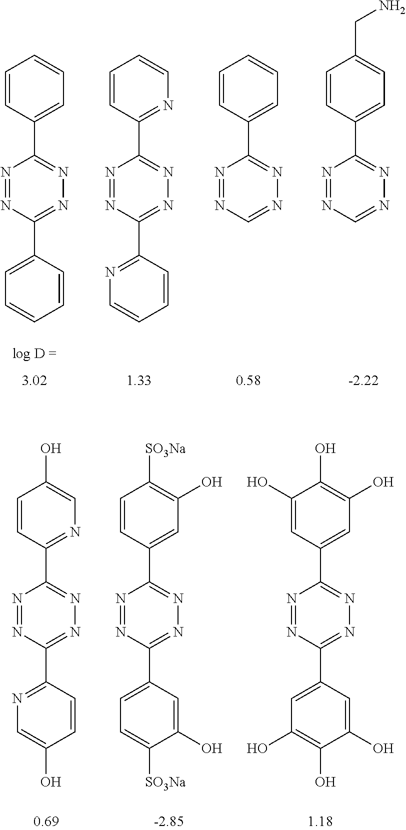

The given log D numbers have been calculated from a weighed method, with equal importance of the ‘VG’ (Viswanadhan, V. N.; Ghose, A. K.; Revankar, G. R.; Robins, R. K., J. Chem. Inf. Comput. Sci., 1989, 29, 163-172), ‘KLOP’ (according to Klopman, G.; Li, Ju-Yun.; Wang, S.; Dimayuga, M.: J. Chem. Inf. Comput. Sci., 1994, 34, 752) and ‘PHYS’ (according to the PHYSPROP© database) methods, based on an aqueous solution in 0.1 M in Na⁺/K⁺Cl⁻.

The Activator according to the invention has an appropriate reactivity towards the Prodrug, and this can be regulated by making the diene, particularly the 1,2,4,5-tetrazines, sufficiently electron deficient. Sufficient reactivity will ensure a fast retro Diels-Alder reaction with the Prodrug as soon as it has been reached by the Activator.

The Activator according to the invention has a good bio-availability, implying that it is available inside the (human) body for executing its intended purpose: effectively reaching the Prodrug at the Target. Accordingly, the Activator does not stick significantly to blood components or to tissue that is non-targeted. The Activator may be designed to bind to albumin proteins that are present in the blood (so as to increase the blood circulation time, as is known in the art), but it should at the same time be released effectively from the blood stream to be able to reach the Prodrug. Accordingly, blood binding and blood releasing should then be balanced adequately. The blood circulation time of the Activator can also be increased by increasing the molecular weight of the Activator, e.g. by attaching polyethylene glycol (PEG) groups to the Activator (‘pegylation’). Alternatively, the PKPD of the activator may be modulated by conjugating the activator to another moiety such as a polymer, protein, (short) peptide, carbohydrate.

The Activator according to the invention may be multimeric, so that multiple diene moieties may be attached to a molecular scaffold, particularly to e.g. multifunctional molecules, carbohydrates, polymers, dendrimers, proteins or peptides, where these scaffolds are preferably water soluble. Examples of scaffolds that can be used are (multifunctional) polyethylene glycols, poly (propylene imine) (PPI) dendrimers, PAMAM dendrimers, glycol based dendrimers, heparin derivatives, hyaluronic acid derivatives or serum albumine proteins such as HSA.

Depending on the position of the Prodrug (e.g. inside the cell or outside the cell; specific organ that is targeted) the Activator is designed to be able to effectively reach this Prodrug. Therefore, the Activator can for example be tailored by varying its log D value, its reactivity or its charge. The Activator may even be engineered with a targeting agent (e.g. a protein, a peptide and/or a sugar moiety), so that the Target can be reached actively instead of passively. In case a targeting agent is applied, it is preferred that it is a simple moiety (i.e. a short peptide or a simple sugar).

According to the invention, a mixture of different Activators can be applied. This may be relevant for regulation of the release profile of the drug.

The Activator that according to the invention will cause and regulate drug release at the Target may additionally be modified with moieties giving extra function(s) to the Activator, either for in-vitro and/or for in-vivo studies or applications. For example, the Activator may be modified with dye moieties or fluorescent moieties (see e.g. S. Hilderbrand et al., Bioconjugate Chem., 2008, 19, 2297-2299 for 3-(4-benzylamino)-1,2,4,5-tetrazine that is amidated with the near-infrared (NIR) fluorophore VT680), or they may be functionalized with imaging probes, where these probes may be useful in imaging modalities, such as the nuclear imaging techniques PET or SPECT. In this way, the Activator will not only initiate drug release, but can also be localized inside the (human) body, and can thus be used to localize the Prodrug inside the (human) body. Consequently, the position and amount of M^{M} release can be monitored. For example, the Activator can be modified with DOTA (or DTPA) ligands, where these ligands are ideally suited for complexation with “¹¹¹In³⁺-ions for nuclear imaging. In other examples, the Activator may be linked to ¹²³I or ¹⁸F moieties, that are well established for use in SPECT or PET imaging, respectively. Furthermore, when used in combination with e.g. beta-emitting isotopes, such as Lu-177, or Y-90, prodrug activation can be combined with localized radiotherapy in a pretargeted format.

Preferred activators are with Triggers based on the cascade mechanism:

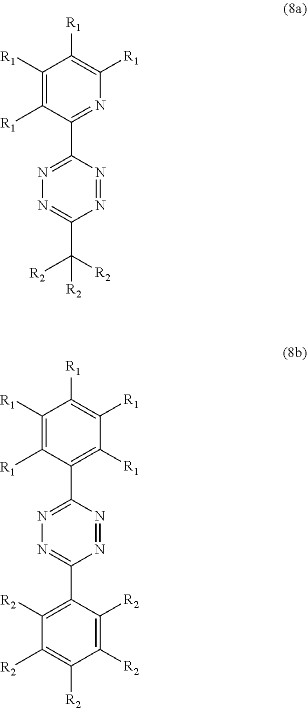

The 1,2,4,5-tetrazine given in Formula (8a) and (8b), wherein each R¹ and each R² independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl.

Other preferred activators for use with Triggers based on the cascade mechanism are:

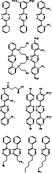

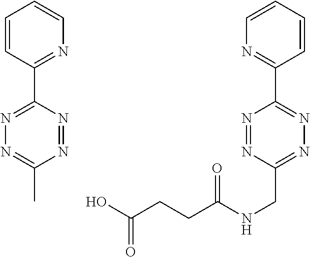 [SMILES: Cc1nnc(-c2ccccn2)nn1 ; O=C(O)CCC(=O)NCc1nnc(-c2ccccn2)nn1]

Preferred activators for use with Triggers based on the strain release mechanism are

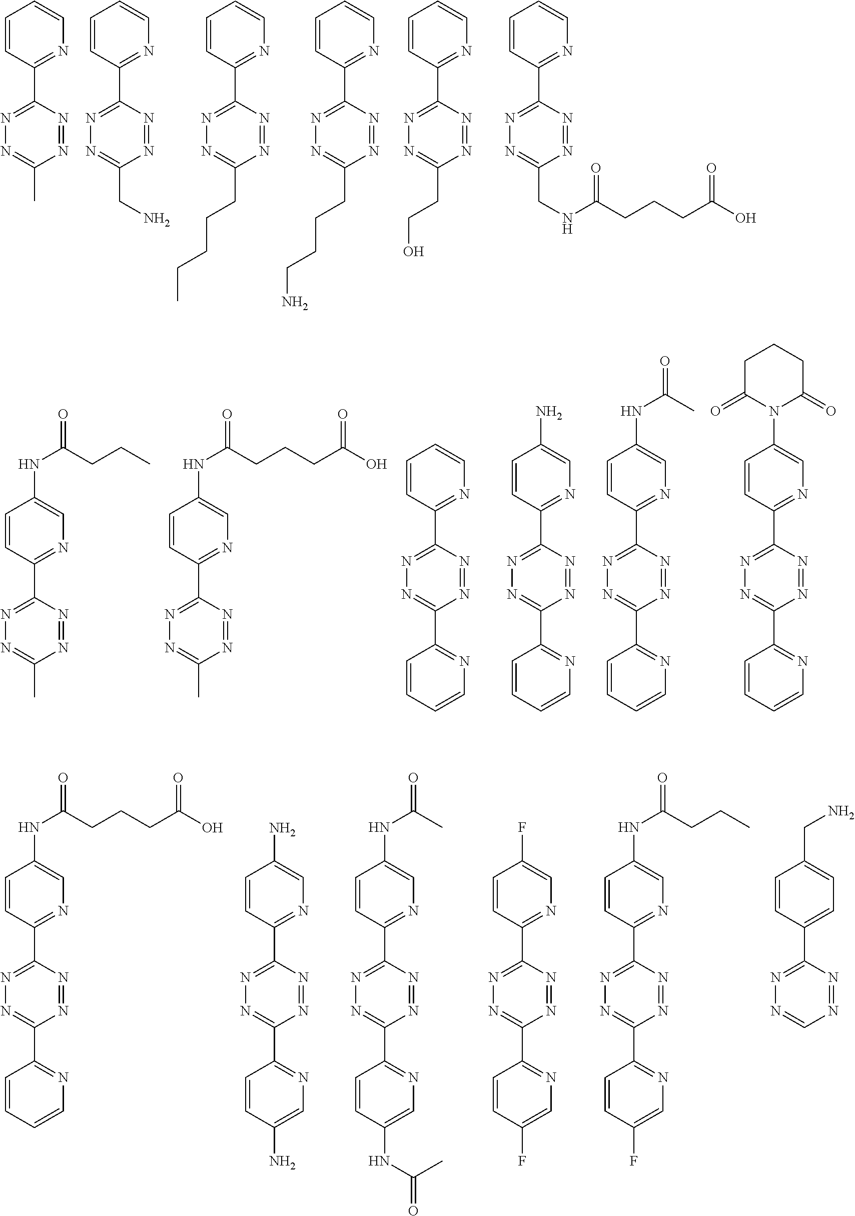

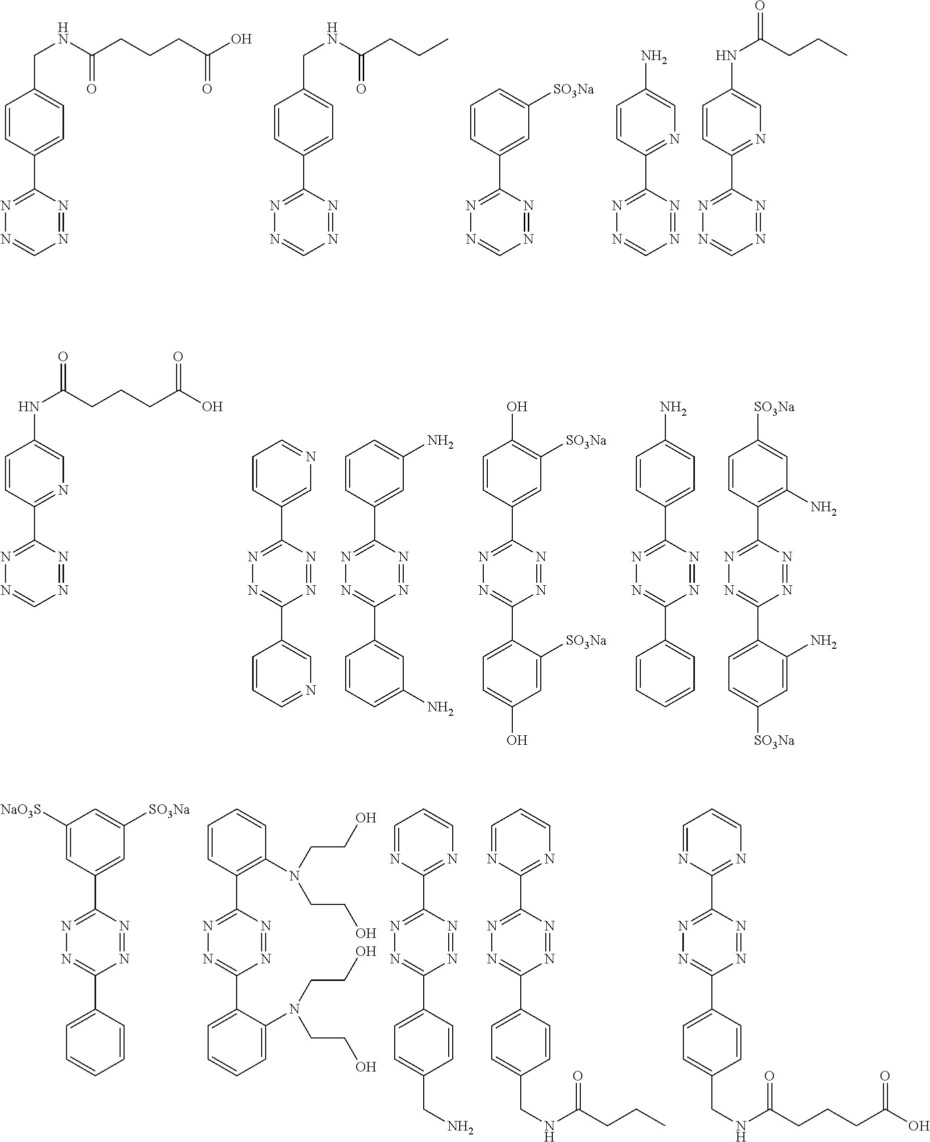

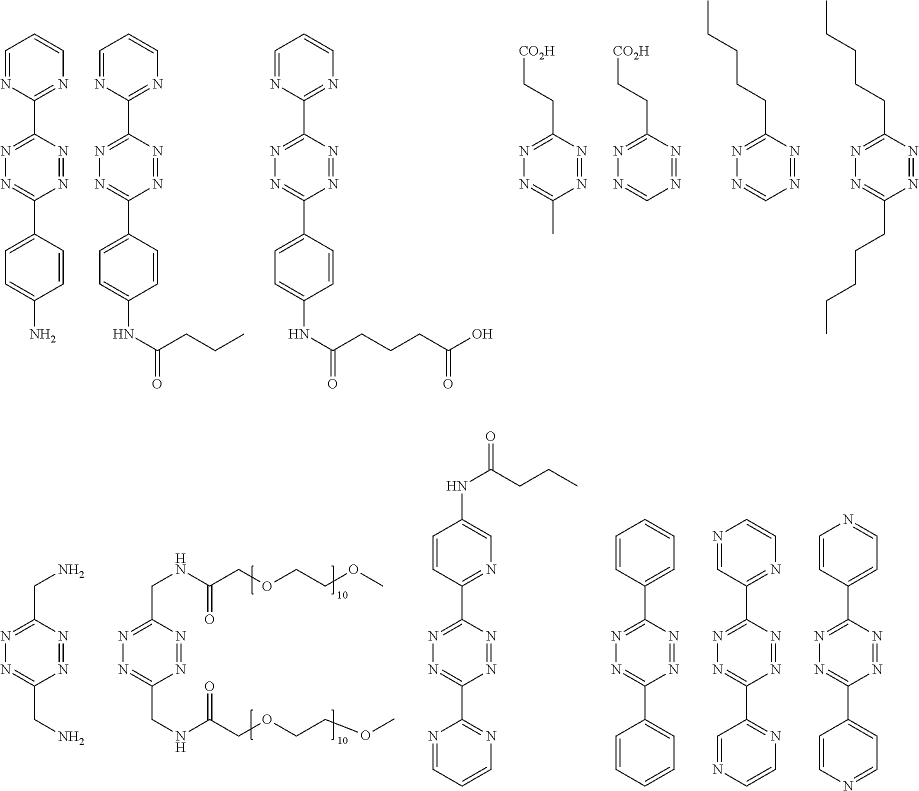

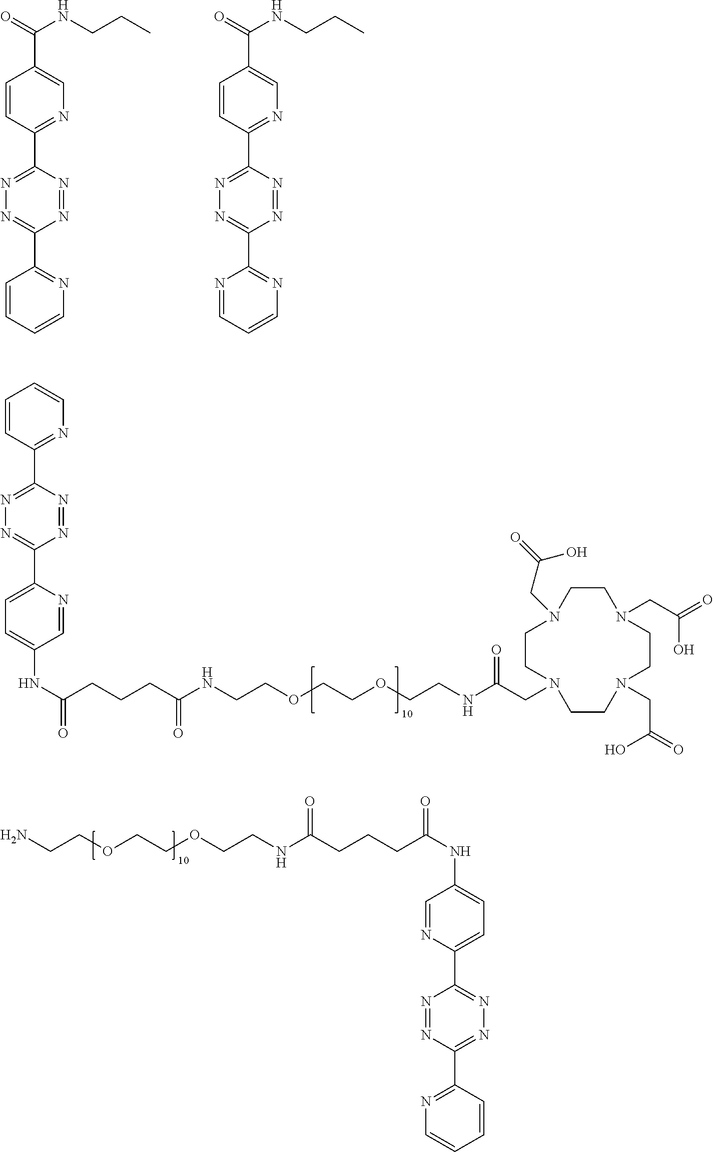

The Activator can have a link to a Masking Moiety M^{M} such as a peptide, protein, carbohydrate, PEG, or polymer. Preferably, these Activators for use with Triggers based on the cascade mechanism satisfy one of the following formulae:

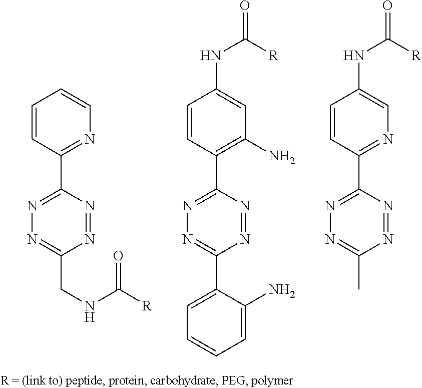

Preferably, these Activators for use with Triggers based on the strain release mechanism, satisfy one of the following formulae:

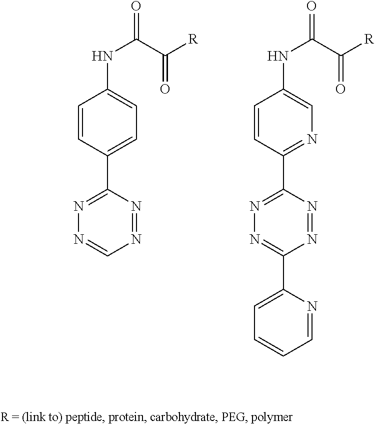

Synthesis routes to the above activators are readily available to the skilled person, based on standard knowledge in the art. References to tetrazine synthesis routes include Lions et al, J. Org. Chem., 1965, 30, 318-319; Horwitz et al, J. Am. Chem. Soc., 1958, 80, 3155-3159; Hapiot et al, New. J. Chem., 2004, 28, 387-392, Kaim et al, Z. Naturforsch., 1995, 50b, 123-127.

### Prodrug

A Prodrug is a conjugate of the Drug D^{D} and the Trigger T^{R} and M^{M}, and thus comprises a Drug that is capable of therapeutic action after its release from of the M^{M}. Such a Prodrug may optionally have specificity for disease targets.

The general formula of the Prodrug is shown below in Formula (9a) and (9b).

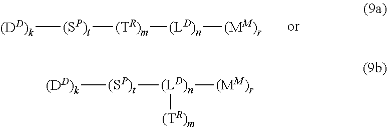

M^{M} is masking moiety; S^{P} is spacer; T^{R} is Trigger, L^{D} is linker, and D^{D} is drug. Formula (9a): k=1; m,r≧1; t,n≧0. Formula (9b): k=1; m,n,r≧1; t≧0.

Although it has been omitted for the sake of clarity in the above formula, D^{D} can further comprise T^{T}, optionally via S^{P}.

It will be understood that formula 1a and 1 b describe the Trigger and describe how the Trigger is attached to D^{D}, L^{D}, S^{P}, M^{M}, but that species D^{D}, L^{D}, S^{P}, M^{M} are not part of the Trigger and should be viewed as separate entities, as can be seen in e.g. Scheme 1 and formula 9.

Drugs that can be used in a Prodrug relevant to this invention include but are not limited to: antibodies, antibody derivatives, antibody fragments, e.g. Fab2, Fab, scFV, diabodies, triabodies, antibody (fragment) fusions (eg bi-specific and trispecific mAb fragments), proteins, aptamers, oligopeptides, oligonucleotides, oligosaccharides, as well as peptides, peptoids, toxins, hormones, viruses, whole cells, phage. Typical drugs for which the invention is suitable include, but are not limited to: bi-specific and trispecific mAb fragments, immunotoxins, comprising eg ricin A, diphtheria toxin, cholera toxin. Other embodiments use antiproliferative/antitumor agents, antibiotics, cytokines, anti-inflammatory agents, anti-viral agents, antihypertensive agents, chemosensitizing and radiosensitizing agents. Drugs optionally include a membrane translocation moiety (adamantine, poly-lysine/argine, TAT) and/or a targeting agent (against eg a tumor cel receptor) optionally linked through a stable or labile linker.

Exemplary drugs for use as conjugates to the TCO derivative and to be released upon retro Diels Alder reaction with the Activator include but are not limited to: cytotoxic drugs, particularly those which are used for cancer therapy. Such drugs include, in general, DNA damaging agents, anti-metabolites, natural products and their analogs.

It is preferred that the drug is a protein, antibody, antibody derivative or antibody fragment.

It will be understood that the Drug can optionally be attached to the TCO derivative through a spacer S^{P}. It will be understood that the invention encompasses any conceivable manner in which the dienophile Trigger is attached to the Drug. Methods of affecting conjugation to these drugs, e.g. through reactive amino acids such as lysine or cysteine in the case of proteins, are known to the skilled person. Protein conjugation techniques that retain the protein function are known in the art, such as in the antibody drug conjugate field, where e.g. use is made of engineered cysteines. Also reference is made to US20100189651.

It will further be understood that one ore more targeting agents T^{T} may optionally be attached to the Drug D^{D}, Trigger T^{R}, or Linker L^{D}, optionally via a spacer or spacers S^{P}.

According to a further particular embodiment of the invention, the Prodrug is selected so as to target and or address a disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme.

According to one embodiment, the Prodrug and/or the Activator can be multimeric compounds, comprising a plurality of Drugs and/or bioorthogonal reactive moieties. These multimeric compounds can be polymers, dendrimers, liposomes, polymer particles, or other polymeric constructs.

### Targeting

The kits and method of the invention are very suitable for use in targeted delivery of drugs.

A “primary target” as used in the present invention relates to a target for a targeting agent for therapy. For example, a primary target can be any molecule, which is present in an organism, tissue or cell. Targets include cell surface targets, e.g. receptors, glycoproteins; structural proteins, e.g. amyloid plaques; abundant extracullular targets such as stroma, extracellular matrix targets such as growth factors, and proteases; intracellular targets, e.g. surfaces of Golgi bodies, surfaces of mitochondria, RNA, DNA, enzymes, components of cell signaling pathways; and/or foreign bodies, e.g. pathogens such as viruses, bacteria, fungi, yeast or parts thereof. Examples of primary targets include compounds such as proteins of which the presence or expression level is correlated with a certain tissue or cell type or of which the expression level is up regulated or down-regulated in a certain disorder. According to a particular embodiment of the present invention, the primary target is a protein such as a (internalizing or non-internalizing) receptor.

According to the present invention, the primary target can be selected from any suitable targets within the human or animal body or on a pathogen or parasite, e.g. a group comprising cells such as cell membranes and cell walls, receptors such as cell membrane receptors, intracellular structures such as Golgi bodies or mitochondria, enzymes, receptors, DNA, RNA, viruses or viral particles, antibodies, proteins, carbohydrates, monosacharides, polysaccharides, cytokines, hormones, steroids, somatostatin receptor, monoamine oxidase, muscarinic receptors, myocardial sympatic nerve system, leukotriene receptors, e.g. on leukocytes, urokinase plasminogen activator receptor (uPAR), folate receptor, apoptosis marker, (anti-)angiogenesis marker, gastrin receptor, dopaminergic system, serotonergic system, GABAergic system, adrenergic system, cholinergic system, opoid receptors, GPIIb/IIIa receptor and other thrombus related receptors, fibrin, calcitonin receptor, tuftsin receptor, integrin receptor, fibronectin, VEGF/EGF and VEGF/EGF receptors, TAG72, CEA, CD19, CD20, CD22, CD40, CD45, CD74, CD79, CD105, CD138, CD174, CD227, CD326, CD340, MUC1, MUC16, GPNMB, PSMA, Cripto, Tenascin C, Melanocortin-1 receptor, CD44v6, G250, HLA DR, ED-B, TMEFF2, EphB2, EphA2, FAP, Mesothelin, GD2, CAIX, 5T4, matrix metalloproteinase (M^{M} P), P/E/L-selectin receptor, LDL receptor, P-glycoprotein, neurotensin receptors, neuropeptide receptors, substance P receptors, NK receptor, CCK receptors, sigma receptors, interleukin receptors, herpes simplex virus tyrosine kinase, human tyrosine kinase. In order to allow specific targeting of the above-listed primary targets, the targeting agent T^{T} can comprise compounds including but not limited to antibodies, antibody fragments, e.g. Fab2, Fab, scFV, diabodies, triabodies, VHH, antibody (fragment) fusions (eg bi-specific and trispecific mAb fragments), proteins, peptides, e.g. octreotide and derivatives, VIP, MSH, LHRH, chemotactic peptides, bombesin, elastin, peptide mimetics, carbohydrates, monosacharides, polysaccharides, viruses, whole cells, drugs, polymers, liposomes, chemotherapeutic agents, receptor agonists and antagonists, cytokines, hormones, steroids. Examples of organic compounds envisaged within the context of the present invention are, or are derived from, estrogens, e.g. estradiol, androgens, progestins, corticosteroids, methotrexate, folic acid, and cholesterol. In a preferred embodiment, the targeting agent T^{T} is an antibody. According to a particular embodiment of the present invention, the primary target is a receptor and a targeting agent is employed, which is capable of specific binding to the primary target. Suitable targeting agents include but are not limited to, the ligand of such a receptor or a part thereof which still binds to the receptor, e.g. a receptor binding peptide in the case of receptor binding protein ligands. Other examples of targeting agents of protein nature include interferons, e.g. alpha, beta, and gamma interferon, interleukins, and protein growth factor, such as tumor growth factor, e.g. alpha, beta tumor growth factor, platelet-derived growth factor (PDGF), uPAR targeting protein, apolipoprotein, LDL, annexin V, endostatin, and angiostatin. Alternative examples of targeting agents include DNA, RNA, PNA and LNA which are e.g. complementary to the primary target.

According to a further particular embodiment of the invention, the primary target and targeting agent are selected so as to result in the specific or increased targeting of a tissue or disease, such as cancer, an inflammation, an infection, a cardiovascular disease, e.g. thrombus, atherosclerotic lesion, hypoxic site, e.g. stroke, tumor, cardiovascular disorder, brain disorder, apoptosis, angiogenesis, an organ, and reporter gene/enzyme. This can be achieved by selecting primary targets with tissue-, cell- or disease-specific expression. For example, membrane folic acid receptors mediate intracellular accumulation of folate and its analogs, such as methotrexate. Expression is limited in normal tissues, but receptors are overexpressed in various tumor cell types.

### Masking Moieties

Masking moieties M^{M} can be a protein, peptide, polymer, polyethylene glycol, carbohydrate, organic construct, or a combination thereof that further shield the bound drug D^{D} or Prodrug. This shielding can be based on eg steric hindrance, but it can also be based on a non covalent interaction with the drug D^{D}. Such masking moiety may also be used to affect the in vivo properties (eg blood clearance; recognition by the immunesystem) of the drug D^{D} or Prodrug.

The M^{M} of the modified D^{D} can reduce the D^{D} 's ability to bind its target allosterically or sterically. In specific embodiments, the M^{M} is a peptide and does not comprise more than 50% amino acid sequence similarity to a natural binding partner of the antibody-based D^{D}.

In one embodiment the M^{M} reduces the ability of the D^{D} to bind its target such that that the dissociation constant of the D^{D} when coupled to the M^{M} towards the target is at least 100 times greater than the dissociation constant towards the target of the D^{D} when not coupled to the M^{M}.

In another embodiment, the coupling of the M^{M} to the D^{D} reduces the ability of the D^{D} to bind its target by at least 90%.

In the Prodrug, the M^{M} and the Trigger T^{R}—the TCO derivative—can be directly linked to each other. They can also be bound to each other via a linker or a self-immolative linker L^{D}. It will be understood that the invention encompasses any conceivable manner in which the dienophile Trigger is attached to the M^{M}. It will be understood that M^{M} is linked to the TCO in such a way that the M^{M} is eventually capable of being released after formation of the retro Diels-Alder adduct. Generally, this means that the bond between the drug and the TCO, or in the event of a linker, the bond between the TCO and the linker L^{D}, or in the event of a self-immolative linker L^{D}, the bond between the linker and the TCO and between the M^{M} and the linker, should be cleavable. Predominantly, the M^{M} and the optional linker is linked via a hetero-atom, preferably via O, N, NH, or S. The cleavable bond is preferably selected from the group consisting of carbamate, thiocarbamate, carbonate, ether, ester, amine, amide, thioether, thioester, sulfoxide, and sulfonamide bonds.

### Spacers

Spacers S^{P} include but are not limited to polyethylene glycol (PEG) chains varying from 2 to 200, particularly 3 to 113 and preferably 5-50 repeating units. Other examples are biopolymer fragments, such as oligo- or polypeptides or polylactides.

### Administration

In the context of the invention, the Prodrug is usually administered first, and it will take a certain time period before the Prodrug has reached the Primary Target. This time period may differ from one application to the other and may be minutes, days or weeks. After the time period of choice has elapsed, the Activator is administered, will find and react with the Prodrug and will thus activate M^{M} release at the Primary Target.

The compositions of the invention can be administered via different routes including intravenous injection, intraperatonial, oral administration, rectal administration and inhalation. Formulations suitable for these different types of administrations are known to the skilled person. Prodrugs or Activators according to the invention can be administered together with a pharmaceutically acceptable carrier. A suitable pharmaceutical carrier as used herein relates to a carrier suitable for medical or veterinary purposes, not being toxic or otherwise unacceptable. Such carriers are well known in the art and include saline, buffered saline, dextrose, water, glycerol, ethanol, and combinations thereof. The formulation should suit the mode of administration.

It will be understood that the chemical entities administered, viz. the prodrug and the activator, can be in a modified form that does not alter the chemical functionality of said chemical entity, such as salts, hydrates, or solvates thereof.

After administration of the Prodrug, and before the administration of the Activator, it is preferred to remove excess Prodrug by means of a Clearing Agent in cases when prodrug activation in circulation is undesired and when natural prodrug clearance is insufficient. A Clearing Agent is an agent, compound, or moiety that is administered to a subject for the purpose of binding to, or complexing with, an administered agent (in this case the Prodrug) of which excess is to be removed from circulation. The Clearing Agent is capable of being directed to removal from circulation. The latter is generally achieved through liver receptor-based mechanisms, although other ways of secretion from circulation exist, as are known to the skilled person. In the invention, the Clearing Agent for removing circulating Prodrug, preferably comprises a diene moiety, e.g. as discussed above, capable of reacting to the TCO moiety of the Prodrug.

### Additional Embodiment 1

With reference to formula (1a) and (1b) for Triggers that function via cascade-mediated release or elimination (i.e. cascade mechanism), when p=1 and n=1 it is preferred that L^{D} is linked to T^{R} via N or NH or an aliphatic or aromatic carbon, wherein these atoms are part of the linker; and when p=1 and n=0 it is preferred that M^{M} is linked to T^{R} via N or NH or an aliphatic or aromatic carbon, wherein these atoms are part of D^{D}. It is further preferred that said N and NH moieties comprised in L^{D} or M^{M} are bound to an aliphatic or aromatic carbon of L^{D} or M^{M}.

With reference to formula (1a) and (1b) for Triggers that function via cascade-mediated release or elimination (i.e. cascade mechanism), when p=0 and n=1 it is preferred that L^{D} is linked to T^{R} via S or O, wherein these atoms are part of the linker; and when p=0 and n=0 it is preferred that M^{M} is linked to T^{R} via S or O, wherein these atoms are part of M^{M}. It is further preferred that said S and O moieties comprised in L^{D} or M^{M} are bound to an aliphatic or aromatic carbon or carbonyl or thiocarbonyl of L^{D} or M^{M}.

With reference to formula (1a) and (1b) for Triggers that function via cascade-mediated release or elimination (i.e. cascade mechanism), in particular embodiments when X^{D} is S—C(O)-(L^{D})ₙ-(M^{M}), O—C(S)-(L^{D})ₙ-(M^{M}), S—C(S)-(L^{D})ₙ-(M^{M}) and n=1 it is preferred that L^{D} is linked to T^{R} via N or NH or an aliphatic or aromatic carbon, wherein these atoms are part of the linker; and when n=0 it is preferred that M^{M} is linked to T^{R} via N or NH or an aliphatic or aromatic carbon, wherein these atoms are part of M^{M}. It is further preferred that said N and NH moieties comprised in L^{D} or M^{M} are bound to an aliphatic or aromatic carbon of L^{D} or M^{M}.

### Additional Embodiment 2

Further preferred activators for use with Triggers based on the cascade mechanism are:

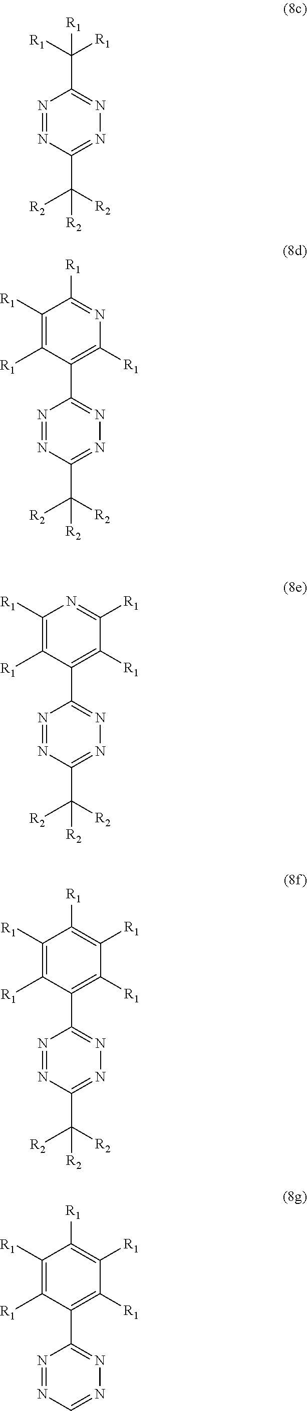

The 1,2,4,5-tetrazine given in Formula (8c-g), wherein each R¹ and each R² independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, NR′C(═S)NR″R″ with each R′ and each R″ independently being H, aryl or alkyl, and R′″ independently being aryl or alkyl.

Other preferred activators for use with Triggers based on the cascade mechanism are:

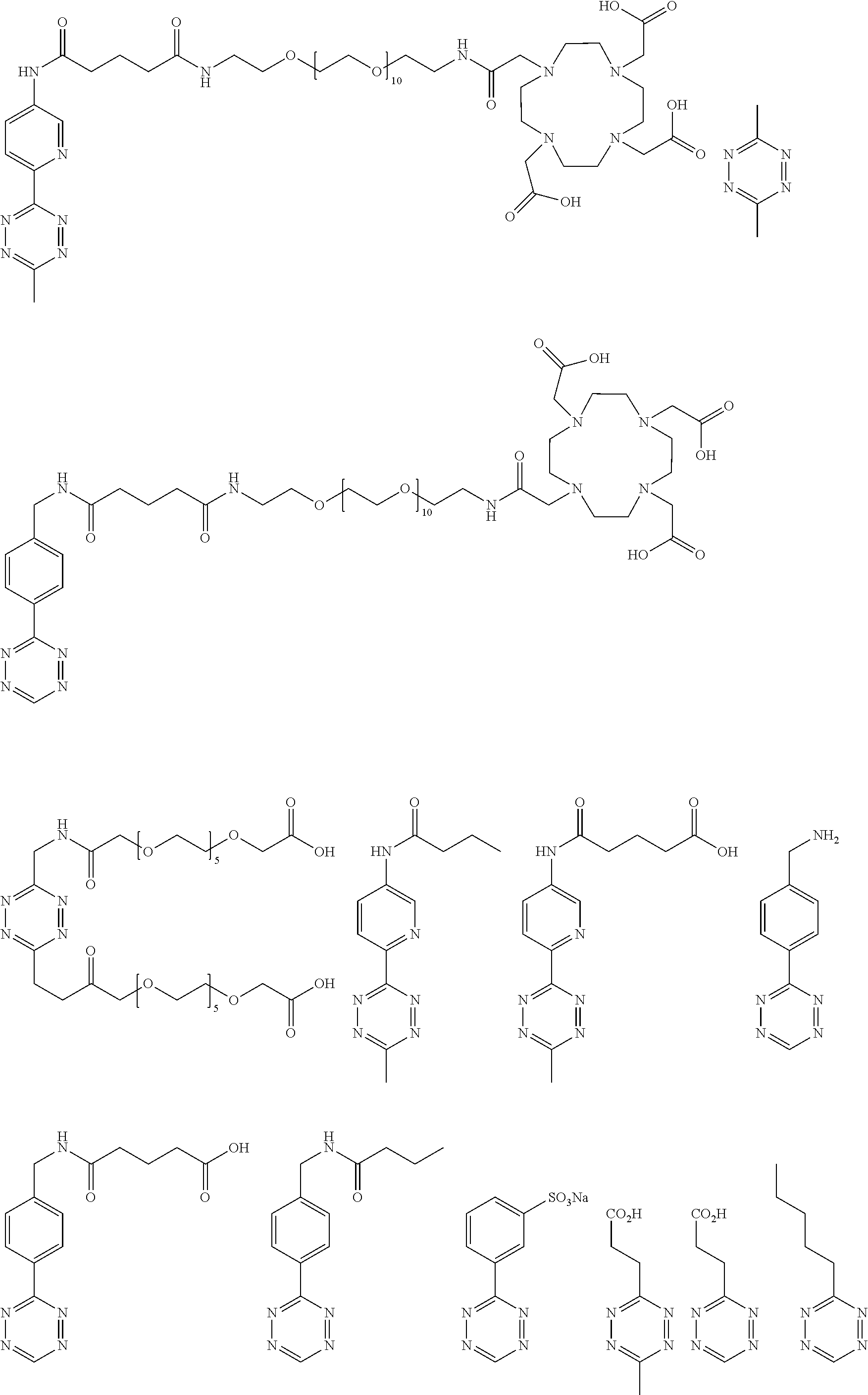

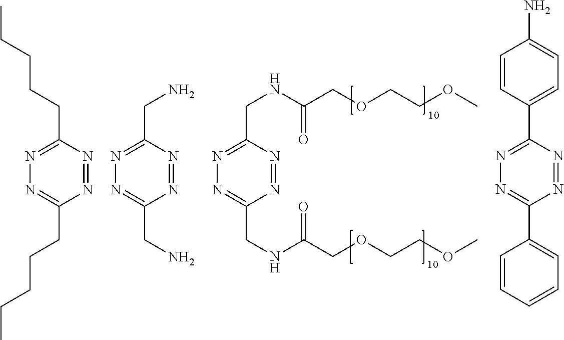

Other preferred activators for use with Triggers based on the strain release mechanism are:

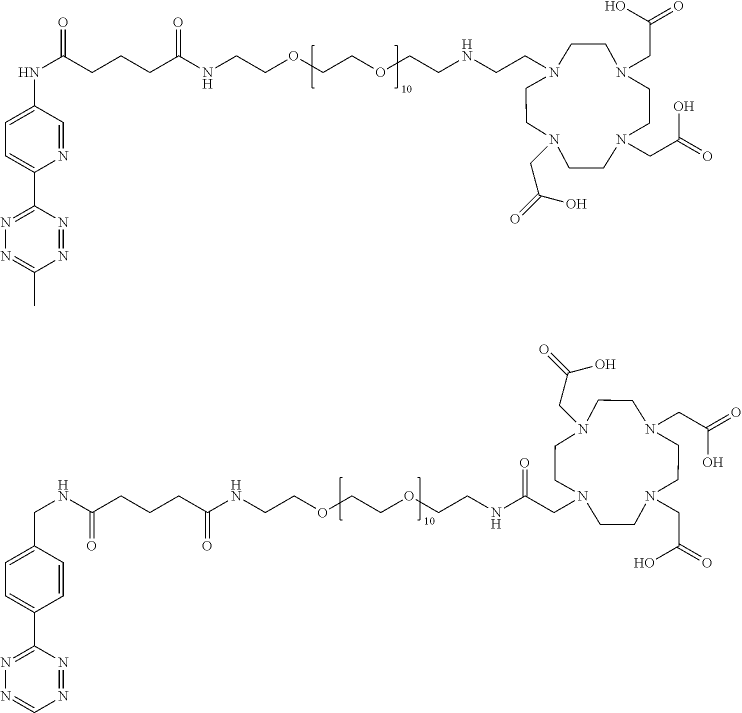

The Activator can have a link to a Masking Moiety M^{M} such as a peptide, protein, carbohydrate, PEG, or polymer. Preferably, these Activators for use with Triggers based on the cascade mechanism satisfy one of the following formulae:

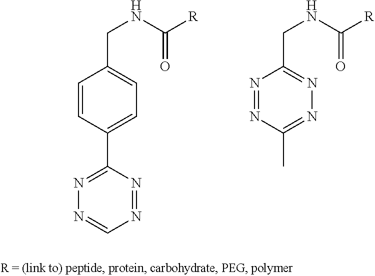

### Additional Embodiment 3

Some embodiments satisfy the one of the following formulas:

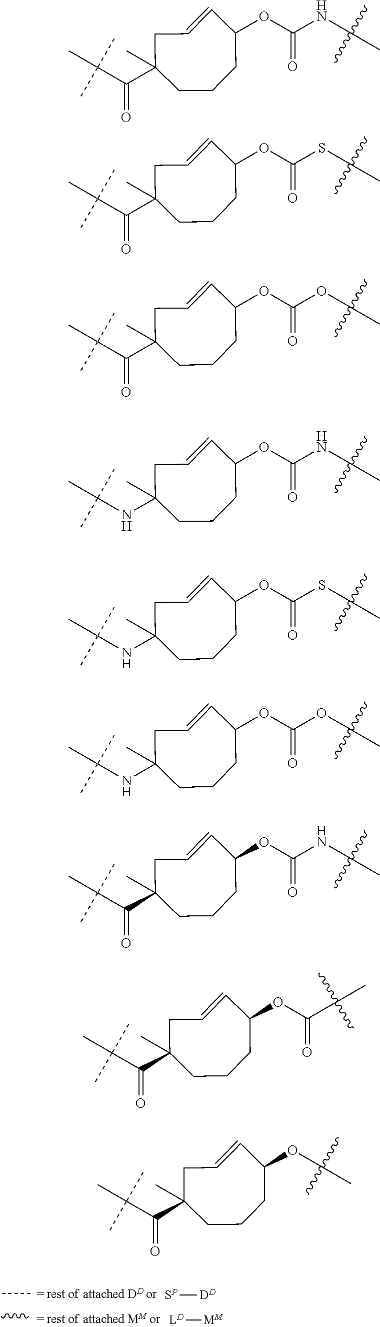

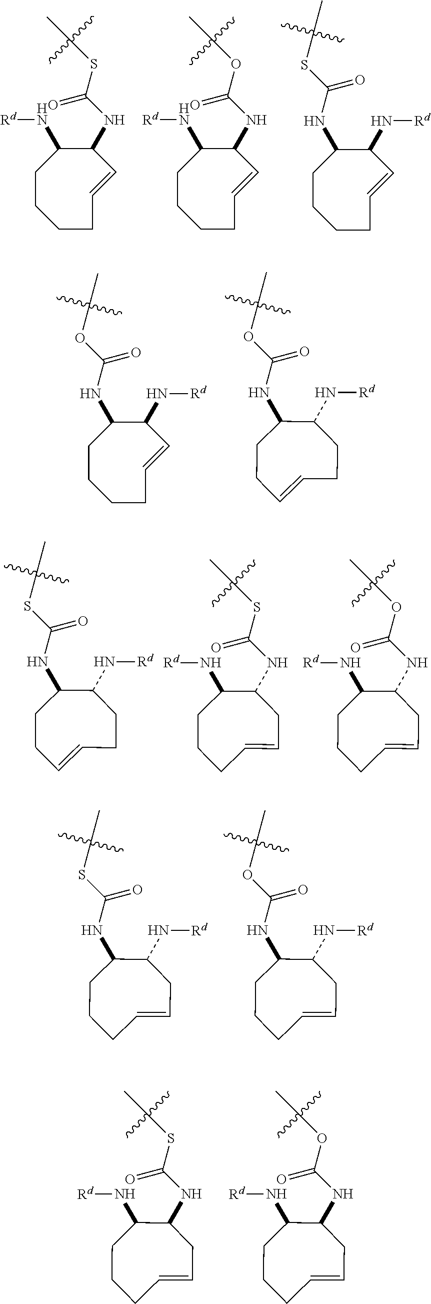

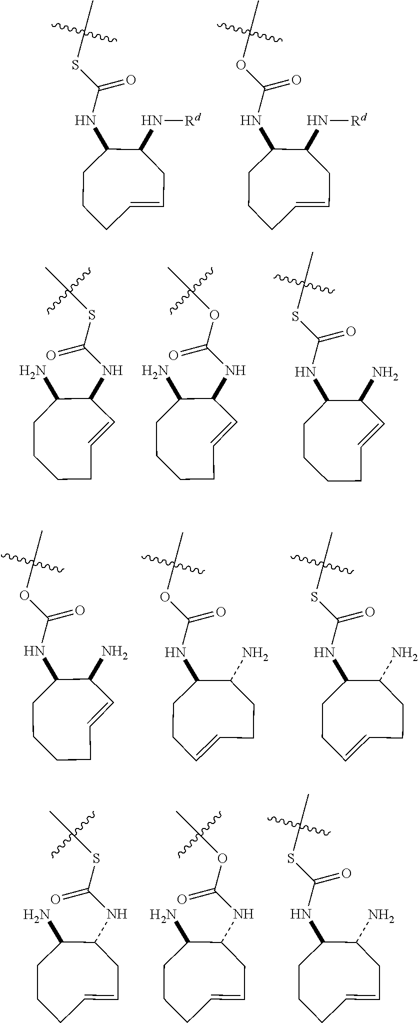

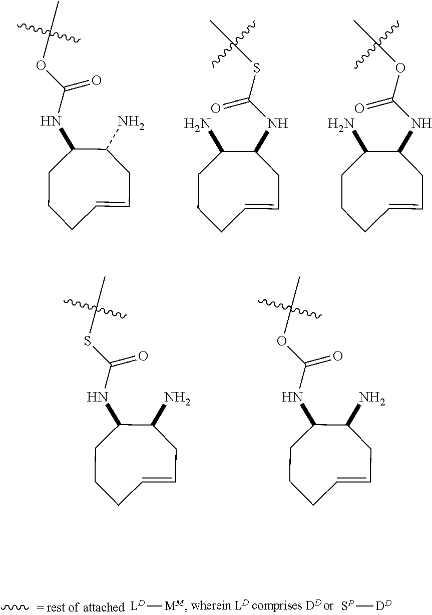

### EXAMPLES

The following examples demonstrate the invention or aspects of the invention, and do not serve to define or limit the scope of the invention or its claims.

Methods.

¹H-NMR and ¹³C-NMR spectra were recorded on a Varian Mercury (400 MHz for ¹H-NMR and 100 MHz for ¹³C-NMR) spectrometer at 298 K. Chemical shifts are reported in ppm downfield from TMS at room temperature. Abbreviations used for splitting patterns are s=singlet, t=triplet, q=quartet, m=multiplet and br=broad. IR spectra were recorded on a Perkin Elmer 1600 FT-IR (UATR). LC-MS was performed using a Shimadzu LC-10 AD VP series HPLC coupled to a diode array detector (Finnigan Surveyor PDA Plus detector, Thermo Electron Corporation) and an Ion-Trap (LCQ Fleet, Thermo Scientific). Analyses were performed using a Alltech Alltima HP C₁₈ 3μ column using an injection volume of 1-4 μL, a flow rate of 0.2 mL min⁻¹ and typically a gradient (5% to 100% in 10 min, held at 100% for a further 3 min) of CH₃CN in H₂O (both containing 0.1% formic acid) at 25° C. Preparative RP-HPLC (CH₃CN/H₂O with 0.1% formic acid) was performed using a Shimadzu SCL-10A VP coupled to two Shimadzu LC-8A pumps and a Shimadzu S^{P}D-10AV VP UV-vis detector on a Phenomenex Gemini 5μ C₁₈ 110A column. Size exclusion (SEC) HPLC was carried out on an Agilent 1200 system equipped with a Gabi radioactive detector. The samples were loaded on a Superdex-200 10/300 GL column (GE Healthcare Life Sciences) and eluted with 10 mM phosphate buffer, pH 7.4, at 0.35-0.5 mL/min. The UV wavelength was preset at 260 and 280 nm. The concentration of antibody solutions was determined with a NanoDrop 1000 spectrophotometer (Thermo Fisher Scientific) from the absorbance at 322 nm and 280 nm, respectively.

Materials.

All reagents, chemicals, materials and solvents were obtained from commercial sources, and were used as received: Biosolve, Merck and Cambridge Isotope Laboratories for (deuterated) solvents; and Aldrich, Acros, ABCR, Merck and Fluka for chemicals, materials and reagents. All solvents were of AR quality.

### General Examples

The invention can be exemplified with the same combinations of TCO and diene as included in applications WO2012156919A1 (e.g. Examples 9-14) and WO2012156920A1 (e.g. Examples 8-11).

### Example 1

### Synthesis of Tetrazine Activators

For previously synthesized tetrazines see WO2012156919A1 and WO2012156920A1. Bis-pyridyl-tetrazine-NHS derivative was described in J. Nucl. Med. 2013, 54, 1989-1995.

### 3,6-dibenzyl-1,2,4,5-tetrazine (4)

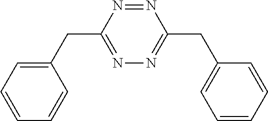 [SMILES: c1ccc(Cc2nnc(Cc3ccccc3)nn2)cc1]

Hydrazine hydrate (2.43 mL, 50.0 mmol) was added to a solution of benzyl cyanide (1.16 mL, 10.0 mmol) and ZnI₂ (160 mg, 0.5 mmol) in DMF (20 mL) and the solution was stirred overnight at 60° C. under argon. NaNO₂ (3.45 g, 50.0 mmol in 10 mL H₂O) was added dropwise to the suspension at room temperature. 1M HCl (ca. 80 mL) was added until gas formation stopped and pH=2. The mixture was extracted with CH₂Cl₂ (3×80 mL) and the combined organic fractions were dried with Na₂SO₄ and concentrated. 4 was obtained after silica gel column chromatography (EtOAc/heptanes, 1/20) as purple oil. Yield: 0.64 g (2.44 mmol, 45%). ¹H NMR (400 MHz, CDCl₃): δ 7.38-7.26 (m, 8H), 3.75 (s, 4H) ppm. ¹³C NMR (400 MHz, CDCl₃): δ 129.9, 129.1, 128.0, 127.9, 23.6 ppm. No MS data available due to poor ionization.

### Synthesis of 3,6-diisopropyl-1,2,4,5-tetrazine (5)

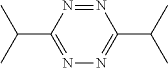

Hydrazine hydrate (13.2 mL, 312 mmol) was added to isobutyronitrile (3.59 mL, 40.0 mmol) and ZnI₂ (0.64 g, 2.0 mmol) and the mixture was stirred overnight at 60° C. under argon. NaNO₂ (13.45 g, 200 mmol in 200 mL H₂O) was added dropwise to the light colored suspension at room temperature over a cold-water bath. 1M HCl (ca. 400 mL) was added to the pink solution until gas formation stopped and pH=2. The mixture was extracted with CH₂Cl₂ (4×100 mL) and the combined organic fractions were dried with Na₂SO₄ and concentrated. 5 was obtained after silica gel column chromatography (EtOAc/hexanes, 1/9) as volatile purple oil. Yield: 3.6 g (21.4 mmol, quantitative yield). R_{f}: 0.25 (EtOAc/hexanes, 1/9). ¹H NMR (400 MHz, CDCl₃): δ 3.63 (sep, J=7.2 Hz, 2H), 1.52 (d, J=7.0 Hz, 12H) ppm. ¹³C NMR (400 MHz, CDCl₃): δ 173.8, 34.2, 21.3 ppm. ESI-MS [M+H⁺]: calc: 167.13 Da. found: 167.08 Da.

### 3,6-dimethyl-1,2,4,5-tetrazine (8)

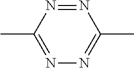

Acetamidine hydrochloride (3.97 mg; 42.0 mmol) was dissolved in water (20 mL), and hydrazine hydrate (4.0 mL; 84.0 mmol) was added. The mixture was stirred at 20° C. under an atmosphere of argon for 5 h. Water (20 mL) was added, followed by sodium nitrite (14.4 g; 210 mmol). The reaction mixture was cooled on an icebath and acidified to pH=3 by careful addition of acetic acid (15.0 g; 250 mmol). The dark pink, aqueous solution was extracted with dichloromethane (2 times 50 mL), and the combined organic layers were washed with 1 M hydrochloric acid (50 mL), dried over magnesium sulfate, and the solvent was removed by evaporation. The product was obtained as dark red crystals (750 mg; 33%). ¹H-NMR (CDCl₃): δ=3.04 (s, 6H) ppm. ¹³C-NMR (CDCl₃): δ=167.2, 21.0 ppm. GC-MS: m/z=+110 M+(calcd 110.06 for C4H₆N₄).

### 3-methyl-6-(pyridin-3-yl)-1,2,4,5-tetrazine (10)

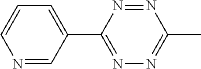

Hydrazine hydrate (2.68 mL, 55.2 mmol) was added to 3-cyanopyridine (500 mg, 4.8 mmol), acetamidine hydrochloride (2.00 g, 21.2 mmol) and sulfur (78 mg, 2.4 mmol) and the mixture was stirred overnight under argon at room temperature. The reaction mixture was concentrated and suspended in a mixture of THF (10 mL) and AcOH (12 mL) over a cold-water bath. NaNO₂ (2.76 g, 40.0 mmol in 10 mL H₂O) was added dropwise and the mixture was stirred for another 5 minutes. H₂O (80 mL) and CHCl₃ (100 mL) were added and the layers were separated. The organic layer was washed with H₂O (2×100 mL), dried with Na₂SO₄ and concentrated. Silica gel column chromatography (acetone/hexanes, 1/4) yielded 10 contaminated with a small amount of the bis-pyridyl side product. Recrystallization from EtOAc yielded 10 as long needles (70 mg, 0.40 mmol, 8%). Concentration of the EtOAc filtrate yielded another crop (170 mg) of almost pure 10. ¹H NMR (400 MHz, CDCl₃): δ 9.80 (dd, J_{1=0.8} Hz, J_{2=1.9} Hz), 8.88-8.84 (m, 2H), 7.55 (m, 1H), 3.14 (s, 3H) ppm. ¹³C NMR (400 MHz, CDCl₃): δ 168.0, 163.1, 153.2, 149.3, 135.1, 127.9, 123.9, 21.3 ppm. ESI-MS [M+H+] calc: 174.08 Da. found: 174.08 Da.

### 3-methyl-6-(pyridin-4-yl)-1,2,4,5-tetrazine (11)

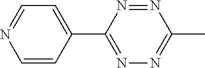

Hydrazine hydrate (2.68 mL, 55.2 mmol) was added to 4-cyanopyridine (500 mg, 4.8 mmol), acetamidine hydrochloride (2.00 g, 21.2 mmol) and sulfur (78 mg, 2.4 mmol) and the mixture was stirred overnight under argon at room temperature. The reaction mixture was concentrated and suspended in a mixture of THF (10 mL) and AcOH (12 mL) over a cold-water bath. NaNO₂ (2.76 g, 40.0 mmol in 10 mL H₂O) was added dropwise and the mixture was stirred for another 5 minutes. H₂O (80 mL) and CHCl₃ (100 mL) were added and the layers were separated. The organic layer was washed with H₂O (2×100 mL), dried with Na₂SO₄ and concentrated. Silica gel column chromatography (acetone/hexanes, 1/4) yielded 11 with a ca. 20% contamination of a thiadiazole compound. The crude material (220 mg) was recrystallized from diisopropylether to yield 11 as pink crystals. Yield: 135 mg (0.78 mmol, 16%). R_{f}: 0.07 (acetone/hexanes, 1/4). ¹H NMR (400 MHz, CDCl₃): δ 8.91 (dd, J_{1=1.5} Hz, J_{2=4.7} Hz, 2H), 8.44 (dd, J_{1=1.8} Hz, J_{2=4.5} Hz, 2H), 3.17 (s, 3H) ppm. ¹³C NMR (400 MHz, CDCl₃): δ 168.5, 163.0, 151.1, 139.2, 121.2, 21.4 ppm. ESI-MS [M+H⁺]: calc: 174.08 Da. found: 174.08 Da.

### 3-methyl-6-(3-methylpyridin-2-yl)-1,2,4,5-tetrazine (12)

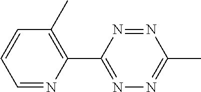

Hydrazine hydrate (2.76 mL, 56.2 mmol) was added to 3-methylpicolinonitrile (0.57 g, 4.8 mmol), acetamidine hydrochloride (2.00, 21.2 mmol) and sulfur (155 mg, 4.8 mmol) and the mixture was stirred under argon at room temperature for 40 hours. EtOH (10 mL) was added and the mixture was filtered. The filtrate was concentrated and suspended in a mixture of THF (10 mL) and AcOH (12 mL) over a cold-water bath. NaNO₂ (2.76 g, 40.0 mmol in 10 mL H₂O) was added dropwise and the mixture was stirred for another 5 minutes. H₂O (80 mL) and CHCl₃ (100 mL) were added and the layers were separated. The organic layer was washed with H₂O (2×100 mL), dried with Na₂SO₄ and concentrated. Silica gel column chromatography (acetone/hexanes, 1/4) yielded 12 as purple liquid. Yield: 110 mg (0.59 mmol, 12%). ¹H NMR (400 MHz, CDCl₃): δ 8.73 (dd, J_{1=0.8} Hz, J_{2=4.6} Hz, 1H), 7.76 (ddd, J_{1=0.8} Hz, J₂=1.5, J₃=7.8 Hz, 1H), 7.43 (dd, J₁=4.7 Hz, J₂=7.8 Hz, 1H), 3.17 (s, 3H), 2.60 (s, 3H) ppm. ¹³C NMR (400 MHz, CDCl₃): δ 167.3, 166.0, 149.8, 148.0, 139.7, 134.5, 125.2, 21.4, 19.8 ppm. ESI-MS [M+H⁺]calc: 188.09 Da. found: 188.08 Da.

### 3-methyl-6-phenyl-1,2,4,5-tetrazine (14)

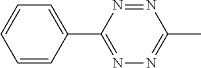

Hydrazine hydrate (3.24 mL, 66.7 mmol) was added to benzonitrile (600 mL, 5.8 mmol), acetamidine hydrochloride (2.41 g, 25.5 mmol) and sulfur (94 mg, 2.9 mmol) and the mixture was stirred overnight under argon at room temperature. The reaction mixture was concentrated and suspended in a mixture of THF (10 mL) and AcOH (12 mL) over a cold-water bath. NaNO₂ (3.33 g, 28.3 mmol in 10 mL H₂O) was added dropwise and the mixture was stirred for another 5 minutes. H₂O (50 mL) and CHCl₃ (100 mL) were added and the layers were separated. The organic layer was washed with H₂O (2×70 mL), dried with Na₂SO₄ and concentrated. Silica gel column chromatography (acetone/hexanes, 1/4) yielded 14 with some contamination (ca. 75 mg). The crude product could not be purified by recrystallization from numerous solvents. ¹H NMR (400 MHz, CDCl₃): δ 8.59 (dd, J_{1=1.6} Hz, J₂=8.2 Hz, 2H), 7.68-7.60 (m, 2H), 7.50-7.46 (m, 1H), 3.10 (s, 3H) ppm. ¹³C NMR (400 MHz, CDCl₃): δ 167.3, 164.2, 132.6, 131.8, 129.3, 127.9, 21.2 ppm. MALDI-TOF-MS: [M+H⁺]: calc: 173.08 Da. found 173.30 Da.

### Example 2

### TCO Synthesis

The following TCO constructs have been prepared according to WO2012156920A1:

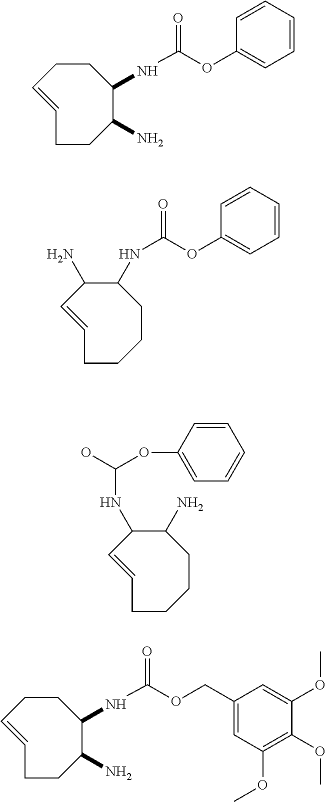

3-PNP-TCO was synthesized following WO2012156919A1.

### Axial-TCO-1-Doxorubicin

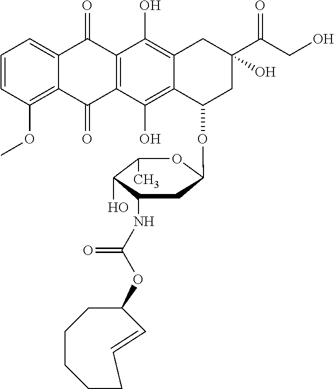

The synthesis of Axial—TCO-1-Doxorubicin is described in WO2012156919A1.

### Synthesis of (E)-cyclooct-2-enyl naphthalen-1-ylmethylcarbamate

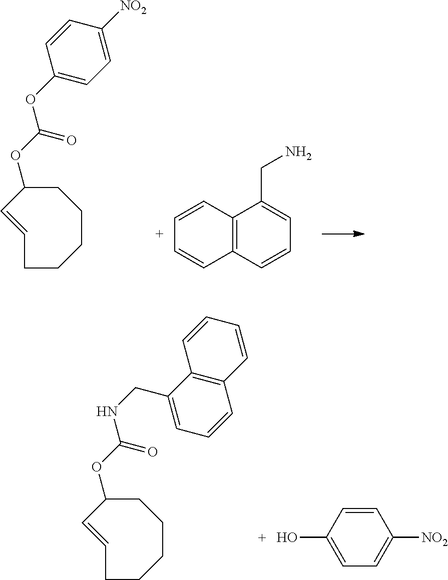 [SMILES: O=C(Oc1ccc([N+](=O)[O-])cc1)OC1/C=C/CCCCC1 ; NCc1cccc2ccccc12 ; O=C(NCc1cccc2ccccc12)OC1/C=C/CCCCC1 ; O=[N+]([O-])c1ccc(O)cc1]

3-PNP-TCO (41.9 mg; 1.44*10⁻⁴ mol) was dissolved in dichloromethane (1.5 mL), and DIPEA (55.7 mg; 4.32*10⁻⁴ mol) and 1-naphthylmethylamine (27.2 mg; 1.73*10⁻⁴ mol) were added. The reaction mixture was stirred at 20° C. under and atmosphere of argon and slowly turned yellow. After 20 h the solvent was removed by evaporation in vacuo, and the mixture was redissolved in dichloromethane and washed with subsequently, 1 M aqueous sodium hydroxide (5 times 2.5 mL) and 1 M aqueous citric acid (2 times 1.5 mL). The organic layer was dried over sodium sulfate, filtered, and evaporated to dryness. The product was further purified by preparative RP-HPLC, and isolated by lyophilization, to yield a white powder (32.0 mg; 72%). ¹H-NMR (CDCl₃): δ=8.04 (d, 1H), 7.89 (d, 1H), 7.81 (d, 1H), 7.54 (m, 2H), 7.45 (m, 2H), 5.79 (m, 1H), 5.56 (d, 1H), 5.40 (m, 1H), 5.03 (br. s, 1H), 4.85 (m, 2H), 2.44 (m, 1H), 2.2-1.6 (br. m, 6H), 1.43 (m, 1H), 1.02 (m, 1H), 0.79 (m, 1H) ppm. ¹³C-NMR (CDCl₃): δ=131.7, 131.4, 128.8, 128.5, 126.5, 126.4, 125.9, 125.4, 123.5, 74.2, 43.2, 40.7, 35.9, 29.1, 29.0, 24.1 ppm. FT-IR (ATR): v=3322, 2927, 2857, 1692, 1533, 1258, 1070, 1025, 987 cm′. LC-MS: m/z=+310.25 [M+H]+(calcd 309.17 for C₂₀H₂₃NO₂).

### Axial-(E)-cyclooct-2-en-1-yl 4-nitrobenzoate

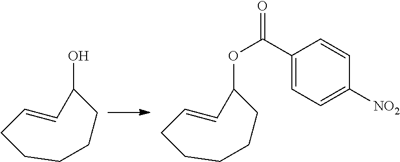 [SMILES: OC1/C=C/CCCCC1 ; O=C(OC1/C=C/CCCCC1)c1ccc([N+](=O)[O-])cc1]

Axial-(E)-cyclooct-2-en-1-ol (152 mg, 1.20 mmol) was dissolved in 10 mL dichloromethane. 4-(N,N-dimethylamino)pyridine (306 mg, 2.50 mmol) was added and the solution was cooled in an ice-bath. A solution of 4-nitrobenzoyl chloride (201 mg, 1.08 mmol) in 5 mL dichloromethane was added in portions over a 5 min period. The solution was stirred for 3 days. The solvent was partially removed by rotary evaporation. The remaining solution (a few mL) was chromatographed on 19 g silica, using dichloromethane as the eluent. The product fractions were rotary evaporated yielding a colourless solid (144 mg, 0.52 mmol, 48%). ¹H-NMR (CDCl₃): δ 8.4-8.2 (m, 4H), 5.9 (m, 1H), 5.6 (m, 2H), 2.2 (dd, 1H), 2.5 (m, 1H), 2.15-1.7 (m, 6H), 1.55 (m, 1H), 1.2 (dt, 1H), 0.9 (dt, 1H).

### Equatorial-(E)-cyclooct-2-en-1-yl 4-nitrobenzoate

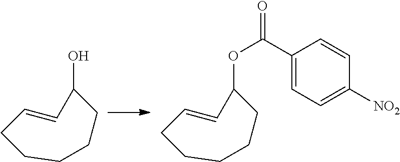 [SMILES: OC1/C=C/CCCCC1 ; O=C(OC1/C=C/CCCCC1)c1ccc([N+](=O)[O-])cc1]

Equatorial-(E)-cyclooct-2-en-1-ol (154 mg, 1.22 mmol) was dissolved in 10 mL dichloromethane. 4-(N,N-dimethylamino)pyridine (300 mg, 2.46 mmol) was added and the solution was cooled in an ice-bath. A solution of 4-nitrobenzoyl chloride (268 mg, 1.44 mmol) in 5 mL dichloromethane was added in portions over a 5 min period. The solution was stirred for 4 days. The solvent was removed by rotary evaporation and the residue was chromatographed on 19 g silica, using dichloromethane as the eluent. The product fractions were rotary evaporated yielding a colourless solid. ¹H-NMR (CDCl₃): δ 8.4-8.1 (m, 4H), 5.9 (m, 1H), 5.7 (m, 1H), 5.4 (m, 1H), 2.5 (m, 1H), 2.3 (m, 1H), 2.1-1.8 (m, 3H), 2.8-2.4 (m, 4H), 1.8-1.4 (m, 4H), 1.0-0.8 (m, 1H).

### (E)-3-phenoxycyclooct-1-ene

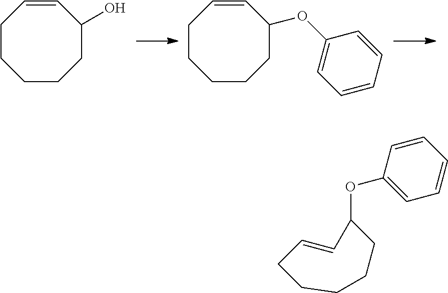 [SMILES: OC1/C=C\CCCCC1 ; C1=C\C(Oc2ccccc2)CCCCC/1 ; C1=C/C(Oc2ccccc2)CCCCC/1]

Cyclooct-2-en-1-ol (5.002 g, 39.64 mmol) was dissolved in 100 mL THF. Phenol (3.927 g, 41.78 mmol) was added to the solution. Triphenylphosphine (10.514 g, 40.01 mmol) was added and the resulting solution was cooled in an ice-bath. A solution of diethyl azodicarboxylate (6.975 g, 40.01 mmol) in 50 mL THF was added over a 30 min period. The reaction mixture was stirred for 24 h and then rotary evaporated. The residue was stirred with heptane, the mixture was filtered and the filtrate was rotary evaporated. The residue was chromatographed on 50 g silica, using heptane as eluent. Product fractions were rotary evaporated and the residue was stirred with methanol until homogeneous, then filtered, and rotary evaporated. The residue was purified by Kugelrohr distillation to yield the product as an oil (3.5 g, 17.33 mmol, 44%). 3-phenoxycyclooctene (5.5 g, 27.23 mmol) was dissolved in heptane-ether (ca. 1/2). The solution was irradiated for 7 days while the solution was continuously flushed through a 42 g silver nitrate impregnated silica column (containing ca. 4.2 g silver nitrate). The column was rinsed twice with TBME, then with TBME containing 5% methanol, then with TBME containing 10% MeOH. The product fractions were washed with 100 mL 15% ammonia (the same ammonia being used for each fraction), then dried and rotary evaporated. The column material was stirred with TBME and 15% ammonia, then filtered, and the layers were separated. The organic layer was dried and rotary evaporated. The first two TBME fractions were combined, and all other fractions were separately rotary evaporated, then examined for the presence of the product (none of the fractions contained a pure trans-cyclooctene isomer, however). The product fractions were combined and chromatographed on 102 g silica, using heptane as the eluent. The first fractions yielded the pure minor (believed to be axial) isomer as an oil (144 mg, 0.712 mmol, 2.6%). The next fractions contained a mixture of minor and major isomer. Pure major (believed to be equatorial) isomer was eluted last, yielding a colourless solid (711 mg, 3.52 mmol, 13%). (Z)-3-phenoxycyclooct-1-ene: ¹H-NMR (CDCl₃): δ 7.25 (m, 2H), 6.9 (m, 3H), 5.7 (m, 1H), 5.5 (m, 1H), 5.1 (m, 1H), 2.5-2.0 (m, 3H), 1.3-1.9 (m, 7H). (E)-3-phenoxycyclooct-1-ene (axial isomer): ¹H-NMR (CDCl₃): δ 7.25 (m, 2H), 6.9 (m, 3H), 5.9 (m, 1H), 5.6 (m, 1H), 4.9 (s, 1H), 2.4 (m, 1H), 2.2 (m, 1H), 2.0-0.8 (m, 8H). (E)-3-phenoxycyclooct-1-ene (equatorial isomer): ¹H-NMR (CDCl₃): δ 7.25 (m, 2H), 6.9 (m, 3H), 5.9 (m, 1H), 5.55 (m, 1H), 4.8 (m, 1H), 2.45-2.25 (m, 2H), 2.05-1.4 (m, 6H), 1.0-0.8 (m, 2H)

### Axial (E)-cyclooct-2-en-1-yl 2-phenylacetate

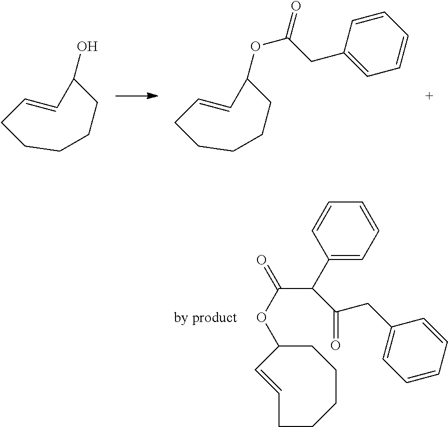 [SMILES: OC1/C=C/CCCCC1 ; O=C(Cc1ccccc1)OC1/C=C/CCCCC1 ; "by product" ; O=C(Cc1ccccc1)C(C(=O)OC1/C=C/CCCCC1)c1ccccc1]

Axial (E)-cyclooct-2-en-1-ol (102 mg, 0.81 mmol) was dissolved in 7.5 mL dichloromethane with 4-(N,N-dimethylamino)pyridine (303 mg, 2.70 mmol). A solution of phenylacetyl chloride (155 mg, 1.00 mmol) in 2.5 mL dichloromethane was added in portions over a 5 min period to the ice-cooled solution. The reaction mixture was stirred for 4 days, then washed with water. The aqueous layer was extracted with 10 mL dichloromethane. The combined organic layers where dried and rotary evaporated, followed by chromatography yielding a colourless powder (22 mg) which was identified as the depicted byproduct.

### Axial-(E)-3-(benzyloxy)cyclooct-1-ene

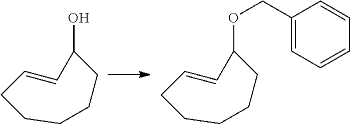 [SMILES: OC1/C=C/CCCCC1 ; C1=C/C(OCc2ccccc2)CCCCC/1]

Axial (E)-cyclooct-2-en-1-ol (131 mg, 1.04 mmol) was dissolved in 5 mL THF. Sodium hydride (60% dispersion in oil, 80 mg, 2 mmol) was added. The mixture was stirred for 5 min, then heated at 55° C. for 1 h, and then stirred at rt for 4 h. Benzyl bromide (210 μL, 300 mg, 1.9 mmol) was added in 5 small portions. The reaction mixture was stirred for 4 days, after which 10 mL water was added carefully. The mixture was extracted with 2×10 mL dichloromethane and the successive organic layers were washed with 10 mL water, dried and rotary evaporated. The residue was heated at ca. 40° C. under high vacuum in order to remove most of the benzyl bromide. The residue was purified by chromatography on 20 g silica using heptane as eluent, followed by elution with toluene. The latter solvent eluted the product. The product fractions were rotary evaporated, leaving a colourless oil, which contained traces of dibenzyl ether (69 mg, 0.32 mmol, 31%). ¹H-NMR (CDCl₃): δ 7.4-7.2 (m, 5H), 6.0 (m, 1H), 5.45 (d, 1H), 4.7-4.4 (dd, 2H), 4.2 (s, 1H), 2.5 (m, 1H), 2.2-1.8 (m, 4H), 1.7-1.5 (m, 3H), 1.3-1.1 (m, 1H), 0.8 (m, 1H)

### Axial-(E)-2,5-dioxopyrrolidin-1-yl 5-((((2,5-dioxopyrrolidin-1-yl)oxy)carbonyl)oxy)-1-methylcyclooct-3-enecarboxylate TCO-2

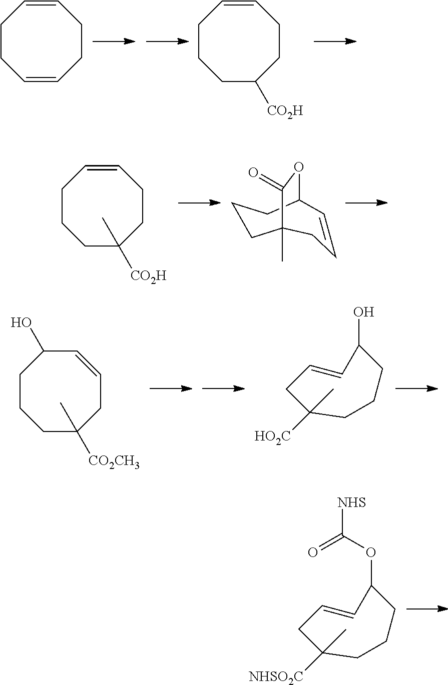

### (Z)-5-bromocyclooct-1-ene

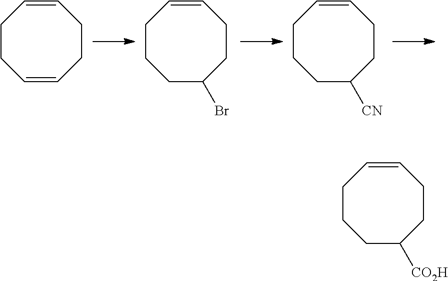 [SMILES: C1=C\CC/C=C\CC/1 ; BrC1CC/C=C\CCC1 ; N#CC1CC/C=C\CCC1 ; O=C(O)C1CC/C=C\CCC1]

1,5-cyclooctadiene (225 mL, 1.83 mol) was added to ice-cooled 310 mL 33% hydrogen bromide in acetic acid over a 30 min period at ca. 10° C. The mixture was stirred for 2 days, then 300 mL water was added, and the mixture was extracted with 2×300 mL pentane containing some TBME. The successive organic layers were washed with 75 mL water and with 75 mL sodium bicarbonate solution. Drying and rotary evaporation left 325 g residue which was used as such in the next step.

### (Z)-cyclooct-4-enecarbonitrile

A mixture of 700 mL DMSO and sodium cyanide (117.3 g, 2.39 mol) was heated to 90° C. The bromide obtained above was added over a 4 h period at 90-96° C. The mixture was subsequently heated at 98° C. for 16 h, then it was cooled and 200 mL water was added during this cooling process. The mixture was extracted with 3×300 mL pentane containing some TBME. Washing with 50 mL water, drying and rotary evaporation resulted in 170 g residue which was used as such in the next step. See J. Org. Chem. 1988, 53, 1082 for a similar procedure.

### (Z)-cyclooct-4-enecarboxylic acid

The product obtained above was treated with 100 mL ethanol, 160 mL 35% hydrogen peroxide, and 400 mL 30% sodium hydroxide solution, via the method described by D. Hartley in J. Chem. Soc. 1962, 4722. After acidification, further workup and Kugelrohr distillation, the distillate (94.4 g) appeared to be mainly the starting nitrile. This distillate, combined with ca. 25 g of the solid residue from the Kugelrohr distillation, was stirred with 400 mL ethanol. Potassium hydroxide (155 g, 2.35 mol) was added, and the mixture was cooled with cold water (reaction mixture attained 40° C.). When the temperature had dropped to 25° C., 35 mL water was added, followed by the portion-wise addition of 140 mL 35% hydrogen peroxide (foaming, temperature around 30° C.). After the addition was complete and the temperature had dropped, the cooling-bath was removed and replaced by a heating mantle. The mixture was warmed up slowly, resulting in an exothermal reaction and foaming. Hereby the temperature gradually reached 63° C. (some cooling was necessary). When the temperature had decreased to 55° C., 100 mL 30% sodium hydroxide solution was added. The mixture was then heated for 4 h, while distilling off ca. 350 mL of solvent. Another 30 mL 30% sodium hydroxide solution was added and the mixture was heated under reflux for 10 h. The reaction mixture was cooled to rt, 400 mL heptane was added and the layers were separated. The organic layer was washed with a small amount of water. The combined aqueous layers were acidified with conc. hydrochloric acid and extracted with 3×250 mL TBME. Drying, rotary evaporation and Kugelrohr distillation gave 109.77 g of the desired acid (0.713 mol, 39% yield based on 1,5-cyclooctadiene).

### (Z)-1-methylcyclooct-4-enecarboxylic acid

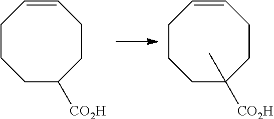

A mixture of diisopropylamine (90.2 g, 0.893 mol) and 300 mL THF was cooled below −20° C. n-Butyllithium in hexanes (2.5 N, 360 mL, 0.900 mol) was added in a slow stream, keeping the temperature below −20° C. The solution was stirred for 15 min, then cooled to −50° C. (Z)-cyclooct-5-enecarboxylic acid (54.0 g, 0.351 mol), dissolved in 150 mL THF, was added over a 20 min period at temperatures between −50 and −25° C. The mixture was stirred for an additional 40 minutes, allowing the temperature to rise to −5° C. The mixture was subsequently heated for 3 h at 50° C., then cooled to −50° C. Iodomethane (195.5 g, 1.377 mol) was added over a 20 min period at temperatures between −50 and −30° C. The mixture was stirred overnight, heated for 1 h at 40° C., then rotary evaporated in order to remove most of the solvents. Toluene (250 mL) was added to the residue, followed by 500 mL dilute hydrochloric acid. The layers were separated and the organic layer was washed with 100 mL 2 N hydrochloric acid. The successive aqueous layers were extracted with 2×250 mL toluene. The organic layers were dried and rotary evaporated. The residue was purified by Kugelrohr distillation to yield 59.37 g of the methylated acid (0.353 mol, 100%), which was sufficiently pure to be used as such in the next step.

¹H-NMR (CDCl₃): δ 5.75-5.60 (m, 1H), 5.55-5.40 (m, 1H), 2.4-1.5 (m, 10H), 1.27 (s, 3H). ¹³C-NMR (CDCl₃): δ 185.5 (C═O), 131.9 (═CH), 126.5 (═CH), 46.2, 35.3, 32.3, 27.1 (CH₃), 26.1, 24.8, 24.7.

### (1R,5R)-5-methyl-9-oxabicyclo[3.3.2]dec-7-en-10-one

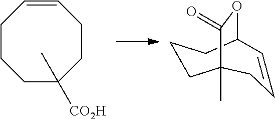

To a mixture of the methylated acid (42.0 g, 0.25 mol), 300 mL dichloromethane, and 300 mL water sodium bicarbonate was added (68.9 g, 0.82 mol). The mixture was stirred for 10 min, then it was cooled in ice. A mixture of potassium iodide (125.2 g, 0.754 mol) and iodine (129 g, 0.508 mol) was added over a 1 h period in 6 equal portions. The reaction mixture was stirred for 31/2 h. Sodium bisulfite was added slowly, until the dark colour had disappeared. The layers were separated and the cloudy aqueous layer was extracted with 2×250 mL dichloromethane. Drying and rotary evaporation gave the desired iodolactone. ¹H-NMR (CDCl₃, product signals): δ 5.65-5.5 (m, 2H), 4.8 (dt, 1H), 3.95 (dt, 1H), 2.6-1.95 (m, 8H).

The iodolactone was dissolved in 250 mL toluene, and DBU (65.2 g, 0.428 mol) was added. The mixture was allowed to stand overnight, after which it was heated under reflux for 75 min (NMR indicated full conversion). After cooling the reaction mixture, it was washed with 150 and 100 mL water. The successive aqueous layers were extracted with 250 mL toluene. The organic layers were dried and rotary evaporated and the residue was purified by Kugelrohr distillation to yield 38.86 g of the bicyclic olefin (0.234 mol, 94%, containing a trace of toluene). ¹H-NMR (CDCl₃): δ 5.95-5.85 (m, 1H), 5.45-5.35 (dm, 1H), 5.05 (bs, 1H), 2.5-2.3 (m, 1H), 2.2-2.0 (m, 2H), 1.95-1.6 (m, 5H), 1.27 (s, 3H). ¹³C-NMR (CDCl₃): δ 177.2 (C═O), 129.1 (═CH), 127.9 (═CH), 79.2 (CH), 45.2, 43.0, 31.9, 29.5 (CH₃), 26.6, 24.0.

### (Z)-methyl 5-hydroxy-1-methylcyclooct-3-enecarboxylate

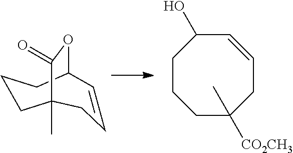

The bicyclic olefin obtained above (38.86 g, 0.234 mol), plus another batch of 1.5 g bicyclic olefin, was stirred for 64 h at 25-30° C. with 250 mL methanol and potassium bicarbonate (100.0 g, 1.0 mol). Another 50.0 g potassium bicarbonate (0.5 mol) was added because NMR indicated the presence of ca. 35% starting olefin. The mixture was stirred for an additional 64 h, but the amount of starting material remained unchanged. Filtration, washing with methanol and rotary evaporation of the filtrate gave a residue, which was chromatographed on 200 g silica using dichloromethane as the eluent. The starting olefin eluted first, then a mixture of starting olefin and product eluted. Further elution with dichloromethane/methanol gave 6.69 g of product, contaminated with ca. 15% of starting olefin, and then 17.53 g of pure product (total 0.117 mmol, 48%).

¹H-NMR (CDCl₃): δ 5.6-5.5 (m, 1H), 5.35-5.25 (m, 1H), 5.0-4.85 (m, 1H), 3.63 (s, 3H), 2.90 (d, 1H, OH), 2.35-1.90 (m, 5H), 1.75-1.45 (m, 3H), 1.20 (s, 3H). ¹³C-NMR (CDCl₃): δ 178.8 (C═O), 132.7 (═CH), 129.0 (═CH), 68.0 (CH), 52.0 (CH₃), 46.1, 35.9, 33.7, 30.4, 26.8, 24.7 (CH₃).

### (E)-methyl 5-hydroxy-1-methylcyclooct-3-enecarboxylate

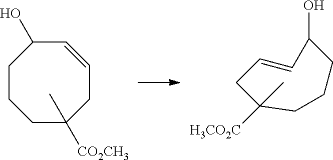

The two portions of hydroxy ester obtained above, plus 2.29 g of hydroxy ester from another experiment (total amount 26.51 g, 133.8 mmol) were mixed with 25.0 g methyl benzoate and heptane/ether (ca. 4/1). The solution was irradiated, the irradiated solution being continuously flushed through a silver nitrate impregnated silica column (213.6 g, containing ca. 126 mmol silver nitrate). During the irradiation process some solvent was lost due to evaporation; this solvent was replaced by ether. The irradiation and flushing were stopped when the irradiated solution contained hardly any starting material. The silica column was successively flushed with 600 mL TBME, 500 mL TBME/5% methanol, 500 mL TBME/10% methanol, and 500 mL TBME/20% methanol. The first 3 eluates were rotary evaporated. The first eluate contained methyl benzoate and the starting hydroxy ester in a ca. 2/3 ratio. The fourth eluate was washed with 300 mL 10% ammonia solution, then dried and rotary evaporated (axial/equatorial ratio of the trans-cycloctene was ca. 5/4). The residues from the second and third eluate were combined, dissolved in TBME and washed with the ammonia layer of above. Drying and rotary evaporation gave a residue which consisted of the axial/equatorial isomers of the trans-cycloctene in a ratio of ca. 5/4. The residual column material was stirred with TBME, 100 mL water and the ammonia layer of above. Filtration, layer separation, drying and rotary evaporation gave a residue. The process was repeated twice to give a residue which consisted of the axial/equatorial isomers of the trans cycloctenes in a ratio of ca. 1/7. All fractions of the trans cyclooctenes were combined to give a total yield of 19.1 g (96.5 mmol, 72%). Note: The axial/equatorial assignment is based on the the stereochemistry of the hydroxy group, in similar fashion as for trans-cycloocten-2-ol. In both isomers the hydroxy and methylester substituents are positioned cis relative to each other. In the axial isomer, these cis-positioned substituents are both in the axial position.

¹H-NMR (CDCl₃) (mixture of isomers): axial isomer: δ 5.8 (m, 1H), 5.35 (m, 1H), 4.2 (m, 1H), 3.72 (s, 3H), 2.7 (m, 1H), 2.3-1.7 (m, 6H), 1.5 (m, 1H), 1.3 (m, 1H), 1.19 (s, 3H). ¹³C-NMR (CDCl₃): δ 177.6 (C═O), 136.1 (═CH), 132.3 (═CH), 74.8 (CH), 51.5 (CH₃), 47.5, 46.0, 39.9, 38.9, 34.8 (CH₃), 31.0.

¹H-NMR (CDCl₃) (mixture of isomers): equatorial isomer: δ 6.05 (m, 1H), 5.6 (dd, 1H), 4.45 (bs, 1H), 3.62 (s, 3H), 2.35-1.7 (m, 8H), 1.5 (m, 1H), 1.08 (s, 3H). ¹³C-NMR (CDCl₃): δ 180.7 (C═O), 135.2 (═CH), 130.3 (═CH), 69.6 (CH), 52.1 (CH₃), 44.9, 44.7, 38.3, 30.9, 29.8, 18.3 (CH₃).

### Axial-(E)-5-hydroxy-1-methylcyclooct-3-enecarboxylic acid

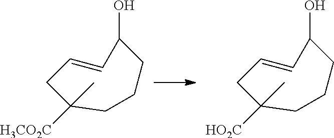

A solution of 1.60 g potassium hydroxide in 5 mL water was added over a 5 min period to a water-cooled solution of the trans-cyclooctene ester isomer mixture (0.49 g, 2.47 mmol, ratio of the axial/equatorial isomer ca. 21/2/1) in 11 mL methanol. The solution was stirred for 18 h at 28° C. 15 mL water was added and the mixture was extracted with 2×30 mL TBME. The combined organic layers were washed with 10 mL water, then dried and rotary evaporated to give the non-hydrolyzed equatorial ester. The combined aqueous layers were treated with 30 mL TBME, and then with 4.5 g citric acid. The layers were separated and the aqueous layer was extracted with 30 mL TBME. The organic layers were dried and rotary evaporated at 55° C. to afford 0.34 g (1.85 mmol, 75%) of the pure axial isomer of the trans-cyclooctene acid. ¹H-NMR (CDCl₃): δ 6.15-5.95 (m, 1H), 5.6 (d, 1H), 4.45 (bs, 1H), 2.4-1.7 (m, 7H), 1.6 (dd, 1H), 1.18 (s, 3H). ¹³C-NMR (CDCl₃): δ 185.4 (C═O), 134.8 (═CH), 130.7 (═CH), 69.8 (CH), 44.8, 38.2, 31.0, 29.8 (CH₂), 18.1 (CH₃).

Note: The hydrolysis of the axial/equatorial ester appears to be extremely selective. Whereas the axial isomer hydrolyzes surprisingly easily at rt, the major isomer remains unaffected, thus enabling an straightforward separation between both isomers (the equatorial isomer hydrolyzes upon overnight heating at ca. 60° C.). In both isomers the hydroxy and carboxylic substituents are positioned cis relative to each other. In the axial isomer, these cis-positioned substituents are both in the axial position.

### Axial-(E)-2,5-dioxopyrrolidin-1-yl 5-((((2,5-dioxopyrrolidin-1-yl)oxy)carbonyl)oxy)-1-methylcyclooct-3-enecarboxylate TCO-2

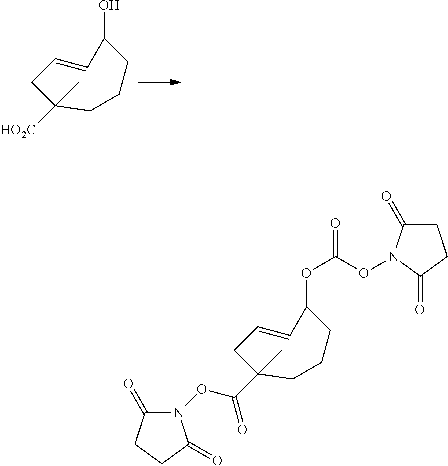 [SMILES: CC1(C(=O)O)C/C=C/C(O)CCC1 ; CC1(C(=O)ON2C(=O)CCC2=O)C/C=C/C(OC(=O)ON2C(=O)CCC2=O)CCC1]

To a solution of Axial-(E)-5-hydroxy-1-methylcyclooct-3-enecarboxylic acid obtained above (375 mg, 2.04 mmol) in 10.1 g acetonitrile there was added N,N-diisopropylethylamine (1.95 g, 15.07 mmol), followed by N,N′-disuccinimidyl carbonate (2.25 g, 8.79 mmol). The mixture was stirred for 3 days at rt, and subsequently rotary evaporated at 55° C. The residue was chromatographed on 20 g silica, elution being done with dichloromethane, followed by elution with dichloromethane containing some TBME. The latter solvent mixture eluted the product. The product fractions were combined and rotary evaporated. The resulting residue was stirred with TBME until a homogeneous suspension was obtained. Filtration and washing gave 400 mg of product. ¹H-NMR (CDCl₃): δ 6.15-6.0 (m, 1H), 5.6 (dd, 1H), 5.25 (bs, 1H), 2.8 (2s, 8H), 2.5-1.85 (m, 8H), 1.25 (s, 3H).

### Axial-TCO-2-Doxorubicin

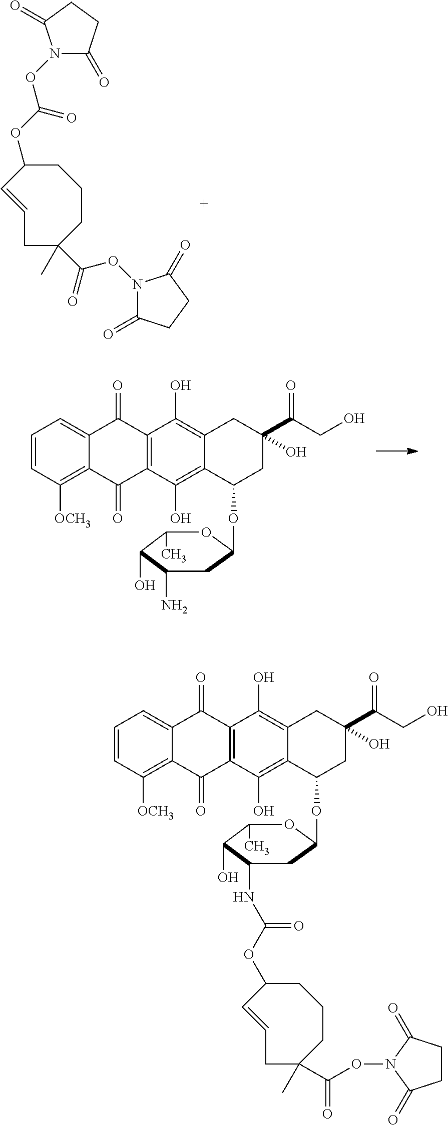

Doxorubicin hydrochloride (133 mg; 2.30*10⁻⁴ mol) and TCO-2 (97.0 mg; 2.30*10⁻⁴ mol) were dissolved in DMF (5 mL), and DIPEA (148 mg; 1.15*10⁻³ mol) was added. The solution was stirred under an atmosphere of argon at 20° C. for 18 h. Acetonitrile (6.5 mL), formic acid (0.2 mL), and water (6.5 mL), were added and the suspension was filtered. The filtrate was purified by preparative RP-HPLC (50 v % acetonitrile in water, containing 0.1 v % formic acid). The product was isolated by lyophilization, dissolved in chloroform (3 mL), and precipitated in diethyl ether (20 mL), to yield 134 mg of an orange powder (68%). ¹H-NMR (CDCl₃): =13.97 (s, 1H), 13.22 (s, 1H), 8.03 (d, J=7.9 Hz, 1H), 7.78 (t, J=8.0 Hz, 1H), 7.38 (d, J=8.6 Hz, 1H), 5.85 (m, 1H), 5.59 (m, 1H), 5.51 (s, 1H), 5.29 (s, 1H). 5.16 (d, J=8.4 Hz, 1H), 5.12 (s, 1H), 4.75 (d, J=4.8 Hz, 2H), 4.52 (d, J=5.8 Hz, 1H), 4.15 (q, J=6.5 Hz, 1H), 4.08 (d, J=3.6 Hz, 3H), 3.87 (m, 1H), 3.69 (m, 1H), 3.26 (d, J=18.8 Hz, 1H), 3.00 (m, 2H), 2.81 (s, 4H), 2.4-1.7 (br. m, 13H), 1.62 (s, 2H), 1.30 (d, J=6.5 Hz, 3H), 1.23 (s, 3H) ppm. ¹³C-NMR (CDCl₃): =213.89, 187.07, 186.68, 174.30, 169.27, 161.03, 156.15, 155.64, 154.66, 135.73, 135.49, 133.58, 131.70, 131.10, 120.88, 119.83, 118.43, 111.58, 111.40, 100.73, 72.09, 69.65, 67.28, 65.54, 56.67, 46.87, 44.38, 35.75, 34.00, 30.49, 30.39, 30.20, 25.61, 17.92, 16.84 ppm. LC-MS: m/z=+873.42 [M+Na]⁺, −849.58 [M−H]⁻(calcd 850.28 for C₄₂H₄₆N₂O₁₇).

### Example 3

### Tetrazine Induced Release of Doxorubicin from TCO-1-Doxorubicin

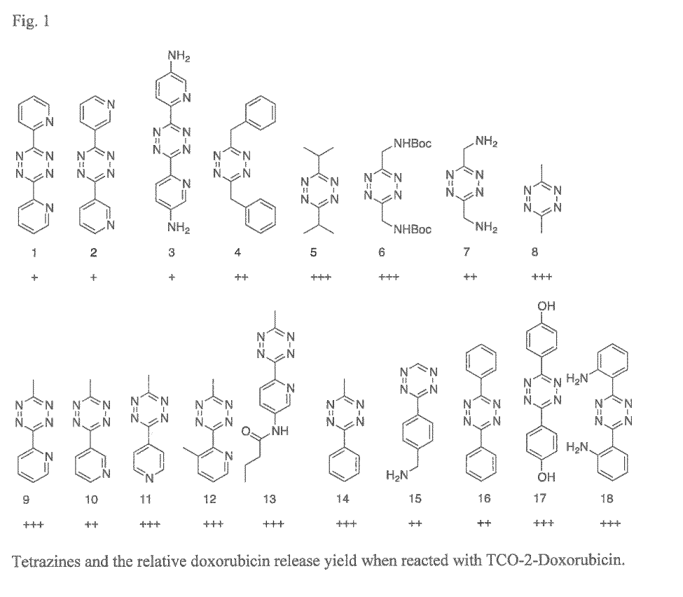

The tetrazines featured in FIG. 1 were tested with respect to their ability to release doxorubicin from TCO-2-doxorubicin. It shall be understood that the tetrazine-induced release in this experiment can be considered representative of the cleavage of D^{D}-M^{M} constructs. The relative release yield for each tetrazine is given in FIG. 1 (+++=highest).

PBS/MeCN (1 mL, 3/1), preheated at 37° C. and TCO-2-doxorubicin (10 μL of a 2.5 mM solution in DMSO, 1 eq.) were added to a preheated injection vial. Tetrazine (10 μL of a 25 mM solution in DMSO, 10 eq.) was added and the vial was vortexed. After incubation for 1 hour at 37° C., the vial was placed in LC-MS autosampler at 10° C. LC-MS analysis was performed using a 5% to 100% H₂O/MeCN gradient over 11 minutes with a C18 reverse-phase column at 35° C. A control sample containing only TCO-2-doxorubicin (1 eq), as well as a sample containing only doxorubicin (1 eq.), was analyzed under the same conditions. All tetrazine containing samples were measured twice and the doxorubicin control sample was run after every three other samples during an overnight program. The peak area of released dox was divided by the peak area of TCO-2-doxorubicin or doxorubicin reference signals and multiplied by 100 to calculate the percentage of release. The calculated percentage of release was corrected when it was observed that the TCO-2-doxorubicin was not fully converted to inv-DA adduct(s). This was done by quantification of remaining dox-TCO, but full conversion was almost always observed. Peak areas (used for doxorubin quantification) were determined at A=470-500 nm where characteristic doxorubicin absorption takes place and peak integration was done by hand.

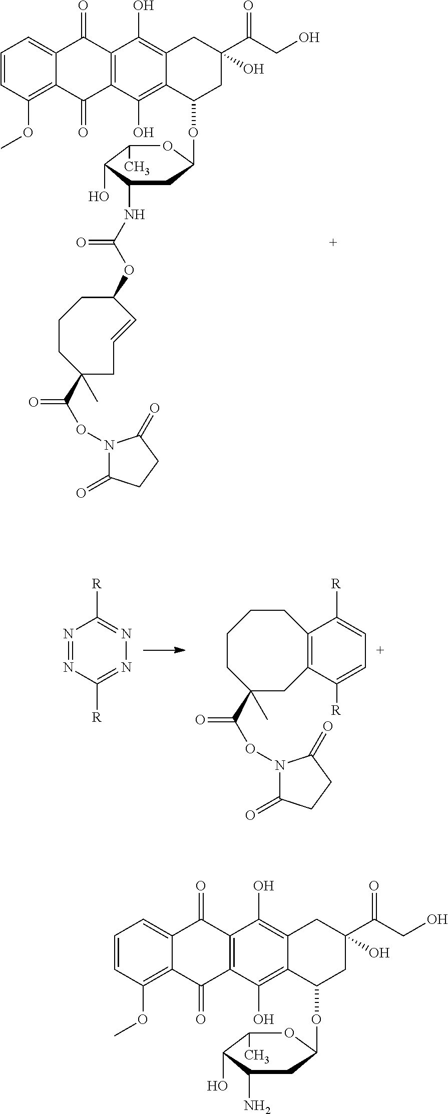

### Example 4

In a similar fashion as Example 3, the release of doxorubicin from TCO-1-doxorubucin as induced by tetrazines 1,8,9 in PBS/ACN and in serum was measured. From Table 1 it is clear that tetrazine 8 affords the highest release and that the release yields are retained when testing in serum.

Serum experiments were conducted as follows:

TCO-1-doxorubucin (6.25×10⁻⁸ mol) was dissolved in DMSO (0.050 mL), and PBS (0.475 mL) was added slowly in aliquots of 0.010 mL, followed by mouse serum (0.475 mL). A portion of this mixture (0.200 mL) was equilibrated at 37° C., and a solution of tetrazine (1.25×10⁻⁷ mol) in DMSO (0.005 mL) was added, and the solution was thoroughly mixed and incubated at 37° C. in the dark for 4 h. Subsequently, cold MeCN (0.200 mL) was added, followed by centrifugation at 13400 rpm for 5 min. The supernatant was used for further analysis by HPLCMS/PDA analysis to determine the release of doxorubicin.

TABLE 1
Doxorubicin release (%) from Axial-TCO-1-Doxorubicin following
addition of 10 equiv. tetrazine 1, 9, 8 in 25% MeCN in PBS or
50% serum at 37° C.; measured with LCMS at 4 h (n = 3).

| | PBS/MeCN | |
|---|---|---|
| Probe | (3/1) | Serum |
| 1 | 7 ± 3 | 12 ± 1 |
| 9 | 55 ± 4 | 46 ± 3 |
| 8 | 79 ± 3 | 75 ± 4 |
| —^{[a]} | 0 | 0 |

^{[a]}no release of doxorubicin from Axial-TCO-1-Doxorubicin at 37° C. in PBS (72 h) and serum (24 h).

### Example 5

### Versatility of the TCO Linker

To demonstrate the versatility of the TCO linker, the stability of a range of TCO derivatives as model compounds was tested under various conditions. In addition, the tetrazine-induced TCO activation was studied under the same conditions. The results in Tables 2-4 support the versatility of the TCO linker and at the same time demonstrate that in addition to aromatic and aliphatic carbamates also carbonates and aromatic and aliphatic esters and ethers are effectively cleaved from the TCO upon tetrazine reaction. In addition to amines, also hydroxy and carboxylic acids form stable conjugates with TCO and can subsequently be cleaved in a range of conditions.

### Typical Example for Testing the Stability of a TCO Compound

The TCO stock solution (10 μL 25 mM; 2.5*10⁻⁷ mol) was added to a solution of the specific condition (100 μL). The mixture was stirred at the specific condition for a certain amount of time, and then the fate of the TCO compound was monitored by HPLC-MS/PDA analysis and/or GC-MS analysis, and an estimation of its stability was made.

### Typical Example for Testing the Feasibility of the Deprotection

The TCO stock solution (10 μL 25 mM in acetonitrile; 2.5*10⁻⁷ mol) was added to a solution of the specific condition (100 μL). A solution of 3,6-dimethyl-1,2,4,5-tetrazine (8, 20 uL 25 mM in acetonitrile; 5.0*10⁻⁷ mol) was added, and the mixture was stirred at the specific condition for a certain amount of time. The reaction was monitored by HPLC-MS/PDA analysis and/or GC-MS analysis, and the percentage of deprotection was estimated.

Conditions: A) in acetonitrile with 5 equivalents of pyridine per TCO at 20° C.

B) in acetonitrile with 5 equivalents of DIPEA per TCO at 20° C.
C) in acetonitrile with 5 equivalents of piperidine per TCO at 20° C.
D) in acetonitrile with 5 equivalents of n-butylamine per TCO at 20° C.
E) in acetonitrile with 5 equivalents of 2-mercaptoethanol per TCO at 20° C. F) in tetrahydrofuran with 5 equivalents of triphenylphosphine per TCO at 20° C. G) in acetonitrile with 5 equivalents of DCC per TCO at 20° C.
H) in acetonitrile with 5 equivalents of PyBOP per TCO at 20° C.
I) in acetonitrile with 1 v % of formic acid at 20° C.
J) in chloroform at 20° C.
K) in chloroform with 1 v % of formic acid at 20° C.
L) in chloroform with 1 v % of trifluoroacetic acid at 20° C.
M) in chloroform with 10 v % of trifluoroacetic acid at 20° C.
N) in chloroform with 33 v % of trifluoroacetic acid at 20° C.
Z) in 25% acetonitrile in water at 20° C.

TABLE 2

| | | Stability of | Deprotection | Deprotected |
|---|---|---|---|---|
| TCO | Condition | TCO | (%) | product |
| 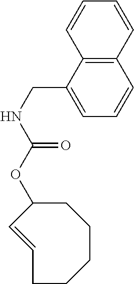 | A) for 1 h B) for 1 h C) for 1 h D) for 1 h E) for 1 h F) for 1 h G) for 1 h H) for 1 h I) for 1 h J) for 1 h K) for 1 h L) for 1 h M) for 1 h Z) for 1 h | stable stable stable stable stable stable stable stable stable stable stable stable stable stable | 87 58 48 47 67 75 74 74 93 73 98 99 99 85 | 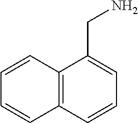 [SMILES: NCc1cccc2ccccc12] |
| axial isomer | | | | |
| 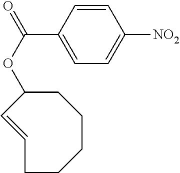 | A) for 1 h B) for 1 h G) for 1 h I) for 1 h Z) for 1 h | stable ca. 2% hydrolysis stable stable stable | 68 83 89 95 80 | 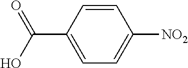 [SMILES: O=C(O)c1ccc([N+](=O)[O-])cc1] |
| axial isomer | | | | |
| 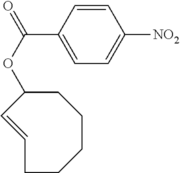 | Z) for 1 h | stable | 66 | 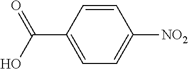 [SMILES: O=C(O)c1ccc([N+](=O)[O-])cc1] |
| equatorial isomer | | | | |
| 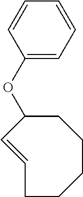 | A) for 1 h B) for 1 h G) for 1 h I) for 1 h Z) for 1 h | stable stable stable stable stable | 42 92 80 92 87 | 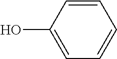 |
| axial isomer | | | | |
| 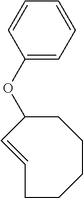 | Z) for 1 h | stable | 72 | 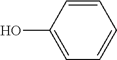 |
| equatorial isomer | | | | |

TABLE 3

| | | Stability of | Deprotection | Deprotected |
|---|---|---|---|---|
| TCO | Condition | TCO | (%) | product |
| 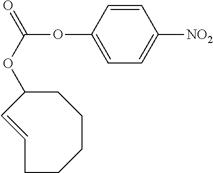 | A) for 1 h B) for 1 h G) for 1 h I) for 1 h Z) for 1 h | stable 19% hydrolysis stable stable stable | 64 90 72 97 70 | 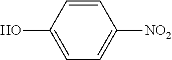 |
| axial isomer | | | | |
| 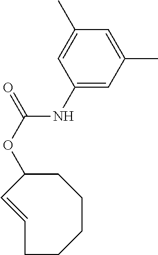 | A) for 1 h B) for 1 h G) for 1 h I) for 1 h | stable stable stable stable | 14 4 61 97 | 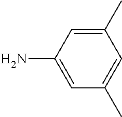 |
| axial isomer | | | | |
| 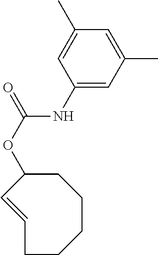 | A) for 1 h B) for 1 h G) for 1 h I) for 1 h | stable stable stable stable | 55 39 91 97 | 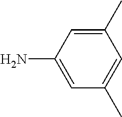 |
| equatorial isomer | | | | |
| 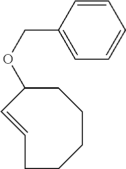 | J) for 1 h Z) for 1 h | stable stable | 70 54 | 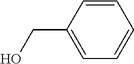 |
| axial isomer | | | | |
| 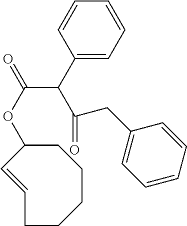 | J) for 1 h Z) for 1 h | stable stable | 95 70 | 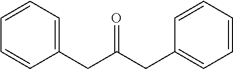 [SMILES: O=C(Cc1ccccc1)Cc1ccccc1] |
| axial isomer | | | | |

TABLE 4

| | | Stability of | Deprotection | Deprotected |
|---|---|---|---|---|
| TCO | Condition* | TCO | (%) | product |
| 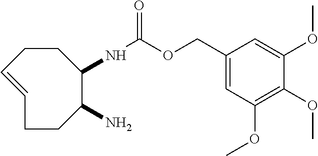 | 10% MeCN in H₂O for 5 day 0.1M NH₄OAc buffer, pH = 7.0 for 5 day 0.1M HCOOH buffer, pH = 2.0 for 2 day 0.1M NH₃ buffer, pH = 11 for 5 day | stable stable stable <5% cyclization | 0% 0% 0% 87% | 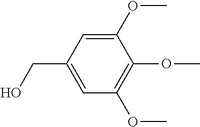 |
| | 10% MeCN in H₂O | <5% | 50% | |
| | at 70° C. for 5 day | cyclization | | |
| | 0.1M NH₄OAc | stable | 0% | |
| | buffer, pH = 8.0 for | | | |
| | 8 day | | | |
| | 0.1M NH₄OAc | stable | 25% | |
| | buffer, pH = 9.0 for | | | |
| | 8 day | | | |
| | 0.1M NH₄OAc | stable | 81% | |
| | buffer, pH = 10.0 for | | | |
| | 8 day | | | |
| 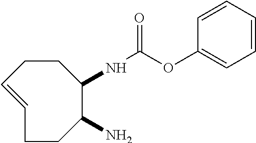 | 0.1M NH₄OAc buffer, pH = 7.0 0.1M NH₄OAc buffer, pH = 8.0 0.1M NH₄OAc buffer, pH = 9.0 with 5 eq. of pyridine in MeCN for 16 h with 5 eq. of DIPEA | t_{1/2} = 80 h t_{1/2} = 17 h t_{1/2} = 5.3 h 2.6% cyclization 6.7% | t_{1/2} = 140 min t_{1/2} = 25 min t_{1/2} = 4.6 min 71.7% 98.8% | 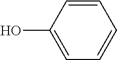 |
| | in MeCN for 16 h | cyclization | | |
| | with 5 eq. of PyBOP | 5.9% | 24.4% | |
| | in MeCN for 16 h | cyclization | | |
| | with 5 eq. of | 1.9% | 33.9% | |
| | HCOOH in MeCN | cyclization | | |
| | for 16 h | | | |
| 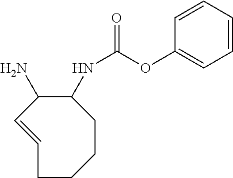 | in PBS buffer | t_{1/2} = 65 h | t_{1/2} = 70 min | 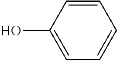 |
| 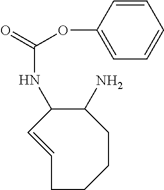 | in PBS buffer | t_{1/2} = 29 h | t_{1/2} = 100 min | 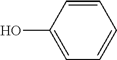 |

*study is performed at 20° C., unless stated otherwise

### Example 6

### Activation of Tumor-Bound T-Cell Engaging Triabody

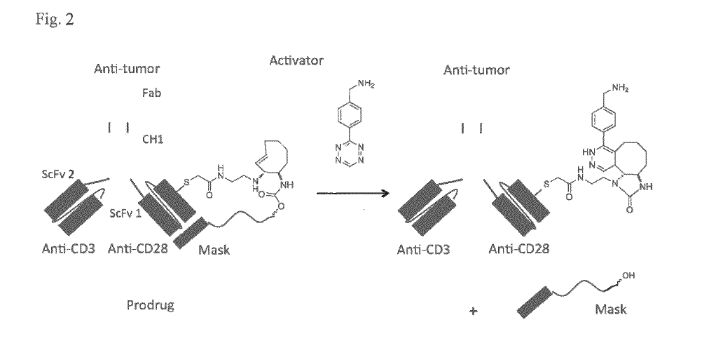

The triabody comprises a tumor-binding moiety, a CD3 T-cell engaging moiety, and a CD28 T-cell co-stimulatory moiety. As the CD3 and CD28 combined in one molecule will result in unacceptable toxic effect off target, the anti-CD28 domain is blocked by a Masking Moiety M^{M}, a peptide resembling the CD28 binding domain and which has affinity for the anti-CD28 moiety. This peptide is linked through a further peptide or a PEG chain L^{D} and/or S^{P} to the TCO trigger which is itself conjugated to a site specifically engineered cysteine. After Prodrug administration, tumor binding and clearance from blood, the Activator is injected. The reaction of the Activator with the TCO trigger in the Prodrug results in release of the Masking Moiety from the anti-CD28 domain enabling CD28 co-stimulation of T-cells, boosting the T-cell mediated anticancer effect, while avoiding off target toxicity. A schematic drawing showing the prodrug, comprising the mask and the Trigger, and the result of the reaction with the Activator, which leads to the release of the Mask, is shown in FIG. 2.

### Example 7

The person skilled in the art is aware of the available methods to design a suitably antibody-linked M^{M} such that the M^{M} has a non-covalent intramolecular interaction or binding with the CDR of the antibody. The correct affinity between M^{M} and CDR and how to determine the length of M^{M}-(L^{D}) and therefore the antibody-conjugation position has been described in amongst other WO2013163631, US20100189651, US20100221212, and Thomas et al., Protein Science 2009, 18:2053-2059. In addition there are several methods to achieve site selective antibody amino acid engineering, see for example Axup et al., PNAS 2012, 109, 16101-16106 and Junutula et al., Nature Biotechnology, 26, 925. Site selective introduction of artificial aminoacids (containing e.g. an aldehyde, alkyne or azide) allow site selective M^{M}-(L^{D}) antibody conjugation. Above mentioned sources also provide locations and teach how to select aminoacid positions near the CDR of the antibody that can be modified without adversely affecting antibody function, such as CDR binding to its target.

What is claimed is:

1. A kit for the administration and activation of a Prodrug, the kit comprising a Masking Moiety linked, directly or indirectly, to a Trigger moiety, which in turn is linked to a Drug, and an Activator for the Trigger moiety, wherein the Trigger moiety comprises a dienophile and the Activator comprises a diene, the dienophile satisfying the following formula (1a): 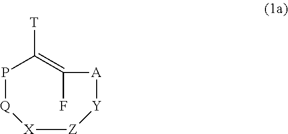
wherein T, F each independently denotes H, or a substituent selected from the group consisting of alkyl, F, Cl, Br or I;
A and P each independently are CR^{a}₂ or CR^{a}X^{D}, provided that at least one is CR^{a}X^{D}; X^{D} is (O—C(O))ₚ-(L^{D})ₙ-(D^{D}), S—C(O)-(L^{D})ₙ-(D^{D}), O—C(S)-(L^{D})ₙ-(D^{D}), S—C(S)-(L^{D})ₙ-(D^{D}), or O—S(O)-(L^{D})ₙ-(D^{D}), wherein p=0 or 1; (L^{D})ₙ is an optional linker, with n=0 or 1, preferably linked to T^{R} via S, N, NH, or O, wherein these atoms are part of the linker, which may consist of multiple units arranged linearly and/or branched;
Y, Z, Q, X together form a four-membered aliphatic or heteroaliphatic moiety, optionally fused to an aromatic moiety or moieties;
each R^{a} independently is selected from the group consisting of H, alkyl, aryl, OR′, SR′, S(═O)R′″, S(═O)₂R′″, S(═O)₂NR′R″, Si—R″, Si—O—R′″, OC(═O)R′″, SC(═O)R′″, OC(═S)R′″, SC(═S)R′″, F, Cl, Br, I, N₃, SO₂H, SO₃H, SO₄H, PO₃H, PO₄H, NO, NO₂, CN, OCN, SCN, NCO, NCS, CF₃, CF₂—R′, NR′R″, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R″, NR′C(═O)S—R″, NR′C(═S)S—R′″, OC(═O)NR′—R′″, SC(═O)NR′—R′″, OC(═S)NR′—R′″, SC(═S)NR′—R′″, NR′C(═O)NR″—R″, NR′C(═S)NR″—R″, CR′NR″, with each R′ and each R″ independently being H, aryl or alkyl and R′″ independently being aryl or alkyl;
each R^{b} is independently selected from the group consisting of H, alkyl, aryl, O-aryl, O-alkyl, OH, C(═O)NR′R″ with R′ and R″ each independently being H, aryl or alkyl, R′CO-alkyl with R′ being H, alkyl, and aryl;
each R^{c} is independently selected from the group consisting of H, alkyl, aryl, O-alkyl, D-aryl, OH;
wherein two or more R^{a,b,c} moieties together may form a ring;
D^{D} is one or more therapeutic moieties or drugs, preferably linked via S, N, NH, or O, wherein these atoms are part of the therapeutic moiety.

2. A kit according to claim 1, wherein the dienophile satisfies the following formula (1a): 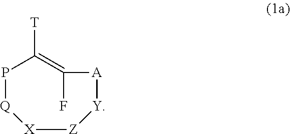
wherein A and P each independently are CR^{a}₂ or CR^{a}X^{D}, provided that at least one, and preferably not more than one, is CR^{a}X^{D}. X^{D} is (O—C(O))ₚ-(L^{D})ₙ-(M^{M}),)S—C(O)-(L^{D})ₙ-(M^{M}), O—C(S)-(L^{D})ₙ-(M^{M}), S—C(S)-(L^{D})ₙ-(M^{M}), O—S(O)-(L^{D})ₙ-(M^{M}), wherein p=0 or 1. Preferably, X^{D} is (O—C(O))ₚ-(L^{D})ₙ-(M^{M}), where p=0 or 1, preferably 1, and n=0 or 1; Y, Z, X, Q each independently are selected from the group consisting of CR^{a}₂, C═CR^{a}₂, C═O, C═S, C═NR^{b}, S, SO, SO₂, O, NR^{b}, and SiR^{c}₂, with at most three of Y, Z, X, and Q being selected from the group consisting of C═CR^{a}₂, C═O, C═S, and C═NR^{b}, wherein two R moieties together may form a ring, and with the proviso that no adjacent pairs of atoms are present selected from the group consisting of O—O, O—NR^{b}, S—NR^{b}, O—S, O—S(O), O—S(O)₂, and S—S, and such that Si is only adjacent to CR^{a}₂ or O.

3. A kit according to claim 1, wherein the dienophile satisfies the following formula (1a): 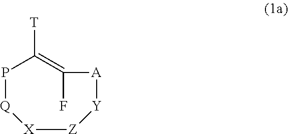
wherein A and P each independently are CR^{a}₂ or CR^{a}X^{D}, provided that at least one, and preferably not more than one, is CR^{a}X^{D}. X^{D} is (O—C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), O—S(O)-(L^{D})ₙ-(M^{M}), where in p=0 or 1. Preferably, X^{D} is (O—C(O))ₚ-(L^{D})ₙ-(M^{M}), where p=0 or 1, preferably 1, and n=0 or 1; wherein one of the bonds PQ, QX, XZ, ZY, YA is part of a fused ring or consists of CR^{a}═CR^{a}, such that two exocyclic bonds are fixed in the same plane, and provided that PQ and YA are not part of an aromatic 5- or 6-membered ring, of a conjugated 7-membered ring, or of CR^{a}═CR^{a};
when not part of a fused ring P and A are independently CR^{a}₂ or CR^{a}X^{D}, provided that at least one, and preferably not more than one, is CR^{a}X^{D}; when part of a fused ring P and A are independently CR^{a} or CX^{D}, provided that at least one, and preferably not more than one, is CX^{D}; the remaining groups (Y, Z, X, Q) being independently from each other CR^{a}₂, C═CR^{a}₂, C═O, C═S, C═NR^{b}, S, SO, SO₂, O, NR^{b}, SiR^{c}₂, such that at most 1 group is C═CR^{a}₂, C═O, C═S, C═NR^{b}, and no adjacent pairs of atoms are present selected from the group consisting of O—O, O—NR^{b}, S—NR^{b}, O—S, O—S(O), O—S(O)₂, and S—S, and such that Si, if present, is adjacent to CR^{a}₂ or O, and the CR^{a}₂═CR^{a}₂ bond, if present, is adjacent to CR^{a}₂ or C═CR^{a}₂ groups.

4. A kit according to any one of the preceding claims, wherein the dienophile is a trans-cyclooctene moiety that satisfies formula (1 b): 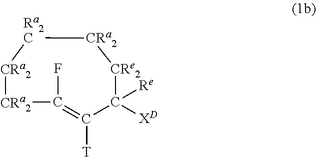
wherein, in addition to the optional presence of at most two exocyclic bonds fixed in the same plane, each R^{a} independently denotes H, or, in at most four instances, a substituent selected from the group consisting of alkyl, aryl, OR′, SR′, S(═O)R′″, S(═O)₂R′″, S(═O)₂NR′R″, Si—R″, Si—O—R′″, OC(═O)R′″, SC(═O)R′″, OC(═S)R′″, SC(═S)R′″, F, Cl, Br, I, N₃, SO₂H, SO₃H, SO₄H, PO₃H, POOH, NO, NO₂, CN, OCN, SCN, NCO, NCS, CF₃, CF₂—R′, NR′R″, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R′″, NR′C(═O)S—R′″, NR′C(═S)S—R′″, OC(═O)NR′—R′″, SC(═O)NR′—R′″, OC(═S)NR′—R′″, SC(═S)NR′—R′″, NR′C(═O)NR″—R″, NR′C(═S)NR″—R″, CR′NR″, with each R′ and each R″ independently being H, aryl or alkyl and R′″ independently being aryl or alkyl;
wherein each Re as above indicated is independently selected from the group consisting of H, alkyl, aryl, OR′, SR′, S(═O)R′″, S(═O)₂R′″, Si—R″, Si—O—R′″, OC(═O)R′″, SC(═O)R′″, OC(═S)R′″, SC(═S)R′″, F, Cl, Br, I, N₃, SO₂H, SO₃H, PO₃H, NO, NO₂, CN, CF₃, CF₂—R′, C(═O)R′, C(═S)R′, C(═O)O—R′, C(═S)O—R′, C(═O)S—R′, C(═S)S—R′, C(═O)NR′R″, C(═S)NR′R″, NR′C(═O)—R′″, NR′C(═S)—R′″, NR′C(═O)O—R′″, NR′C(═S)O—R′″, NR′C(═O)S—R′″, NR′C(═S)S—R′″, NR′C(═O)NR″—R″, NR′C(═S)NR″—R″, CR′NR″, with each R′ and each R″ independently being H, aryl or alkyl and R′″ independently being aryl or alkyl;
wherein two R^{a,e} moieties together may form a ring; wherein one R^{a,e} or the self-immolative linker L^{D}, is bound, optionally via a spacer or spacers S^{P}, to the species D^{D}, and wherein T and F each independently denote H, or a substituent selected from the group consisting of alkyl, F, Cl, Br, and I, and X^{D} is (O—C(O))ₚ-(L^{D})ₙ-(M^{M}), S—C(O)-(L^{D})ₙ-(M^{M}), O—C(S)-(L^{D})ₙ-(M^{M}), S—C(S)-(L^{D})ₙ-(M^{M}), O—S(O)-(L^{D})ₙ-(M^{M}), where in p=0; or 1. Preferably, X^{D} is (0-C(O))ₚ-(L^{D})ₙ-(M^{M}), where p=0 or 1, preferably 1, and n=0 or 1.

5. A kit according to any one of the preceding claims, wherein the dienophile satisfies any one of the following formulae:

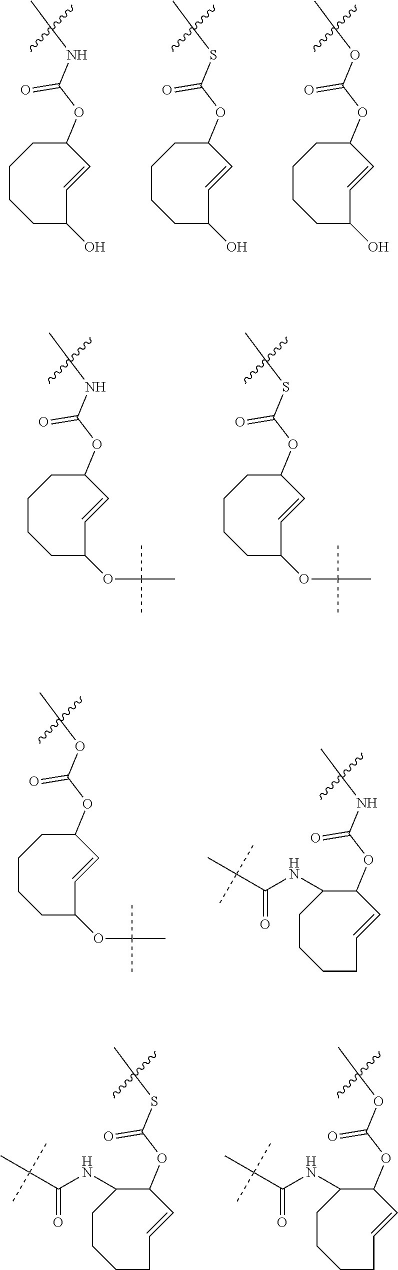

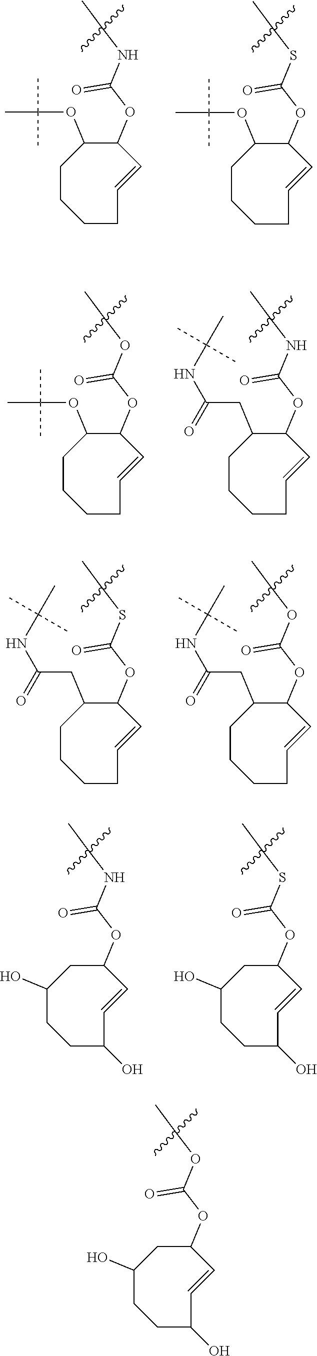

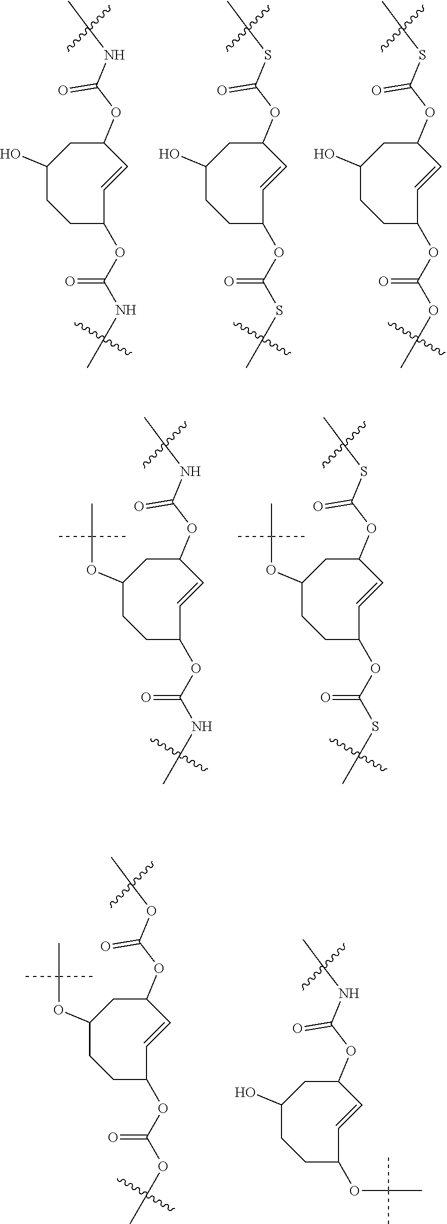

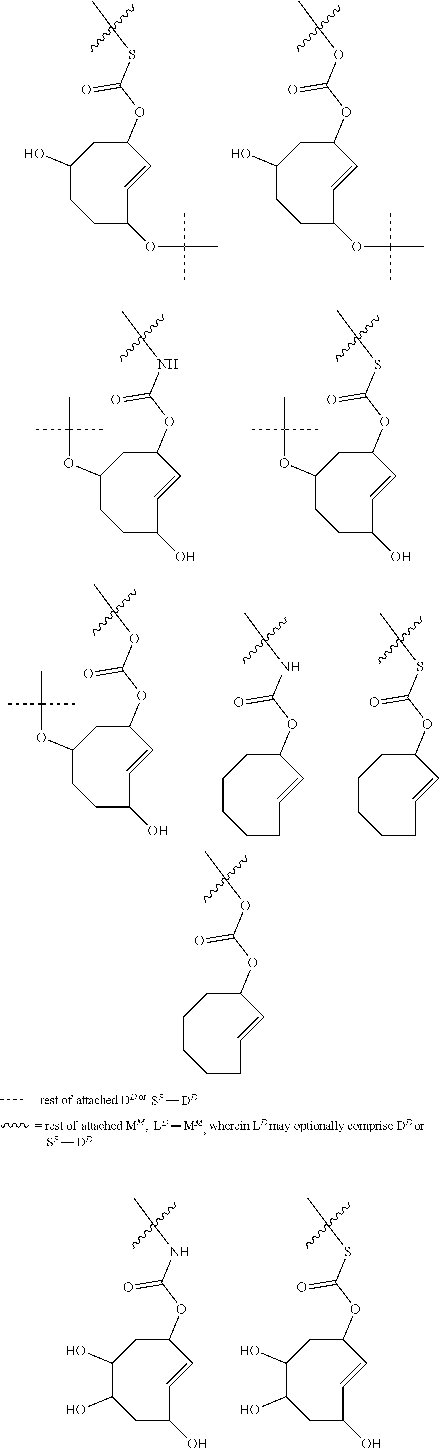

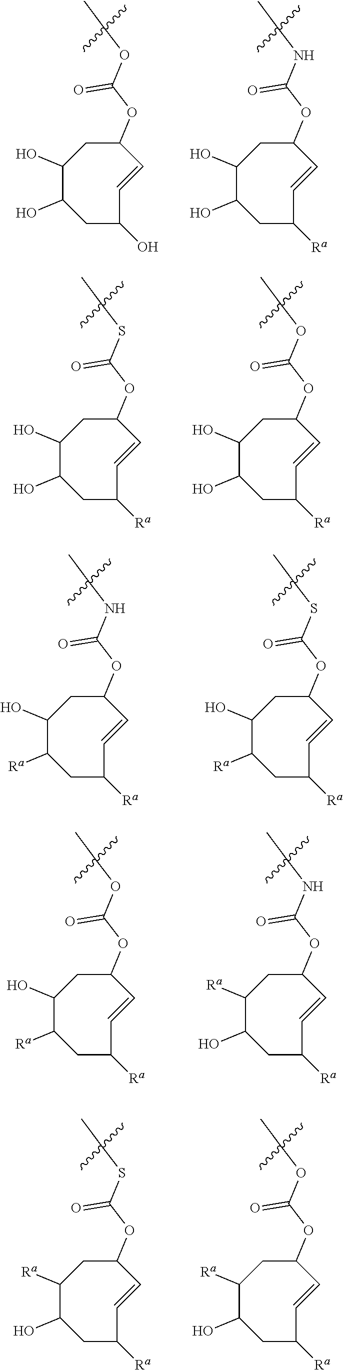

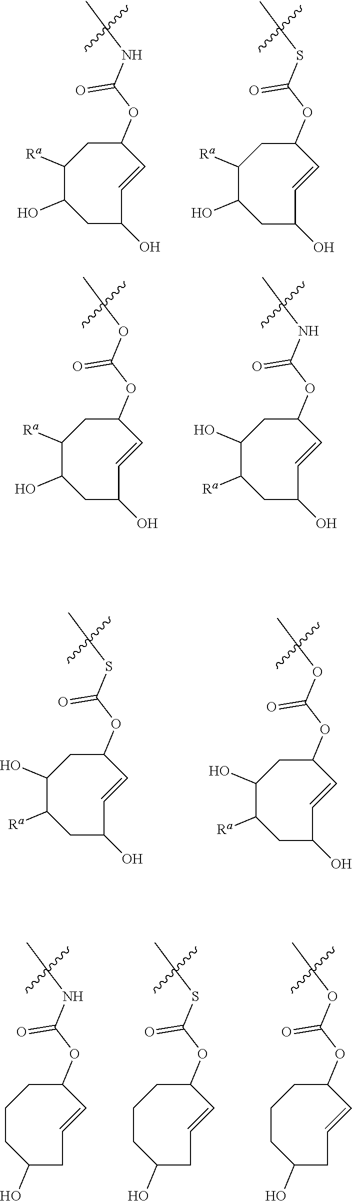

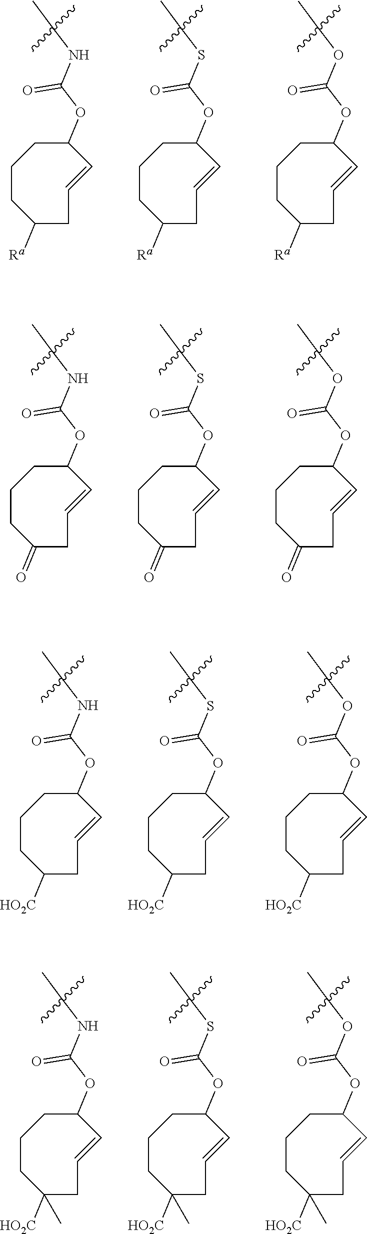

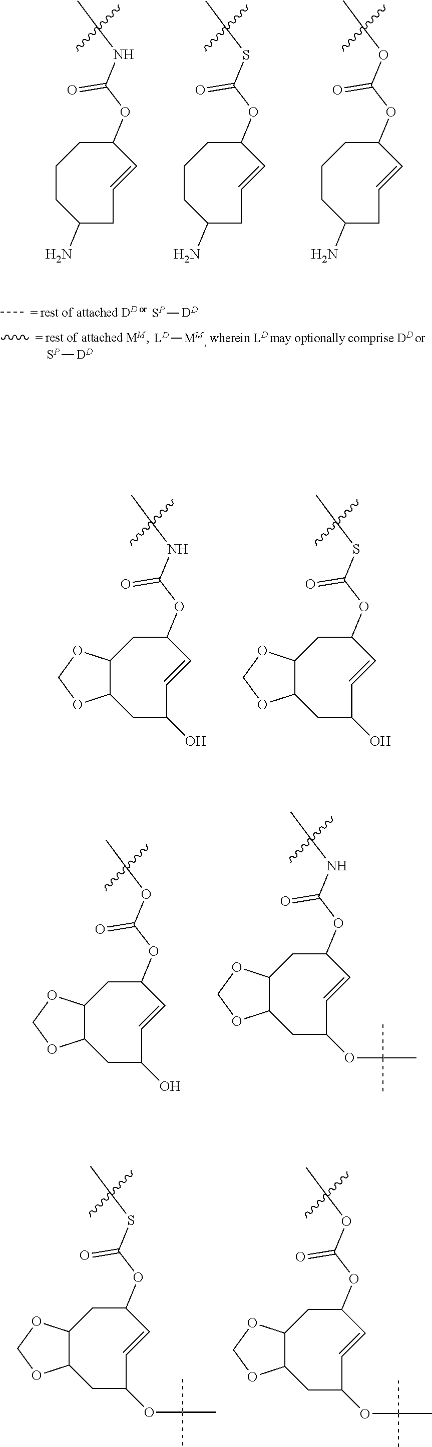

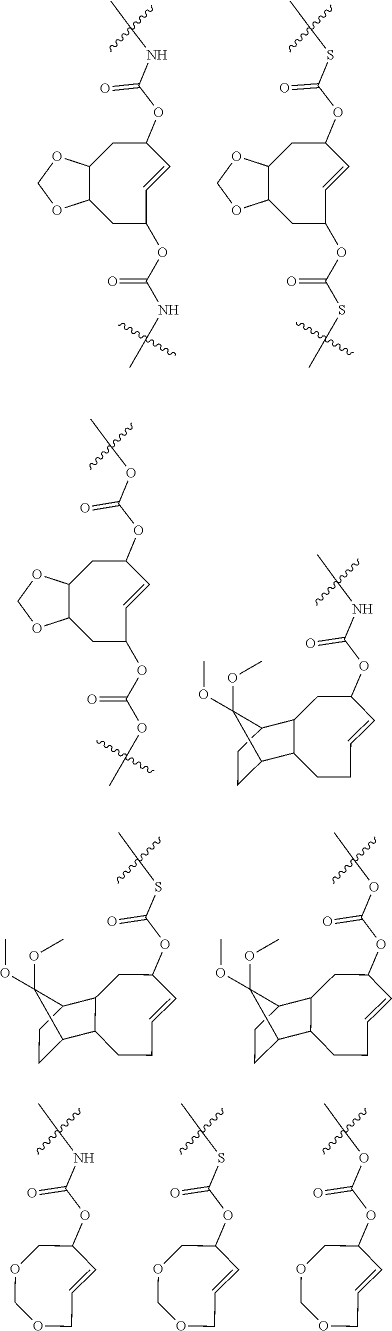

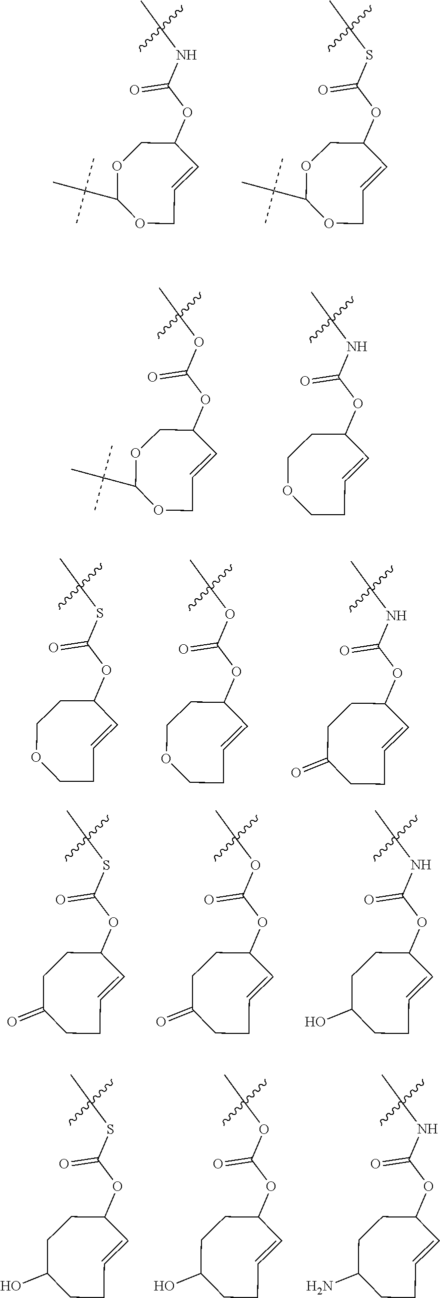

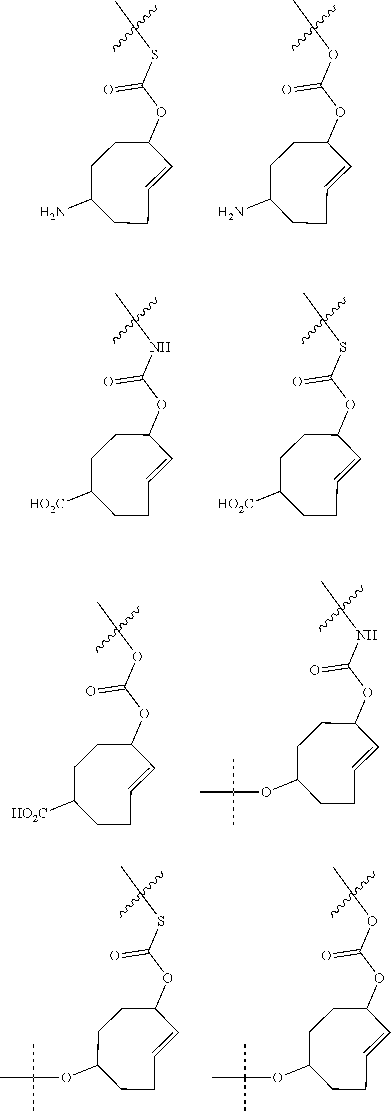

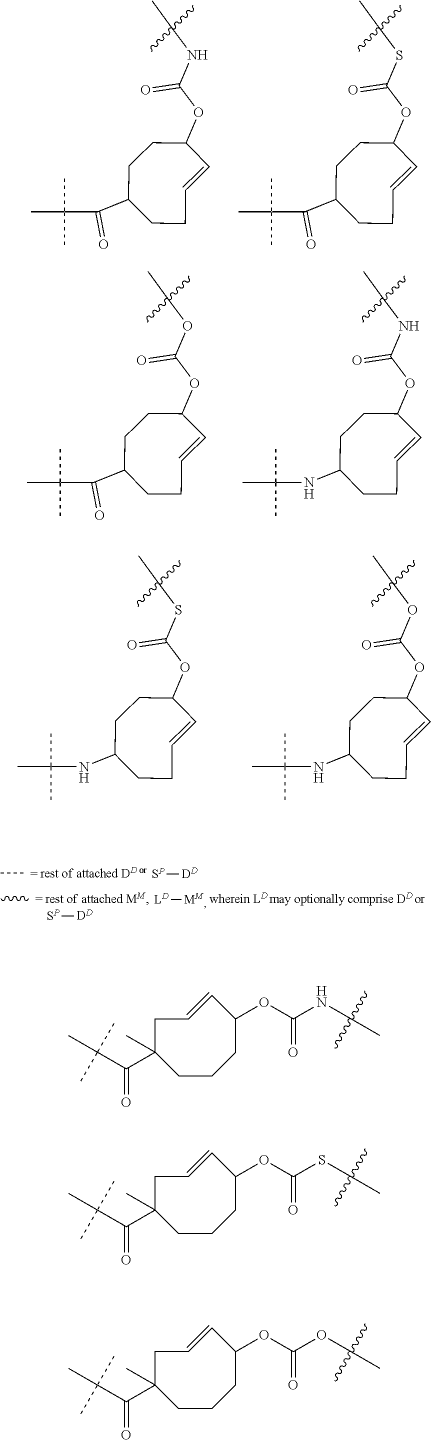

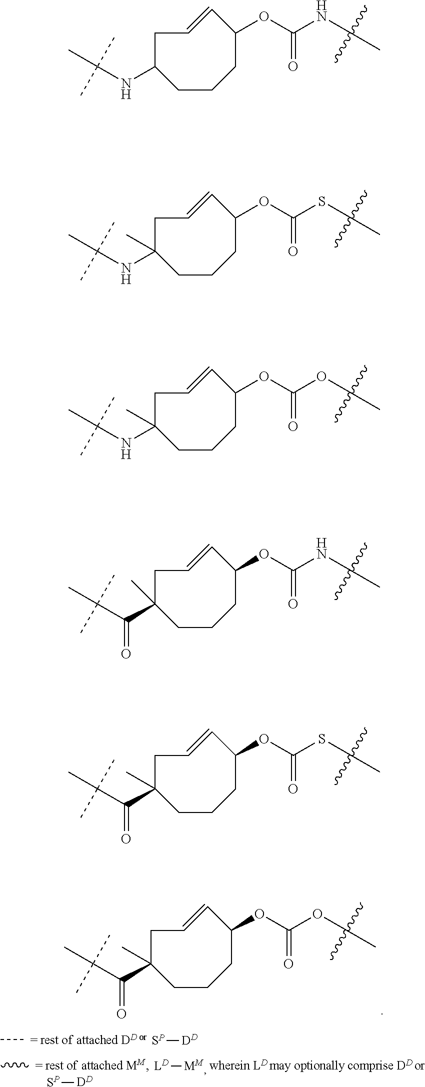

6. A kit according to any one of the claims 1-4, wherein the dienophile satisfies any one of the following formulae:

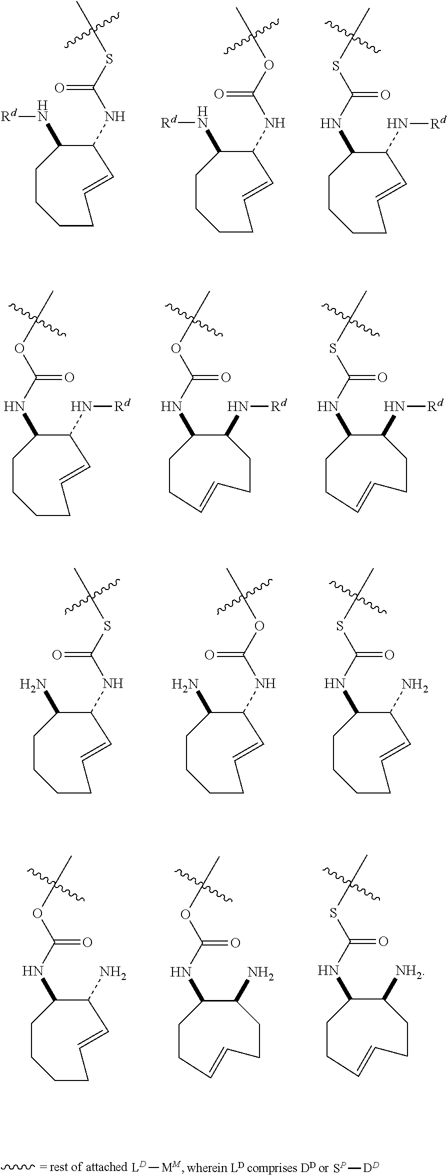

7. A kit according to any one of the claims 1-4, wherein the dienophile satisfies any one of the following formulae:

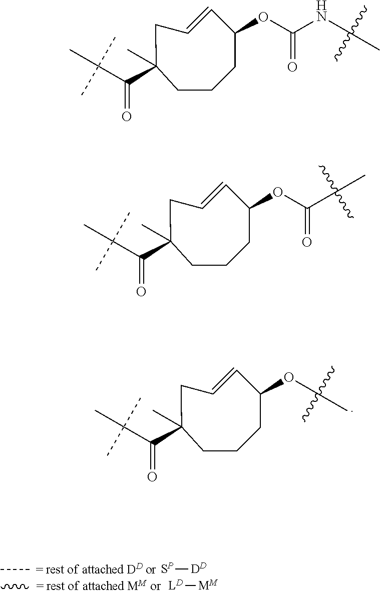

8. A kit according to any one of the claims 1-4, wherein the dienophile comprises the structure:

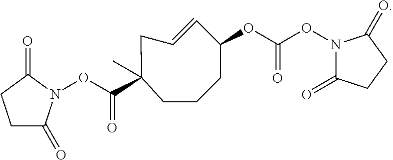

9. A kit according to any one of the claims 1-4, wherein the dienophile comprises either of the following structures:

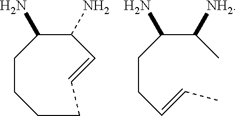

10. A kit according to any one of the preceding claims, wherein the Activator comprises a diene satisfying any one of the following formulae (2) to (4): 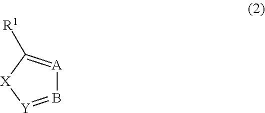
wherein R¹ is selected from the group consisting of H, alkyl, aryl, CF₃, CF₂—R′, OR′, SR′, C(═O)R′, C(═S)R′, C(═O)O—R′, C(═O)S—R′, C(═S)O—R′, C(═S)S—R″, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, NR′C(═O)NR″R″, and NR′C(═S)NR″R″ with each R′ and each R″ independently being H, aryl or alkyl; A and B each independently are selected from the group consisting of alkyl-substituted carbon, aryl substituted carbon, nitrogen, N⁺O⁻, N⁺R with R being alkyl, with the proviso that A and B are not both carbon; X is selected from the group consisting of O, N-alkyl, and C═O, and Y is CR with R being selected from the group consisting of H, alkyl, aryl, C(═O)OR′, C(═O)SR′, C(═S)OR′, C(═S)SR′, C(═O)NR′R″ with R′ and R″ each independently being H, aryl or alkyl; 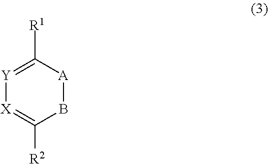
wherein R¹ and R² each independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, and NR′C(═S)NR″R″ with each R′ and each R″ independently being H, aryl or alkyl, and R′″ independently being aryl or alkyl; A is selected from the group consisting of N-alkyl, N-aryl, C═O, and CN-alkyl; B is O or S; X is selected from the group consisting of N, CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, and CC(═S)NR′R″, R′ and R″ each independently being H, aryl or alkyl and R′″ independently being aryl or alkyl; Y is selected from the group consisting of CH, C-alkyl, C-aryl, N, and N⁺O⁻. 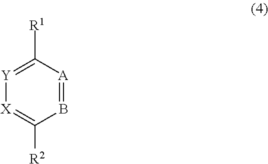
wherein R¹ and R² each independently are selected from the group consisting of H, alkyl, aryl, CF₃, CF₂—R′, NO, NO₂, OR′, SR′, CN, C(═O)R′, C(═S)R′, OC(═O)R′″, SC(═O)R′″, OC(═S)R′″, SC(═S)R′″, S(═O)R′, S(═O)₂R′″, S(═O)₂OR′, PO₃R′R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, and NR′C(═S)NR″R″ with each R′ and each R″ independently being H, aryl or alkyl, and R′″ independently being aryl or alkyl; A is selected from the group consisting of N, C-alkyl, C-aryl, and N⁺O⁻; B is N;
X is selected from the group consisting of N, CH, C-alkyl, C-aryl, CC(═O)R′, CC(═S)R′, CS(═O)R′, CS(═O)₂R′″, CC(═O)O—R′, CC(═O)S—R′, CC(═S)O—R′, CC(═S)S—R′, CC(═O)NR′R″, CC(═S)NR′R″, R′ and R″ each independently being H, aryl or alkyl and R′″ independently being aryl or alkyl; Y is selected from the group consisting of CH, C-alkyl, C-aryl, N, and N⁺O⁻.

11. A kit according to claim 10, wherein the diene satisfies formula (7) as defined in the description.

12. A kit according to claim 10, wherein the diene satisfies formula (8a) or (8b): 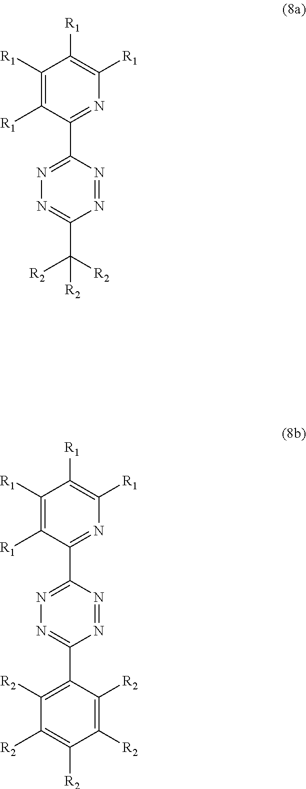
wherein each R¹ and each R² independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, and NR′C(═S)NR″R″ with each R′ and each R″ independently being H, aryl or alkyl, and R′″ independently being aryl or alkyl.

13. A kit according to claim 10, wherein the diene is satisfies a formula selected from the group consisting of (8c), (8d), (8e), (8f), and (8g): 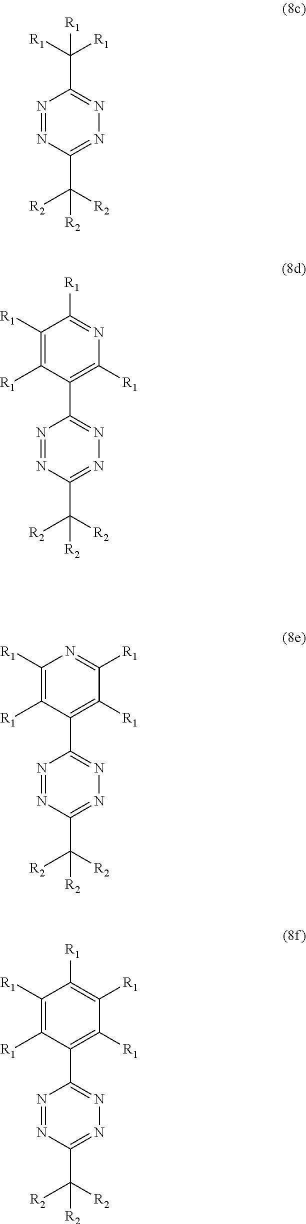 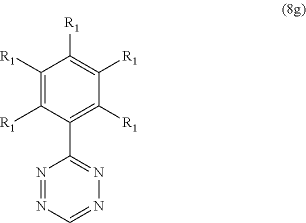
wherein each R¹ and each R² independently are selected from the group consisting of H, alkyl, aryl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)NR′R″, SC(═S)NR′R″, NR′C(═O)NR″R″, and NR′C(═S)NR″R″ with each R′ and each R″ independently being H, aryl or alkyl, and R′″ independently being aryl or alkyl.

14. A kit according to claim 10, wherein the diene satisfies any one of the formulae:

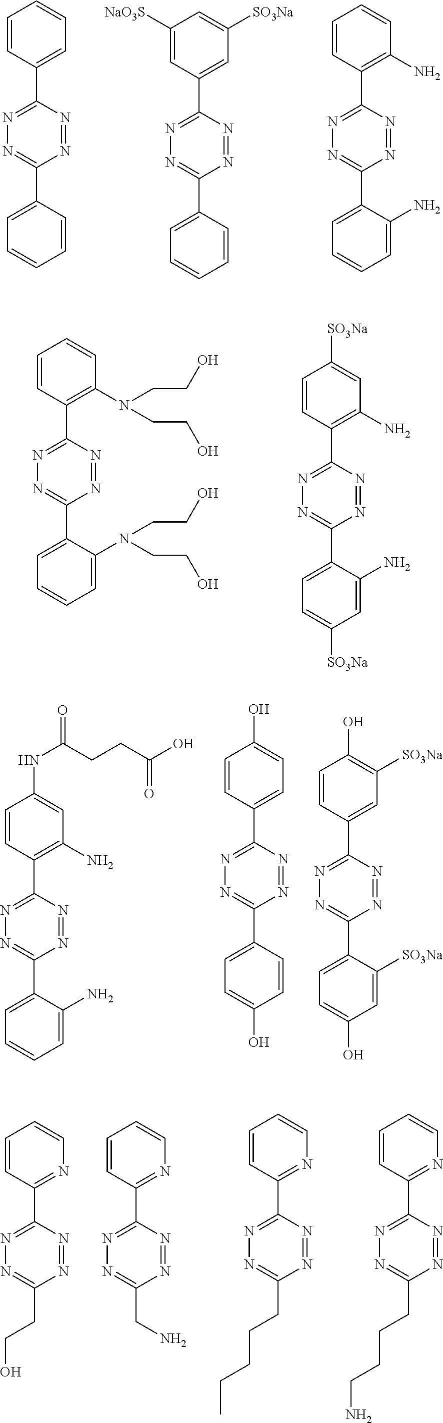

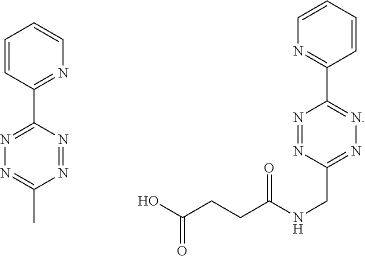 [SMILES: Cc1nnc(-c2ccccn2)nn1 ; O=C(O)CCC(=O)NCc1nnc(-c2ccccn2)nn1]

15. A kit according to claim 10, wherein the diene satisfies any one of the formulae:

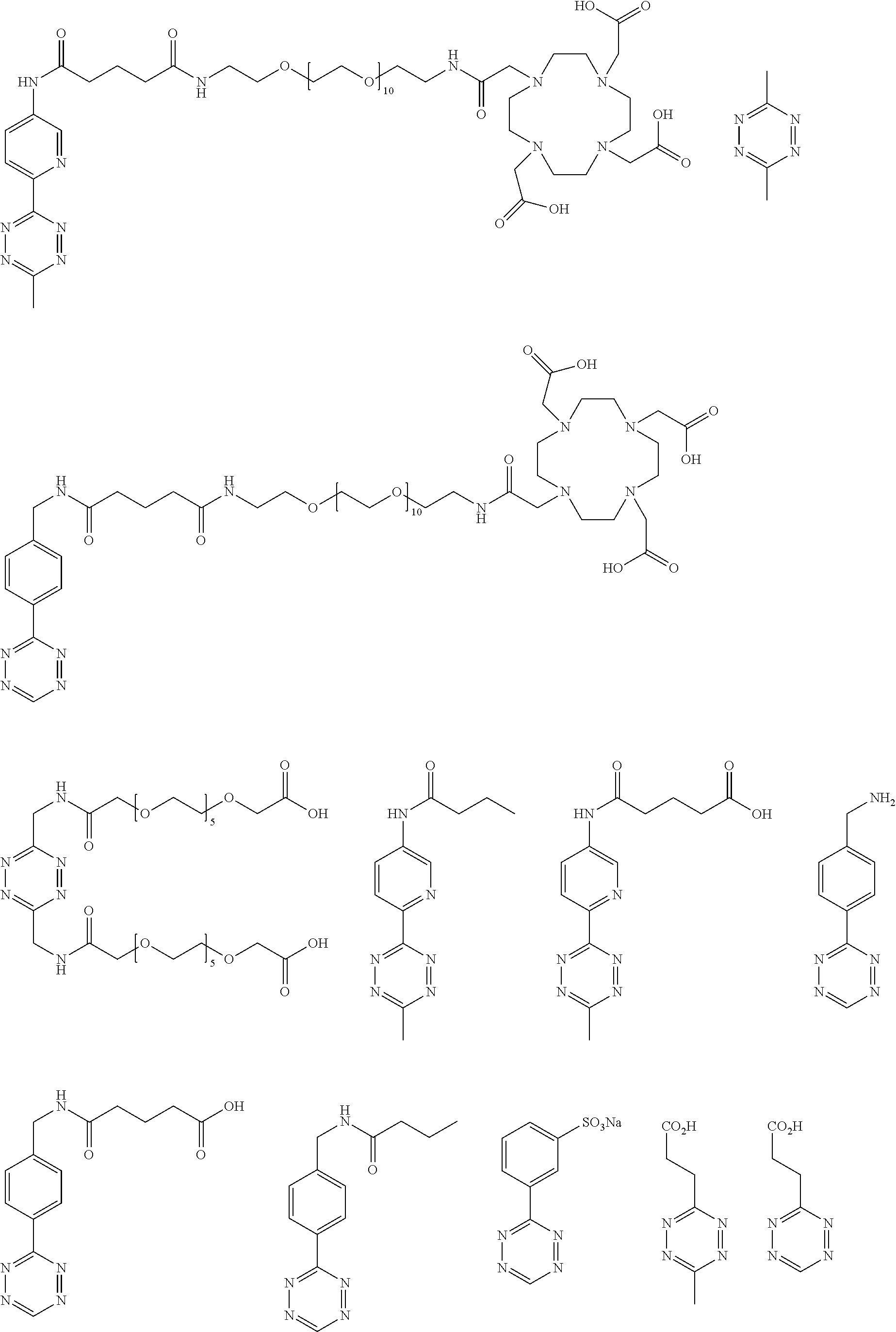

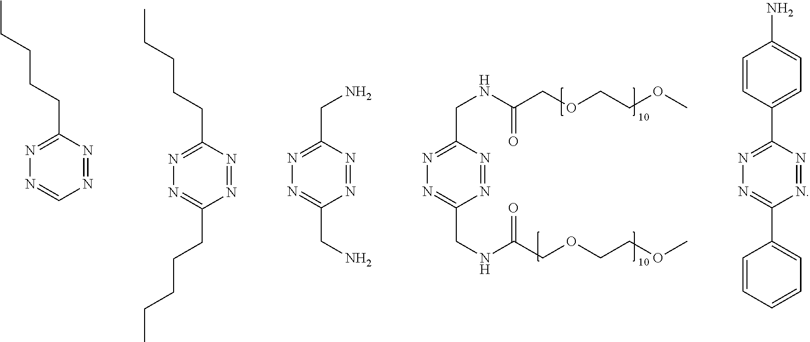

16. A kit according to claim 10, wherein the diene satisfies any one of the formulae:

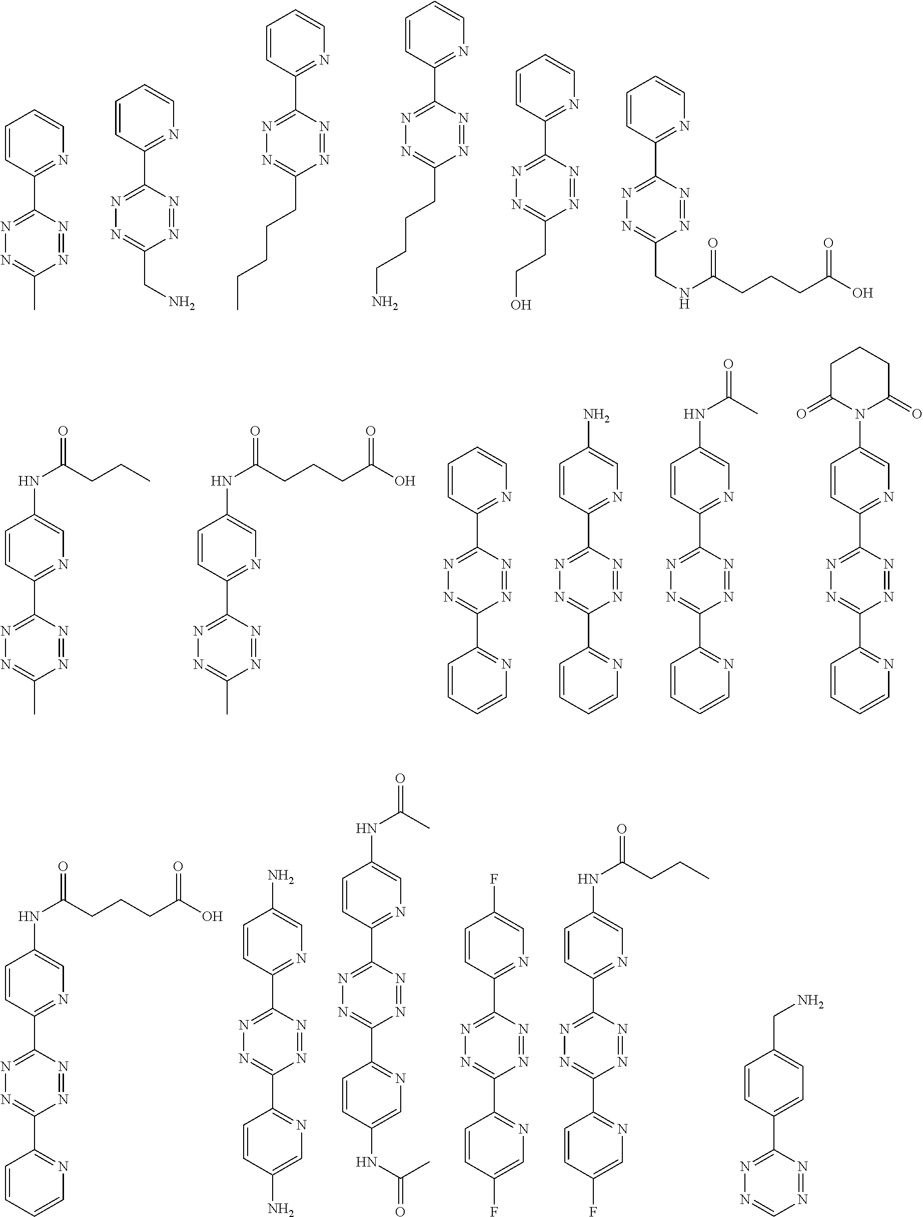

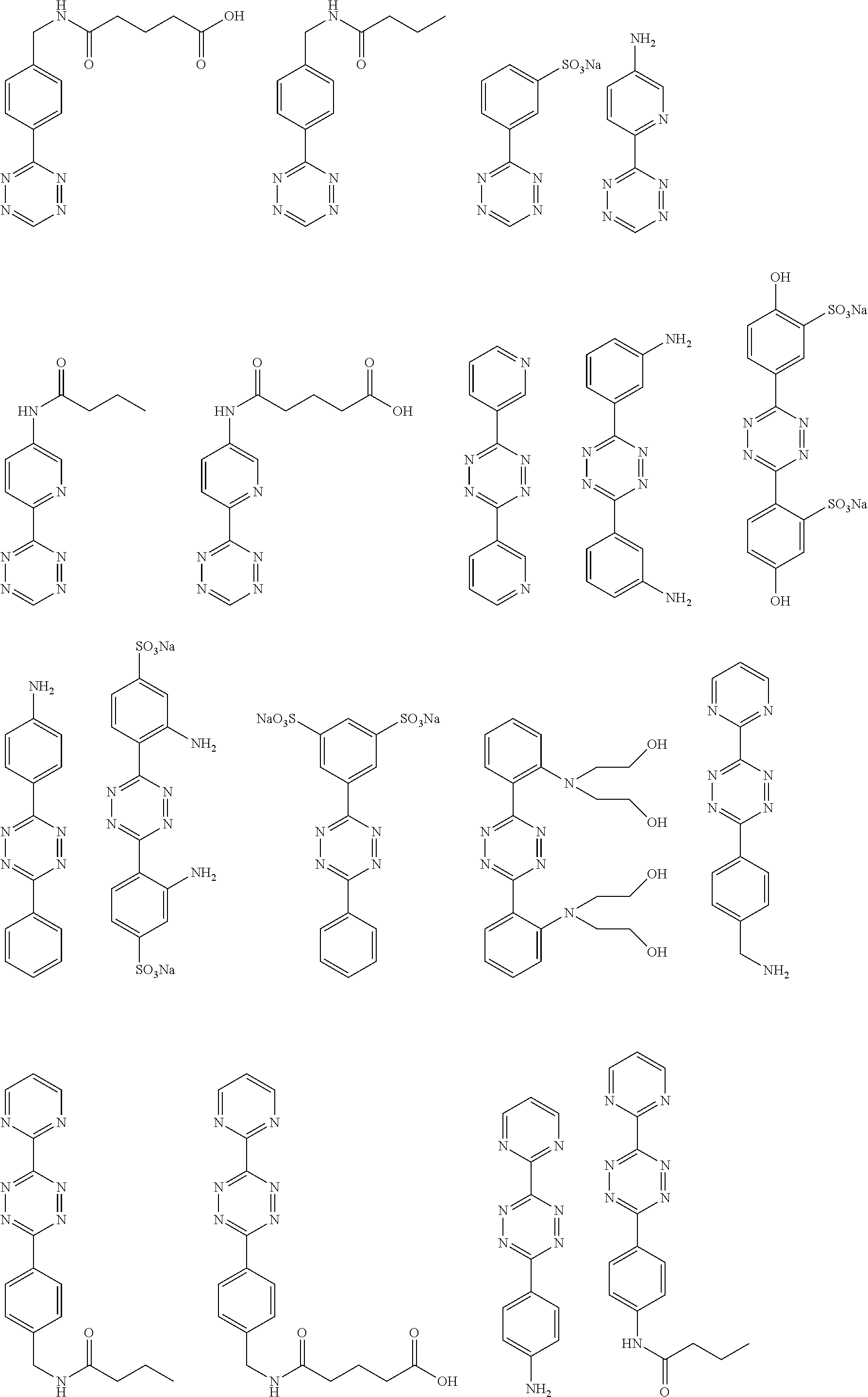

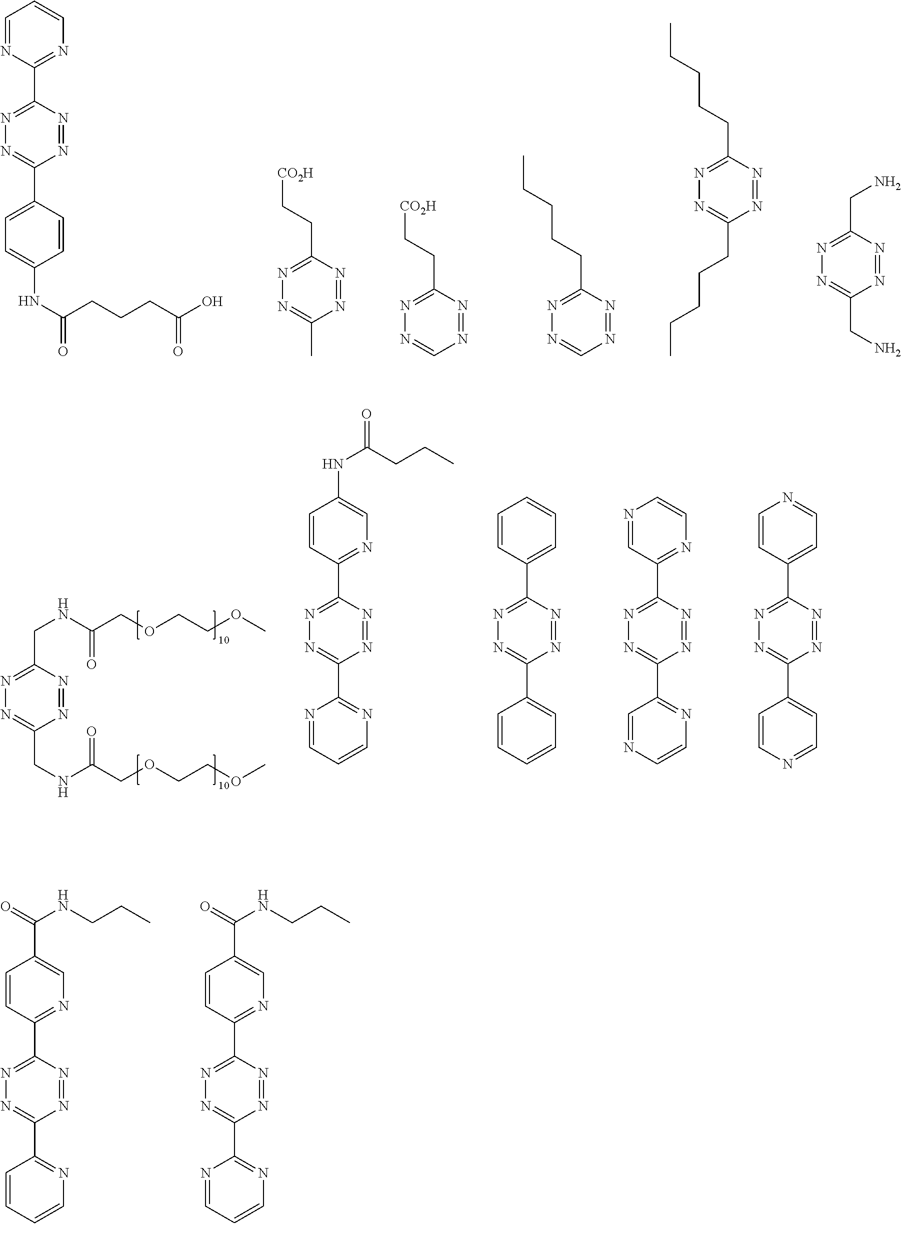

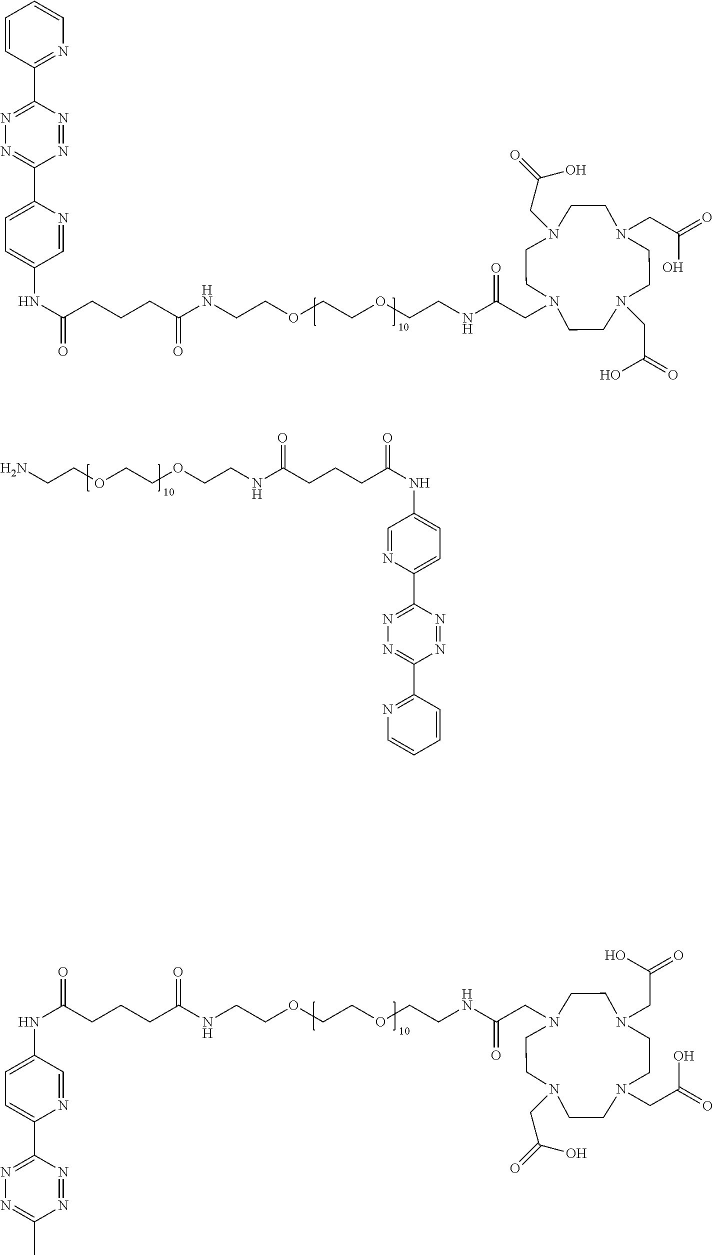

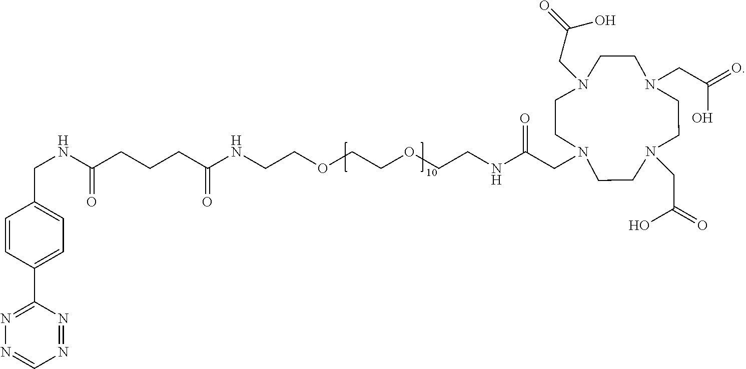

17. A kit according to claim 10, wherein the diene satisfies the formula:

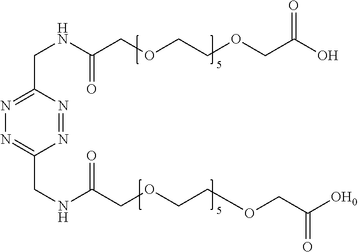

18. A kit according to claim 10, wherein the diene satisfies the formula:

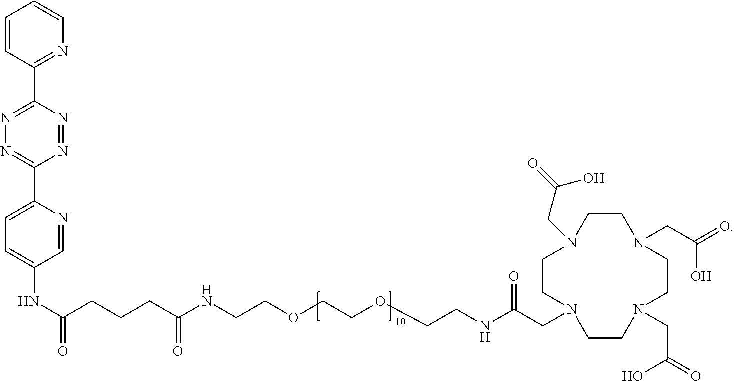

19. A kit according to any one of the preceding claims, wherein the drug is a T-cell engaging antibody construct.

20. A kit according to any one of the preceding claims, wherein the masking moiety is a peptide or a protein.

21. A kit according to any one of the preceding claims, wherein the drug is selected from the group consisting of antibodies, antibody derivatives, antibody fragments, bi-specific mAb fragments and trispecific mAb fragments.
